(19) United States
(12) Patent Application Publication
(10) Pub. No.: US 2015/0374738 A1
(43) Pub. Date: Dec. 31, 2015

(54) COMPOSITIONS AND METHODS FOR REGULATING CELL GROWTH AND DEVELOPMENT

(71) Applicant: AOVART GmbH, Schwanden (CH)

(72) Inventors: Nina M. Gevorkyan, Moscow (RU); Anna G. Babaeva, Moscow (RU); Natalya V. Tishevskaya, Chelyabinsk (RU)

(21) Appl. No.: 14/700,123

(22) Filed: Apr. 29, 2015

(30) Foreign Application Priority Data
Jun. 26, 2014 (RU) 2014126089

(51) Int. Cl. A61K 31/713

(52) U.S. Cl. CPC A61K 31/713

(57) ABSTRACT

The invention relates to the field of basic biology, practical regenerative medicine, veterinary, cell biology and can be used to treat and prevent diseases, disorders or conditions associated with the violation of proliferation and differentiation of cells of different organs and tissues to activate the regeneration potential of human and animal organs and tissues at age-related changes and after extreme impacts, as well as for biomedical research. The present invention can be widely applied in the field of blood transfusion, organ transplantation, as well as serve as a general approach to the development of reliable methods to correct age-related changes in the elderly. The invention may also be used in the cosmetic industry for producing active ingredients for enhancing regeneration and improving the scalp, face and body, in particular for the manufacture of active additives to combat deep wrinkles, removal of skin defects, stimulation and acceleration of hair growth, controlling hirsutism, etc.

### RELATED APPLICATIONS

This application claims the benefit under 35 U.S.C. §119(a) of Russian Patent Application No. 2014126089, filed Jun. 26, 2014, and entitled “Substance and Method for Modulating Proliferation and Differentiation of Regulatory, Stem, and Other Somatic Cells”, the entire contents of which are incorporated herein by reference in their entirety.

### FIELD OF THE DISCLOSURE

The present disclosure relates to the fields of molecular biology and regenerative medicine, and in particular, methods of modulating cell proliferation and/or differentiation, particularly, in mammals.

### BACKGROUND

Cell transplantation therapies have been plagued with safety concerns focused on the potential risk of unwanted activation of the host immune system. Therefore, there is a long felt need for a biologic therapy that provides a functional recovery for the host while avoiding activation of the immune system.

### SUMMARY

The present disclosure provides compositions and methods or uses for those compositions to prevent, treat, or cure disease. Moreover, the compositions and methods of the disclosure may be used to improve a sign or symptom of a disease.

Compositions and methods of the disclosure can replace conventional cell therapy. Conventional cell therapies present risks to the recipient's health, often as a result of unwanted activation of his immune system and graft rejection. Compositions and methods of the disclosure include cell-free extracts from one or more cell types. In some embodiments, compositions include RNA preparations (e.g., purified RNA preparations), for example total RNA preparations (e.g., purified preparations of total RNA) isolated from one or more somatic cell types (for example, regulatory lymphoid cells and/or stem cells). Because they are non-immunogenic, compositions and methods of the disclosure eliminate a need for personalized umbilical cord blood cell banking.

Compositions and methods of the disclosure can replace blood transfusion therapy. Blood transfusion therapies present risks (e.g., contracting an infectious disease or hemolytic transfusion reaction) to the recipient's health.

Conventional therapies merely address symptoms rather than the cause of the disease. Compositions of the disclosure structurally and functionally repair or restore damaged tissues. Thus, compositions and methods provided by the disclosure may prevent or cure diseases.

Compositions and methods of the disclosure are useful for modulating dysregulated proliferation and/or differentiation of cells, particularly, for example, in mammals. More specifically, in some embodiments compositions and methods of the disclosure are used to modulate the population size and differentiation of various cells (for example, mammalian cells) by activating and/or normalizing the regulatory function of lymphoid cells. In some embodiments, compositions include one or more RNA preparations (e.g., purified total RNA preparations) derived from lymphoid cells of the spleen, thymus, lymph nodes, from peripheral blood lymphocytes, from bone marrow, or stem cells (e.g., from cord blood, umbilical cord, and/or placenta) of healthy donors, which preparation(s) restore normal function to a tissue or cell population of a host, to treat, ameliorate, or prevent a disease, disorder, or condition associated with a dysregulation of cell proliferation and/or differentiation.

In some embodiments, compositions and methods of the disclosure are useful in particular for the activation of stem cells, and further modulate their stimulatory action. In some embodiments, compositions and methods of the disclosure are useful for treating or preventing hematopoietic, blood, degenerative, hyperproliferative, or autoimmune diseases, disorders, or conditions. In some embodiments, compositions and methods of the disclosure are useful for correcting a number of hereditary and congenital defects.

In some embodiments, compositions and methods of the disclosure are useful for modulating proliferation and/or differentiation of various mammalian cells in vitro or in vivo. In some embodiments, compositions include total RNA preparations (e.g., purified total RNA preparations) derived from one or more organs, tissues, or somatic cells. In some embodiments, RNA preparations (e.g., total RNA preparations) are purified from their natural environment. In some embodiments, total RNA preparations are obtained as preparations of total RNA from cells without performing a sequence selection or size selection. In some embodiments, a regulatory RNA preparation includes a total RNA preparation. In some embodiments, a regulatory RNA preparation is a regulatory RNA preparation prepared from total RNA (e.g., isolated or purified from total RNA). These compositions may be useful as additional impact in the treatment and/or prevention of hematopoietic, blood, degenerative, tumor, and autoimmune diseases, disorders, and conditions and in correction of certain hereditary and congenital defects via their compensation.

The compositions and methods of the disclosure take advantage of morphogenetic activity of lymphoid cells to control pathological processes in the body, particularly, in the mammalian body, and, preferably, human body while avoiding complications of unwanted activation of the host immune system or conducting a laborious search for the best compatible donor. In some embodiments, compositions and methods of the disclosure induce cell proliferation and regeneration in the body, particularly, in the mammal, and, preferably, in the human body. In some embodiments, compositions of the disclosure include one or more RNA molecules of a total RNA preparation (e.g., a purified total RNA preparation) derived from one or more cells or cell types.

Compositions of the disclosure may be “isolated”, “extracted”, or “derived” from cell populations. Following the isolation, extraction, or derivation of RNA and/or total RNA from these cells, the resultant composition or preparation may be manipulated by, for example, purifying RNA molecules, concentrating RNA molecules, modifying RNA molecules, and/or combining RNA molecules (e.g., with one or more other agents), such that the resultant composition is not found in nature. Furthermore, the resultant non-natural isolated, extracted, or derived compositions or preparations provide superior and unexpected properties compared to RNA populations found in nature. For example, compositions and preparations described by the disclosure may include amounts and/or concentrations of RNA molecules (e.g., including RNA preparations from one cell type or combinations of RNA preparations from different cell types) effective for modulating the population size and differentiation of various cells (for example, mammalian cells) by activating and/or normalizing the regulatory function of cells (e.g., lymphoid cells) after administration (e.g., injection) to a subject.

In some embodiments, compositions may further comprise one or more of the following: a buffer (e.g., tris buffer, bicarbonate buffer, phosphate buffer, MOPS buffer, etc.), an RNAse inhibitor (e.g., an inhibitor of RNAase A, RNAse B, RNAse C, etc.), a preservative (e.g., one or more salts, chelating agents, detergents, and/or antimicrobial agents, or a combination thereof), a protectant (e.g., a cryoprotectant), and/or a pharmaceutically acceptable excipient. In some embodiments, the composition comprises modified RNA (e.g., methylated RNA or unmethylated RNA, phosphorolyated or dephosphorylated RNA, etc.). In some embodiments, compositions are lyophilized or frozen.

In some embodiments, RNA preparations (e.g., total RNA preparations) extracted from cells (e.g., lymphoid cells) possess morphogenetic activity similar to the activity of these cells (e.g., lymphoid cells) themselves. As used herein, the “morphogenetic” activity of cells (e.g., lymphoid cells) refers to regulatory activity consisting of exercising control of various cell type proliferation and/or differentiation. Data provided in this disclosure demonstrate morphogenetic activity for total RNA preparations from lymphoid cells of the spleen or thymus, peripheral blood lymphocytes, and bone marrow. Moreover, the disclosure demonstrates that total RNA preparations isolated from cord blood as well as from umbilical cord or placenta provide effects, similar to the effects provided by intact cord blood cells, umbilical cord or placenta themselves. The term “intact” lymphoid cells refers to non-activated lymphoid cells. Total RNA preparations derived from any other type of somatic cell provide effects similar to that provided by the cells of the same type themselves. That is, RNA-containing preparations derived from any other type of somatic cell are efficient in the increasing functional activity and regeneration ability of homologous tissues, as well as in their trophic function with refilling deficiency of endogenous RNA.

In some embodiments, compositions and methods of the disclosure use the morphogenetic properties of RNA preparations (e.g., total RNA preparations) from cells (e.g., lymphoid cells) to modulate cell proliferative activity in various organs and tissues. In some embodiments, compositions, methods, and uses described herein are widely applicable in veterinary and human medicine because they make it possible to replace or augment the function of regulatory cells with their functional analog in the form of non-immunogenic RNA preparations (e.g., total RNA preparations). Thus, compositions and methods of the disclosure employ a non-immunogenic means to transfer proliferative or anti-proliferative signals. As used herein, the term “non-immunogenic” is meant an absence of limitations related to individual- or species-specific antigenicity for RNA (see also Russian Patent No. 2314814). For example, compositions described herein allow successful xenogeneic transfer of total RNA (e.g., purified total RNA) from lymphoid cells (see Example 7). Because the RNA preparations (e.g., total RNA preparations) of the disclosure are non-immunogenic ones, any allogeneic and xenogeneic RNA preparations (e.g., total RNA preparations) can be administered, without restriction, to the mammalian and, in particular, human body. Regulatory RNA preparations (e.g., total RNA preparations) isolated from cells (e.g., lymphoid cells) and organs (e.g., lymphoid organs) of healthy donors differ in that they not only exert a correcting effect on target somatic cells, but they also restore the altered regulatory function of the recipient's lymphoid cell system in a variety of pathological conditions.

In some embodiments, compositions and methods of the disclosure include variants of regulatory total RNA preparations isolated from lymphoid cells of the spleen or thymus, peripheral blood lymphocytes, lymphatic nodes, or bone marrow of a healthy donor, or, alternatively or additionally, from lymphoid cells of the spleen or thymus, peripheral blood lymphocytes, lymphatic nodes, or bone marrow of a healthy donor treated to activate T-cells of the immune system at a point in time when these T-cells exert their stimulating (helper) or suppressing activity toward somatic cells (for example, somatic cells of a particular cell type, e.g., histotype).

Functional differences among regulatory total RNA preparations of the disclosure are determined by the qualitative differences in a functional state among the initial donor cells harvested under normal conditions or the donor cells harvested at various stages of realization of their morphogenetic function. In some embodiments, RNA preparations (e.g., total RNA preparations) are purified from their natural environment.

In some embodiments, the compositions and methods of the disclosure include variants of total RNA preparations derived from cord blood cells or whole cord blood, umbilical cord cells or whole umbilical cord, or placenta, of a healthy intact donor.

In some embodiments, compositions and methods of the disclosure include total RNA preparations isolated from any type of mammalian cell. In some embodiments, total RNA refers to RNA that has been isolated in a non-selective manner (e.g., in a manner that does not enrich any particular subpopulation of RNA, such as pre-mRNA, mRNA, and miRNA). The mammalian cell may be a cell type which is required for restoring the tissue structure and function. Because cell transplantation is associated with possible adverse effects and requires preliminary immunosuppression for the recipient, it involves risks to the patient's health, and even survival. Compositions and methods of the disclosure make it possible to avoid graft-versus-host reactions and obviate the need of immunosuppression of the host immune system to prevent donor cell rejection. The functional recovery demonstrated by the recipient body following administration of compositions of the disclosure comprising total RNA preparations derived from intact or preliminarily activated bone marrow of donor rats are comparable to the functional recovery resulting from a bone marrow transplant (see Example 6).

In some embodiments, compositions and methods of the disclosure include a regulatory total RNA preparation isolated (e.g., purified) from lymphoid cells or lymphoid organs of a donor, which may optionally contain a population of activated (stimulating or suppressing) T-cells generated in response to activation of the donor immune system. Activation of T-cells of a healthy donor may be performed in vivo, ex vivo, or in vitro. In some embodiments, regulatory total RNA is isolated in vitro, preferably, from a population of donor cells (e.g. lymphoid cells of the spleen, thymus, lymph nodes, peripheral blood lymphocytes, or bone marrow) including at least one activated T-cell, by the standard method using the Trizol reagent and phenol-chloroform extraction (Chomczynski P. BioTechniques, 1993, vol. 15, pp. 532-537). Preferably, isolation of RNA (e.g., total RNA) is performed at a time when the immune cells manifest their stimulating or suppressing effect on cells of a particular cell type(s) (e.g., histotype(s)), yielding a regulatory total RNA preparation that possess, respectively, stimulating or suppressing activity toward the same cells of the host. In some embodiments, RNA preparations are isolated and/or purified under sterile conditions.

In an embodiment of the disclosure, compositions and methods of the disclosure include regulatory total RNA preparations. In some embodiments, regulatory total RNA preparations are preparations of total RNA isolated from intact or activated lymphoid cells of the spleen, thymus, lymph nodes, peripheral blood lymphocytes, or bone marrow of a healthy donor. In some embodiments, regulatory preparations may further comprise one or more of the following: a buffer (e.g., tris buffer, bicarbonate buffer, phosphate buffer, MOPS buffer, etc.), an RNAse inhibitor (e.g., an inhibitor of RNAase A, inhibitor of RNAse B, inhibitor of RNAse C, etc.), a preservative (e.g., one or more salts, chelating agents, detergents, and/or antimicrobial agents, or a combination thereof), a protectant (e.g., a cryoprotectant), and/or a pharmaceutically acceptable excipient. In some embodiments, regulatory preparations are lyophilized or frozen.

Total RNA preparations may be also isolated from any other tissue or any other somatic cell of a healthy donor. In particular, total RNA preparations may be isolated from any stem cell of a healthy donor, including bone marrow cells, umbilical cord cells (including whole cord blood and Wharton's Jelly (substantia gelatinea funiculi umbilicalis)), and placenta. The term “stem cells” refers to cells that are the progenitors of somatic cells, having a high proliferative potential and totipotency (i.e., the ability to differentiate into any somatic cells of a body). The term “somatic cells” refers to all body cells except germ cells.

In some embodiments, compositions and methods described herein modulate cell proliferation and/or differentiation, particularly, mammalian cell proliferation and/or differentiation. In one embodiment, a composition comprising a total RNA preparation or portion thereof isolated from lymphoid cells and/or bone marrow of a healthy donor under normal conditions is administered to a subject, particularly, to a mammal (preferably, a human). Alternatively, or in addition, a composition comprising a total RNA preparation or portion thereof derived from lymphoid cells and/or bone marrow from a healthy donor under activated conditions (at the time when the original cells manifest, in vivo or in vitro, their stimulating (from about 15 minutes to about 48 hours after activation, depending on the target tissue) or suppressing (from about 48 hours to about 96 hours or more after activation, depending on the target tissue) activity towards cells of a particular histotype), is administered to a subject, particularly, to a mammal (preferably, a human).

In some embodiments, compositions and methods of the disclosure modulate mammalian cell proliferation and/or differentiation and are useful in treating or preventing hematopoietic, blood, degenerative, tumor, and autoimmune diseases, disorders, and conditions and to correct certain hereditary, congenital or age-related defects.

In some embodiments, compositions comprise a combination of total RNA preparations derived from various organs or somatic cells. In some embodiments, such combinations are useful for treating or preventing hematopoietic, blood, degenerative, tumor, and autoimmune diseases, disorders, and conditions, and to correct (e.g., eliminate) a number of hereditary, congenital, or age-related defects, including, but not limited to, osteopetrosis, cerebral palsy, vision and hearing disorders (deafness). In some embodiments, compositions include a total RNA preparation (e.g., a purified total RNA preparation) derived from a lymphoid cells and/or bone marrow, and a total RNA preparation derived from a different organ, tissue, somatic cell, particularly, from stem cell.

In some embodiments, compositions and methods of the disclosure include a stimulating preparation (e.g., a RNA or total RNA preparation derived from stimulated or activated lymphoid or bone marrow cells). Stimulating preparations can be used to modulate the morphogenetic function of lymphocytes to affect cells of various tissues of the recipient's body (e.g., a mammalian body).

In some embodiments, compositions and methods of the disclosure include a suppressing preparation (e.g., an RNA or total RNA preparation derived from stimulated, or activated immune cells, e.g. lymphoid or bone marrow cells). Suppressing preparations can be used to modulate the morphogenetic function of lymphocytes to affect cells of various tissues of the recipient's body (e.g., a mammalian body).

In some embodiments, compositions and methods of the disclosure include the use of total RNA preparations according to the invention as a replacement for blood transfusion to a subject.

In some embodiments, compositions and methods of the disclosure include the use of total RNA preparations according to the invention as a replacement for stem cell therapy in a subject.

In some embodiments, compositions and methods of the disclosure include the use of total RNA preparations according to the invention as a replacement for one or more bone marrow transplant(s) to a subject.

In some embodiments, compositions and methods of the disclosure include, but are not limited to, a total RNA preparation derived from any intact cell of healthy donor (e.g., not subjected to activation of T-cell population), and/or a regulatory total RNA preparation derived from lymphoid cells or organs of healthy donor treated to activate a T-cell population of an immune system. Donor cells of the disclosure may be derived from any vertebrate species.

Donor cells of the disclosure may be derived from any healthy mammalian species, preferably from bovine animals. Alternatively, donor cells of the disclosure may be derived from any non-mammalian vertebrate species. Donor cells of the disclosure may be derived from one or more tissues of a human donor. Human donors may be male or female of any age. Preferably, tissues and/or cells derived from young healthy donors are used to obtain RNA preparations of the disclosure. Young, healthy donors are typically male or female subjects between the ages of 18 years and 50 years that have no patent or latent (e.g., underlying) medical conditions, and/or do not exhibit signs or symptoms of disease or infection (e.g., chronic disease or acute disease). In some embodiments, young healthy donors are between the ages of about 18 years and about 25 years. In some embodiments, young healthy donors are between the ages of about 18 years and 30 years. In some embodiments, young healthy donors are between the ages of 18 years and 40 years. However, donors can be younger or older males or females in some cases. In some embodiments, a healthy donor is a donor that does not have the condition or disease that will be treated in a subject by the administration of a composition described by the disclosure. For example, if a subject has an autoimmune disease (e.g., rheumatoid arthritis), a donor cells are derived from a healthy donor that does not have an autoimmune disease.

In some embodiments, compositions and methods of the disclosure include a RNA preparation (e.g., a total RNA preparation) that is any combination of one or more RNA preparations (e.g., total RNA preparations) selected from the group comprising regulatory (e.g., total) RNA preparation(s) isolated from lymphoid cells of the spleen, thymus, lymph nodes, from peripheral blood lymphocytes, and from bone marrow of an intact healthy donor and/or healthy donor treated to activate a T-cell population of the immune system, the isolation being performed at the time when the cells express stimulating or suppressing activity towards cells of the same or another cell type (e.g., histotype).

In some embodiments, compositions and methods of the disclosure modulate mammalian cell proliferation and/or differentiation to treat or prevent immunodeficiency. Exemplary immunodeficiency conditions, include, but are not limited to, immunodeficiencies in which there are signs of autoimmune processes, such as ataxia-telangiectasia; thymoma; sex-linked hypogammaglobulinemia; immunodeficiencies with hyper IgM; IgA deficiency; Nezelof and Wiskott-Aldrich syndromes; atrophic gastritis; Myasthenia gravis; Pemphigus vulgaris; encephalomyelitis; collagenoses; systemic lupus erythematosus; rheumatoid arthritis; Sjogren's syndrome; ulcerative colitis; Evans syndrome; immune thyroiditis; diabetes type 1 and type 2; the immune thrombocytopenia; cold agglutinin disease; paroxysmal cold hemoglobinuria; hyperthyroidism; infertility caused by disordered immune mechanisms; sympathetic ophthalmia; chronic active hepatitis; coagulopathy due to impaired synthesis of antibodies; primary biliary cirrhosis; phacogenic uveitis; idiopathic Addison's disease, postvaccinal encephalitis; idiopathic hypoparathyroidism; periarteritis nodosa; dermato- or polymyositis; scleroderma; and multiple sclerosis.

In some embodiments, compositions and methods of the disclosure modulate mammalian cell proliferation and/or differentiation to treat or prevent a hematological disease or disorder in said mammal. Hematological diseases or disorders of the disclosure include, but are not limited to, anemia of any etiology (including inherited forms of anemia), such as for example posthemorrhagic anemia, hemolytic anemia, Mediterranean anemia (thalassemia), hypo- and aplastic anemia, iron deficiency anemia, vitamin B 12 deficiency anemia, folic acid deficiency anemia, anemia of mixed origin, hemophilia. In certain aspects, the disclosure provides compositions and methods to modulate mammalian cell proliferation and/or differentiation to treat or prevent anemia by replacing the current treatment (e.g. blood transfusion). RNA preparations and total RNA preparations of the disclosure increase the number of erythrocytes and hemoglobin levels in both healthy and anemic individuals.

Despite remarkable progress in the field of blood transfusion, it is becoming increasingly evident that effective treatment of hematologic disorders and diseases requires regeneration of hematopoietic tissue of the recipient. Therapeutic efficacy of a blood transfusion is limited by the lifetime of transfused blood. In contrast to the limitations of blood transfusion technology, compositions and methods of the disclosure stimulate regeneration of hematopoietic tissue of the patient for a significantly longer duration.

In some embodiments, compositions and methods of the disclosure include RNA preparations isolated from lymphoid cells, bone marrow, cord blood, umbilical cord and/or placenta can be used to treat hematological diseases and disorders characterized by impaired proliferative processes, disturbance of cell differentiation in the bone marrow, damage to cell or tissue membranes, or functional disorders of cells. Exemplary hematological diseases and disorders include, but are not limited to, acute or chronic hemorrhagic anemia, inherited or acquired dyserythropoietic anemia, anemia characterized by impaired production of erythropoietin or appearance of erythropoietin inhibitors, autoimmune anemia, pancytopenia; hemolytic anemia arising from splenomegaly, heavy metal or acids poisoning; congenital anemias associated with impaired synthesis of hemoglobin chains (sickle cell anemia, thalassemia); hereditary hemolytic anemias associated with impaired erythrocyte membrane (hereditary microspherocytosis, hereditary elliptocytosis, hereditary stomatocytosis, hereditary acanthocytosis, anemia associated with reduced amounts of polyunsaturated fatty acids of the membrane), hereditary hemolytic anemias associated with impaired enzyme activity of red blood cells; congenital megaloblastic anemia associated with impaired synthesis of DNA and RNA (including the syndrome of Rogers, accompanied by deafness, diabetes mellitus, and megaloblastic anemia), symptomatic anemia in patients with myelofibrosis, chronic lymphocytic leukemia, infectious mononucleosis, hematosarcoma, chronic hepatitis, thymoma, chronic myeloid leukemia, Hodgkin's disease, systemic lupus erythematosus; symptomatic anemia associated with inhibition of proliferation of bone marrow cells after exposure to toxic or drugs, cytotoxic drugs or ionizing radiation; and congenital or acquired thrombocytopathia and acute hemorrhagic vasculitis.

In some embodiments, compositions and methods of the disclosure may be used to treat diseases associated with impaired blood flow to the microvasculature. Exemplary diseases associated with impaired blood flow to the microvasculature include, but are not limited to, arteritis obliterans, ischemic heart disease, atherosclerosis, diabetes mellitus type 1 and type 2, crush syndrome, and regeneration of muscle tissue after prolonged immobilization of limbs. Administration of RNA preparations isolated from the spleen of intact or anemic animals significantly increase blood circulation in multiple organs including liver, spleen, pancreas, kidney (see Example 9).

In some embodiments, compositions and methods of the disclosure may be used to treat or prevent of radiation damage, radiation sickness, or atomic disease in mammal. Moreover, compositions and methods of the disclosure may be used to treat or prevent a side effect of radiation therapy, for example, in a cancer patient.

In some embodiments, compositions and methods of the disclosure may be used to treat or prevent a side effect of chemotherapy.

In some embodiments, compositions and methods of the disclosure may be used to reduce or reverse a sign(s) or a symptom(s) of aging. Exemplary signs or symptoms of aging include, but are not limited to, fatigue, vision impairment or degeneration, cataract, glaucoma, retinal degeneration, auditory impairment, hair cell degeneration, cardiovascular disease, bleeding and/or clotting disorders, clotting or damage to vasculature, stroke, neurological impairment, neuromuscular impairment, cognitive impairment including memory loss and motor impairment, muscular degeneration including loss of muscle mass, metabolic disease including diabetes, inflammatory disease including arthritis, autoimmune disease, organ failure (including impairment or failure of the kidneys and/or liver), incontinence, respiratory impairment, loss of taste or olfactory sensitivity or function, digestive disorders, cancer, hyperproliferative disorders, bone and/or cartilage degeneration including osteoporosis and osteoarthritis, skin-related disorders, immune system disorders, impairment of wound healing, infection, hair loss, and impaired mobility.

In some embodiments, the method of modulation of proliferation and/or differentiation of mammalian cells is a method for the prophylaxis or treatment of chemical lesion of the bone marrow in the mammal.

In some embodiments, compositions and methods of the disclosure may be used to treat or prevent chemical destruction of bone marrow cells.

Disorders, diseases and/or conditions associated with dysregulation of cell proliferation and/or differentiation of the disclosure include, but are not limited to, autoimmune disorders and diseases (e.g., autoimmune hemolytic anemia, autoimmune thrombocytopenic purpura, Graves' disease (toxic diffuse goiter), Goodpasture's syndrome (hemorrhagic pulmonary-renal syndrome, systemic capillaritis, hereditary), Hashimoto's thyroiditis, and multiple sclerosis).

Disorders, diseases and/or conditions associated with dysregulation of cell proliferation and/or differentiation of the disclosure include, but are not limited to, degenerative diseases (e.g., Alzheimer's disease, Parkinson's disease, and amyloidosis).

Disorders, diseases and/or conditions associated with dysregulation of cell proliferation and/or differentiation of the disclosure include, but are not limited to, hyperproliferative or tumor diseases (e.g., prostate adenoma and prostate cancer, benign and malignant breast tumor).

Disorders, diseases and/or conditions associated with dysregulation of cell proliferation and/or differentiation of the disclosure include, but are not limited to, neuro-endocrine disorders (e.g., polycystic ovaries; blood diseases and violations of blood).

Disorders, diseases and/or conditions associated with dysregulation of cell proliferation and/or differentiation of the disclosure include, but are not limited to, hereditary diseases and defects associated with impaired regulation of cell proliferation or differentiation (e.g., osteopetrosis; Cerebral Palsy; and Hearing disorders. Exemplary hearing disorders may be characterized by diminished hearing, neuro-sensory hearing loss, age-related hearing loss, and deafness (including congenital deafness).

Disorders, diseases and/or conditions associated with impaired cell proliferation and/or differentiation include, but are not limited to, wound healing, psoriasis, cervical erosion, periodontal disease, alveolitis, gingivitis, atherosclerosis, benign tumors, malignant tumors, tumors resistant to chemotherapy; conditions requiring enhanced regeneration, such as bone fractures, burns, ulcers, hypertrophic scars, torn ligaments, soft tissue and internal organ injuries, and skin flap engraftment.

In some embodiments, compositions and methods of the disclosure may be used to treat or prevent excessive cell proliferation. Alternatively, excessive cellular proliferation may be caused by modulation of cellular differentiation. Compositions and methods of the disclosure may prevent or inhibit metastasis of malignant cells. Exemplary conditions characterized by excessive cell proliferation include, but are not limited to, cancer, benign tumor and hyperproliferative disorders (e.g., scar formation, psoriasis, and atherosclerosis).

In an embodiment of the disclosure, compositions and methods of the disclosure may be used to modulate cell proliferation and/or differentiation for all types of benign and malignant tumors, including, those tumors resistant to chemotherapy or radiotherapy.

In some embodiments, compositions and methods of the disclosure may be used to modulate cell proliferation and/or differentiation by restoring normal and/or healthy levels of cell proliferation and/or differentiation in a subject having impaired cell proliferation and/or differentiation.

The disclosure provides a pharmaceutical composition to treat a disorder, disease, or condition associated with distorted cell proliferation and/or differentiation in the subject's body, particularly, mammalian body, wherein the composition includes an effective amount of any RNA preparation or variant thereof described herein and a pharmaceutically acceptable carrier, diluent, or excipient.

The disclosure provides a pharmaceutical composition to restore normal function that is compromised under disease conditions, e.g., aberrant cell proliferation and/or differentiation in the mammalian body, wherein the composition includes an effective amount of any RNA preparation or variant thereof described herein and a pharmaceutically acceptable carrier, diluent, or excipient. Exemplary disease conditions include, but are not limited to, degenerative conditions, hyperproliferative conditions (tumor), and autoimmune conditions.

The disclosure provides a pharmaceutical composition to treat a disorder, disease, or condition associated with distorted cell proliferation and/or differentiation in the subject's body, particularly, mammalian body, wherein the composition includes an effective amount of any regulatory total RNA preparation or variant thereof derived from a lymphoid and/or bone marrow cell and, optionally, a total RNA preparation isolated one or more cells of another histotype with a pharmaceutically acceptable carrier, diluent, or excipient.

The compositions and methods of treatment provided by the invention can be used in the practice of medicine and in veterinary medicine, e.g., for the treatment of agricultural animals, domestic and working animals, as well as pets, including dogs, cats, rodents, and birds.

### BRIEF DESCRIPTION OF THE FIGURES

FIG. 1 is a photograph depicting an erythropoiesis reconstruction in an EI (erythroblastic islet) culture (1 IU/ml of erythropoietin, 24 h of cultivation). The photograph shows involuting EIs; the “crown” of one of them contains two proerythroblasts.

FIG. 2 is a photograph depicting a typical pattern of EI development in vitro (0.5 IU/ml of erythropoietin, 24 h of cultivation). The photograph shows two EIs of maturation class 3 whose “crowns” contains erythroid cells at different stages of maturation.

FIG. 3 is a photograph depicting the maturation of erythroid cells in an EI culture (1.5 IU/ml of erythropoietin, 96 h of cultivation). On the left, an involuting EI whose “crown” entirely consists of reticulocytes.

### DETAILED DESCRIPTION

Being an essential and phylogenetically more ancient functional part of the immune system than the one that ensures the development of humoral immunity and antibody formation, morphogenetic function of lymphocytes is responsible for the regulation of proliferative processes in the body. Normally regulation involves timely stimulation and timely inhibition of proliferation of cells of any tissue of the body, thus ensuring the constancy of number of cells and anatomical integrity of all organs and tissues in the process of growth and in the process of physiological and reparative regeneration. Morphogenetic function of lymphoid cells is provided by implementing a two-stage (two-phase) program of regulation a proliferation and differentiation of cells of target tissues, and at the same time it is a constant component of immune responses as well as provides a proliferation of immunocompetent cells for both humoral and cellular immunity.

The present invention allows to obtain tissue-specific regulatory preparations of directed action, corrective, stimulating and inhibiting the processes of cell division and differentiation in various pathological conditions—means that can be used in medical practice in the field of hematology, blood transfusion, surgery, oncology, radiology, gynecology, in the treatment of degenerative, autoimmune, hyperproliferative disorders, diseases and conditions, particularly tumoral diseases, as well as a number of hereditary and inherent diseases and defects.

The preparations according to the invention are obtained by isolation from lymphoid cells of the spleen, thymus, lymph nodes, from bone marrow, from peripheral blood lymphocytes of healthy mammals, particularly from donated human blood, umbilical cord blood and/or umbilical cord stromal cells, whole umbilical cord, and from placenta a total RNA fraction, the different nature of action of which is determined by source of its production and/or variation of the functional state of the source of lymphoid cells in normal conditions and at different stages of manifestation of their morphogenetic function.

Compositions of the disclosure may be “isolated”, “extracted”, or “derived” from cell populations. Following the isolation, extraction, or derivation of RNA or total RNA from these cells, the resultant composition or preparation may be manipulated by concentrating RNA molecules, modifying RNA molecules, or combining RNA molecules such that the resultant composition is not found in nature. Furthermore, the resultant non-natural isolated, extracted, or derived compositions or preparations provide superior and unexpected properties compared to RNA populations found in nature. For example, compositions and preparations described by the disclosure may include amounts of RNA molecules effective for modulating the population size and differentiation of various cells (for example, mammalian cells) by activating and/or normalizing the regulatory function of lymphoid cells in a subject.

An “amount of RNA molecules effective for modulating”, or “effective amount”, refers to an amount or concentration of RNA molecules sufficient for activating and/or normalizing the regulatory function of lymphoid cells in a subject. The effective amount can be an unnatural amount or concentration of RNA (e.g., an amount or concentration of RNA molecules that is significantly higher than found in nature, for example than found naturally in a biological sample such as a cell or cellular sample). The effective amount can be an amount that is administered to a subject in a single dose or in multiple doses as part of a treatment for a disease or condition.

In some embodiments, compositions may further comprise one or more additives selected from the following: a buffer (e.g., tris buffer, bicarbonate buffer, phosphate buffer, etc.), an RNAse inhibitor (e.g., an inhibitor of RNAase A, RNAse B, RNAse C, etc.), a preservative (e.g., one or more salts, chelating agents, detergents, and/or antimicrobial agents, or a combination thereof), a protectant (e.g., a cryoprotectant), a stabilizing agent, a solubilizing agent, and/or a pharmaceutically acceptable excipient (e.g., a pharmaceutically acceptable salt). Additives may be present in the composition at any suitable concentration (e.g., a concentration that allows the RNA preparation to function as described by the disclosure). In some embodiments, the buffer is a non-natural buffer (for example not a bicarbonate buffer). In some embodiments, the buffer is present at a concentration of at least 1 mM, 5 mM, 10 mM, 25 mM, 50 mM, or 100 mM.

Buffers may be present at a concentration that maintains compositions at a physiologically relevant pH (e.g., between about pH 5.5 and about pH 8.0). In some embodiments, buffers are present at a concentration that maintains compositions at a pH between about pH 6.0 and about 7.0. In some embodiments, buffer is present at a concentration that maintains compositions at a pH between about pH 6.8 and about pH 7.5.

RNAse inhibitors may be present at a concentration that prevents degradation of RNA. For example, RNAase inhibitors can be present at a concentration between about 1 U/μL to about 50 U/μL. In some embodiments, compositions described herein further comprise RNAse inhibitors at a concentration of less than 1 U/μL. In some embodiments, compositions described herein further comprise RNAse inhibitors at a concentration of about 1 U/μL, about 5 U/μL, about 10 U/μL, about 25 U/μL, or about 50 U/μL. In some embodiments, an RNase inhibitor is a protein, protein fragment, peptide or small molecule which inhibits the activity of any or all of the known RNAses, including RNase A, RNase B, RNase C, RNase T1, RNase H, RNase P, RNAse I and/or RNAse III. Non-limiting examples of RNase inhibitors include ScriptGuard (Epicentre Biotechnologies, Madison, Wis.), Superase-in (Ambion, Austin, Tex.), Stop RNase Inhibitor (5 PRIME Inc, Gaithersburg, Md.), ANTI-RNase (Ambion), RNase Inhibitor (Cloned) (Ambion), RNaseOUT™ (Invitrogen, Carlsbad, Calif.), Ribonuclease Inhib III (Invitrogen), RNasin® (Promega, Madison, Wis.), Protector RNase Inhibitor (Roche Applied Science, Indianapolis, Ind.), Placental RNase Inhibitor (USB, Cleveland, Ohio) and ProtectRNA™ (Sigma, St Louis, Mo.).

In some embodiments, compositions described herein further comprise a preservative (e.g., one or more salts, chelating agents, detergents, and/or antimicrobial agents, or a combination thereof) and/or a protectant (e.g., a cryoprotectant). Non-limiting examples of salts include ammonium, potassium, and sodium salts (e.g., ammonium sulfate, sodium chloride, sodium citrate, potassium chloride, etc.). Non-limiting examples of chelating agents include EDTA and EGTA. Non-limiting examples of protectants include dimethyl sulfoxide (DMSO), ethylene glycol, propylene glycol, sucrose, glycerol, other suitable sugars and alcohols (e.g., polyols), or other suitable protectants. In some embodiments, compositions are lyophilized or frozen.

Generally, addition of one or more of the foregoing components stabilizes and/or prevents the degradation of RNA. In some embodiments, compositions described herein include one or more additives (e.g., a buffer, an RNAase inhibitor, a preservative, a protectant, and/or pharmaceutically acceptable excipient), wherein at least one additive is present in an amount such that the composition has no naturally-occurring counterpart.

In some embodiments, RNA preparations (e.g., purified total RNA preparations) and compositions comprising RNA preparations are provided in a suitable container. Examples of suitable containers include, but are not limited to, syringes, vials, tubes, bottles, flasks, blister packs, etc. Containers can be made of glass, plastic, polymers, or any other suitable material. In some embodiments, containers are sterilized. In some embodiments, compositions are sterilized (e.g., treated with an antimicrobial agent).

The invention relates to a method of modulation of cell proliferation and/or differentiation in a subject by administering to the subject the preparations (means) according to the invention. Wherein said method of modulation may be used to treat conditions, diseases or disorders, not limited to, but selected from the group comprising rheumatoid arthritis, Lyme arthritis, systemic lupus erythematosus, Crohn's disease, ulcerative colitis, inflammatory bowel disease, insulin dependent diabetes mellitus, insulin independent diabetes mellitus, thyroiditis, asthma, allergic diseases, psoriasis, dermatitis, scleroderma, graft versus host disease, graft rejection of any organ or tissue, sarcoidosis, atherosclerosis, disseminated intravascular coagulation, Kawasaki's disease, Grave's disease, nephrotic syndrome, chronic fatigue syndrome, Wegener's granulomatosis, Henoch-Schoenlein purpurea, chronic active hepatitis, cachexia, acquired immunodeficiency syndrome, Huntington's chorea, Parkinson's disease, Alzheimer's disease, stroke, hemolytic anemia, malignancies, heart failure, myocardial infarction, alopecia, arthropathy of any etiology, arteriosclerosis, atopic allergy, autoimmune bullous disease, pernicious anemia, all types of hepatitis, Acquired Immunodeficiency Disease Syndrome, overall transient immunodeficiency, cardiomyopathy, female infertility, premature ovarian failure, fibrotic lung disease, interstitial lung disease, Sjogren's disease, fibrosis, hypoparathyroidism, acute and chronic immune disease associated with organ transplantation, osteoarthrosis, idiopathic leucopenia, autoimmune neutropenia, glomerulonephritis, Lyme disease, idiopathic male infertility, sperm autoimmunity, multiple sclerosis, sympathetic ophthalmia, Goodpasture's syndrome, spondylitis, Still's disease, systemic sclerosis, Sjogren's syndrome, Takayasu's disease, autoimmune thrombocytopenia of any etiology, autoimmune thyroid disease, hyperthyroidism, hypothyroidism of any etiology, phacogenic uveitis, primary vasculitis, vitiligo, chronic liver diseases, liver cirrhosis, mental disorders (e.g., depression and schizophrenia), cancers such as lung, breast, stomach, bladder, colon, colorectal carcinoma, pancreas, ovarian, prostate and rectal cancer; allergic rhinitis, allograft and/or xenograft rejection, amyotrophic lateral sclerosis, anemia, angina pectoris, arterial hypertension, B cell lymphoma, bone marrow transplant (BMT) rejection, Burkitt's lymphoma, cardiomyopathy, chemotherapy associated disorders, chronic myelocytic leukemia (CML), chronic alcoholism, chronic inflammatory processes, chronic lymphocytic leukemia (CLL), chronic obstructive pulmonary disease (COPD), congestive heart failure, conjunctivitis, contact dermatitis, coronary artery disease, Creutzfeldt-Jakob disease, cystic fibrosis, demyelinating diseases, dermatologic conditions, diabetic arteriosclerotic disease, Down's Syndrome in young and middle age, eczema, encephalomyelitis, endocarditis, endocrinopathy, extrapyramidal and cerebellar disorders, Friedreich's ataxia, functional peripheral arterial disorders, gastric ulcer, glomerular nephritis, thrombolytic thrombocytopenic purpura, hemorrhage, Hodgkin's disease, asthenia, stroke, spinal muscular atrophy, Kaposi's sarcoma, leprosy, lipedema, lymphedema, malignant Lymphoma, malignant histiocytosis, malignant melanoma, multiple myeloma, myelodyplastic syndrome, nephrosis, neurodegenerative diseases, non-hodgkin's lymphoma, organomegaly, osteoporosis, peripheral atherlosclerotic disease, peripheral vascular disorders, peritonitis, pernicious anemia, radiation therapy, Raynoud's disease, Senile Dementia, T-cell or FAB ALL, Telangiectasia, thromboangitis obliterans, varicose veins, vasculitis, venous diseases, venous thrombosis, Wilson's disease, xenograft rejection of any organ or tissue.

As used herein, “treatment” or any grammatical variation thereof (e.g., treat, treating, and treatment etc.), includes but is not limited to, alleviating a symptom of a disease or condition; and/or reducing, suppressing, inhibiting, lessening, ameliorating or affecting the progression, severity, and/or scope of a disease or condition. In some embodiments, a composition described herein may be used to assist in the treatment of a disease or condition along with an additional therapeutic agent or procedure. As used herein, an “effective amount” can refer to an amount that is capable of treating or ameliorating a disease or condition or otherwise capable of producing an intended therapeutic effect.

In some embodiments, the present disclosure also provides a pharmaceutical composition for treating disorder, disease or condition associated with impaired cell proliferation and/or differentiation of cells in a mammal, comprising an effective amount of any of the types of regulatory RNA preparations (e.g., total RNA preparations) according to the invention or any combination thereof, obtained from lymphoid cells of spleen, thymus, from peripheral blood lymphocytes, or from bone marrow of healthy donor or intact healthy donor subjected to activation of T-cellular component of the immune system, at the time when the cells manifest their stimulating (helper) or suppressing effect on somatic target cells of a particular cell type (e.g., histotype), and, optionally, a total RNA preparation(s) isolated from cord blood, umbilical cord and/or from the above mentioned somatic target cells, optionally together with a pharmaceutically acceptable carrier, diluent or excipient.

As used herein the term “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions. Pharmaceutical compositions can be prepared as described below. The active ingredients may be admixed or compounded with any conventional, pharmaceutically acceptable carrier or excipient. The compositions may be sterile.

A composition is said to be a “pharmaceutically acceptable carrier” if its administration can be tolerated by a recipient patient. Sterile phosphate-buffered saline is one example of a pharmaceutically acceptable carrier.

It will be understood by those skilled in the art that any mode of administration, vehicle or carrier conventionally employed and which is inert with respect to the active agent may be utilized for preparing and administering the pharmaceutical compositions of the present invention. Illustrative of such methods, vehicles and carriers are those described, for example, in Remington's Pharmaceutical Sciences, 4th ed. (1970), the disclosure of which is incorporated herein by reference. Those skilled in the art, having been exposed to the principles of the invention, will experience no difficulty in determining suitable and appropriate vehicles, excipients and carriers or in compounding the active ingredients therewith to form the pharmaceutical compositions of the invention.

The method of modulating proliferation and/or differentiation of mammalian cells for the treatment of diseases, disorders, or conditions, described herein, may be accomplished by administering to the subject a composition according to the invention with at least one of the routes selected from parenteral, subcutaneous, intramuscular, intravenous, intraarticular, intrabronchial, intraabdominal, intracapsular, intracartilaginous, intracavitary, intracelial, intracerebellar, intracerebroventricular, intracolic, intracervical, intragastric, intrahepatic, intramyocardial, intraosteal, intrapelvic, intrapericardiac, intraperitoneal, intrapleural, intraprostatic, intrapulmonary, intrarectal, intrarenal, intraretinal, intraspinal, intrasynovial, intrathoracic, intrauterine, vaginal, rectal, buccal, sublingual, intranasal (e.g., inhalation), and transdermal (e.g., topical), ophthalmic, ocular and aural routes of administration.

It should be noted that the intranasal route of administration in a number of cases is particularly preferred.

In an embodiment of the disclosure, the composition is formulated in accordance with routine procedures as a pharmaceutical composition adapted for intravenous, subcutaneous, intramuscular, intranasal, or topical administration to human or animal beings. Typically, compositions for administration by injection or infusion are solutions in sterile isotonic aqueous buffer. Where necessary (e.g., when the composition is presented in the lyophilized form), it may be also provided with a solubilizing agent. In certain cases, the RNA preparations for parenteral administration require sterilization. Sterility is ready accomplished by filtration through sterile filtration membranes, for example, prior to or following lyophilization and reconstitution. The parenteral route of administration includes known methods, e.g., injection or infusion by intravenous, intraperitoneal, intramuscular, intraarterial, intralesional or subcutaneous administration. Methods for preparing pharmaceutical compositions for parenteral, intranasal, and intralesional administration, formulations in the form of eye and ear drops are well known in the art and described in more detail in various sources, including, for example, Remington's Pharmaceutical Sciences and Introduction to Pharmaceutical Dosage Forms, 19th ed., Mack Pub. Co., Easton, Pa. (1995).

If the compositions of the invention are to be administered topically, the compositions can be formulated in the form of an ointment, cream, transdermal patch, lotion, gel, shampoo, spray, aerosol, solution, emulsion, or other form known to one of skill in the art (ibid).

Diverse experimental findings described in this application show convincingly that the total RNA preparations reproduce the functional properties of the original lymphoid cells and efficiently affect the morphogenetic processes, changing them in a necessary direction. Moreover, administration of preparations according to the invention can be used in place of a lymphoid cell transfer because the preparations have a potential to correct a dysfunction of somatic cells of any cell type (e.g., histotype) via natural regulatory mechanisms.

Variability of functional manifestations of total RNA under the influence of various factors acting on the cell from which it was obtained, suggests that its use in medical practice can find a very wide application.

As our long-term studies have shown, the morphogenetic function of lymphoid cells has its features and regularities. In particular, organ specificity is predominantly characteristic of the morphogenetic function of lymphoid cells. This means that lymphoid cells adoptively transferred to a recipient inevitably affect cell proliferation in the organ that is the same as the donor organ exposed to a damaging factor (e.g., surgery) or any other factor activating T-cell immunity. It should be noted that proliferative activity of the recipient's lymphoid cells always changes in the direction that corresponds to the signal transferred.

Two phases are distinguishable in the effect of regeneration-activated lymphoid cells, as well as in the regeneration process itself. Lymphocytes that are capable of stimulating cell proliferation in the target organ are the first to act, while lymphocytes capable of suppressing cell division in the target organ appear later, at peak proliferation. The latter do not prevent completion of the mitotic cycle in the cells that have already started division, but prevent new cells from entering the mitotic cycle. By this means, the lymphocytes facilitate completion of the cell proliferation wave and stop the restorative process, thereby preventing hyper regeneration. Thus, lymphocytes ensure both the start and completion of the regeneration process.

As we have shown, different T-cell populations, which possess T-helper or T-suppressor properties, are responsible for the stimulatory and inhibitory functions. The effect of T suppressors is organ specific to a lesser extent than that of T stimulators. Data on the lymphoid regulation of morphogenetic processes have been summarized in several monographs [Babaeva A. G. Immune Mechanisms Controlling Regeneration. Moscow, 1972, 150 pp.; Babaeva A. G. Regeneration and the System of immunogenesis. Moscow, 1985, 256 pp.; Babaeva A. G., Gevorkyan N. M., Zotikov E. A. The Role of Lymphocytes in Switching over Tissue Development Programs. Moscow: Ross. Akad. Med. Nauk, 2009, 108 pp.]. It should be emphasized here that a modus of enhanced cell proliferation, which is characteristic of regeneration, is induced in the recipient by activated lymphocytes possessing a stimulatory potential. Yet all of the above processes occur in a syngeneic system only. In an allogeneic system, this potent natural regulator of proliferative and restorative processes could not be used because of antigenicity-related limitations associated with immunological incompatibility and, consequently, did not find application in medicine.

At the same time, advances in surgery and transplantation required the incompatibility problem to be solved, providing great impetus to searching for solutions. The problem became especially pressing in hematology and transfusion medicine as demand for bone marrow transplants and repetitive blood transfusions increased, while substantial obstacles persisted to hinder their unrestricted use.

Blood, blood cell, and other blood component transfusions are still the most common and in-demand invasive interventions in medicine Millions of lives were saved by transfusions, and several generations of doctors and researchers worked to make this technically simple procedure safe for the patients.

In spite of the great achievements, certain safety concerns are still associated with blood transfusions, being determined by both the properties of the tissue to be transferred (the blood) and the specific of the recipient (human) as a biological species. An immune conflict arising in the case of antigenic incompatibility of the donor and recipient is life threatening. The total history of blood transfusion is associated with the need to select a compatible donor, which is crucial for the procedure. The human blood is the most immunogenic tissue for all mammals, especially humans. Blood cells are extremely abundant in antigens, i.e., erythrocytes carry the AB0 antigens and several variants of the Rhesus factor, M and N rare erythrocyte antigens, and several other antigens; up to 40 specific antigens have been found on platelets; and lymphocytes carry more than 100 antigens of the HLA (human leukocyte antigen) system, which is a continuously increasing group of antigens determined by the major histocompatibility complex (MHC) in human. As a result, finding a compatible donor among unrelated people is highly problematic (which is determined by the number of combinations of different antigens) and usually requires a screening of several thousands of donor blood samples.

Typed donor blood banks have been organized to solve the problem. Yet only donor selection by the AB0 and Rhesus system antigens can be considered a generally solved problem now. As for the rare erythrocyte antigens, it cannot be excluded that a recipient is sensitized to them. Rare-antigen incompatibility does not manifest itself after the first transfusion, while a repeated transfusion or, for instance, same-specificity incompatible pregnancy lead to an immune conflict, isoimmune anemia, etc. The same nature is possible for other cytopenias, such as thrombocytopenia, neutropenia, lymphocytopenia, and immune platelet refractoriness.

To prevent these complications, not only a donor is thoroughly selected, but transfusions of particular blood components (erythrocytes, platelets, or leukocytes) are used in place of whole blood transfusions, thus reducing the risk of additional sensitization. In the cases where multiple blood transfusions are certainly necessary, bone marrow is transplanted as a long-lived source of necessary blood, while blood cells have a limited life after transfusion, and, on the other hand, quicker regeneration of the recipient's hematopoietic tissue is achieved using various agents to stimulate the regulatory systems of normal and reparative hematopoiesis.

The lymphoid regulation occupies a special place among systems regulating the restorative processes.

The immunogenesis system generally controls proliferation of the majority of cells in the body, ensuring the constant cell population size and the anatomical integrity of organs and tissues in normal conditions, as already mentioned. The morphogenetic function was theoretically grounded and experimentally demonstrated in the late 1960s in a model of adoptive lymphocyte transfer, wherein lymphocytes transferred regeneration information from partially hepatectomized animals to intact syngeneic recipients [Babaeva A. G. Immune responses in normal and regenerative growth. In: Regeneration and Cell Division. Moscow, 1968, pp. 11-16]. The regular character of the phenomenon was verified in experiments with several organs (the kidney, intestine, hematopoietic tissue, lung, and skin) by both the authors of the primary publication and other researchers. One of the most important features of this regulatory system is that it acts in a targeted manner, displaying a predominant (thought, not absolute) organ and tissue specificity. This means that the lymphoid regulation exerts its morphogenetic effect mostly on the organs and tissues that have been affected by a pathogenic factor or surgery. The immunogenesis system has the possibilities of an ideal natural regulatory system. Its cells ad their biologically active products (lymphokines) initiate cell proliferation (T cells with T-helper properties (T effectors)), stop cell proliferation (T cells with T-suppressor properties (T regulators)), and eliminate altered cells, such as cells changed by pathogenic factors (T killers). The factors produced by lymphoid cells facilitate the lymphocyte interactions with each other and with other lymphoid cells and cells of target organs. When an extreme situation arises in natural conditions (e.g., regeneration starts in response to damage), the function of the proper populations increases to allow a successful completion of regeneration. In a situation that does not take place in nature (e.g., organ transplantation or adoptive transfer of foreign cells), the immunogenesis system protects the body from alien material.

Allotransplantation of tissues, including bone marrow, and blood transfusion are among the situations that do not occur in nature, and transplants are rejected by the immune system, which recognizes alien material by numerous combinations of antigenic differences (interspecific and inter-individual).

To overcome the problem, not only compatible donors and recipients are selected, but immunosuppression is usually induced in the recipient to prevent the recipient's immune system from rejecting foreign tissue. However, numerous adverse effects are well known for immunosuppression.

Seeking a solution to the problem, we developed a more efficient method of transplantation or adoptive transfer by replacing cells with the cell components that preserve functional activity of the original cells to a certain extent, but lack their antigenic properties. Total RNA isolated from cells proved to be such a component. RNA has virtually no individual and species-specific antigenic determinants according to published data. For instance, xenogeneic yeast RNA is well tolerated by mammals, including humans, and is used in medicine to treat several disorders, such as eye diseases, Sjogren's disease, degenerative diseases of the neuromuscular system, hereditary myopathies, neuroinfection sequelae, and spinal amyotrophy, both as oral formulations and intramuscular injections [Shabanova M. E., Kazaniev V. V., Baurina M. M., Krasnoshtanova A. A., Krylov I. A. A method for enhancing proliferative activity of the bone marrow. In: Neuroimmunopatology (Abstr. Fourth Russian Conference). Patogenez, 2006, no. 1, p. 71]. Shabanova et al. (2006) have shown in experiments with rats that bone marrow taken from rats injected with yeast RNA produces twice as many colonies (of the erythrocytic, granulocytic, and megakaryocytic lineages) in the spleen of irradiated recipient rats. Other studies of the RNA effect on the body focus mostly on the improvements in functional parameters and immune functions, including the effects on reactivity, resistance to infection, immunity, and functional activity of macrophages and lymphoid cells. Although the favorable effects of RNA are commonly attributed to its trophic function, its possibilities are actually far greater because there is evidence that, in experiments with adoptive transfer, RNA is capable of transmitting the morphological and functional specifics that have arisen in the lymphoid cells used as an RNA source in response to environmental changes or damaging factors.

We focused on the questions as to whether a total RNA preparation derived from lymphoid cells is capable of the specific regulatory function inherent in the cells, regulating proliferation and functionally substituting the cells, and whether its effect preserves the predominant organ specificity, which is characteristic of the lymphocyte effect, for instance, in animals with induced anemia. To study this, we tested total RNA preparations for effect on erythropoiesis and hematopoiesis in total in regulatory RNA recipients. The questions were answered using animals with induced anemia and other in vitro and in vivo models for studying erythropoiesis and hematopoiesis.

For the purpose, we used several models suitable for detecting the regulatory properties of lymphoid cells both in cell culture in vitro and in vivo. Our earlier experiments in an animal model with adoptive transfer of lymphocytes showed that the regeneration process is always accompanied by phasic changes in the morphogenetic properties of lymphocytes; i.e., the capability of stimulating cell proliferation gives place to the capability of suppressing it to prevent an excessive growth of the regenerating organ. Hence, one of the primary objectives was to study whether similar phasic changes occur in the functional properties of RNA, for instance, in the case of blood regeneration, that is, whether the total RNA preparation is similar to the original lymphoid cells in inducing the modus of enhanced cell proliferation and differentiation.

The blood was chosen as a primary subject to study the functional properties of total RNA preparations derived from lymphoid cells. With the example of hematopoiesis, the effect was examined for total RNA preparations obtained in normal conditions, that is, from intact lymphoid cells (preparation RNA-1) and in the case of blood regeneration in the phases when lymphoid cells exert their stimulating (preparation RNA-2) or suppressing (preparation RNA-3) effect on hematopoiesis. Activity of total RNA preparations was additionally assayed at different functional states of the recipient's hematopoietic tissue, including (a) physiological hematopoiesis, (b) increased hematopoiesis, and (c) acute or chronic inhibition of hematopoiesis.

The total set of our experiments without an exception showed clearly that a total RNA preparation derived from lymphoid cells possesses those of the above morphogenetic properties that are inherent in the original lymphoid cells.

Some of the preferred embodiments are described below using specific examples, with reference to the accompanying figures, in order to facilitate the understanding and practical implementation of the present invention.

### EXAMPLES

### Materials and Methods

Experiments were performed on young mature outbred white rats of both sexes and cultured rat bone marrow erythroblastic islets (EIs). Mice of the C57BL/RsJYLeprdb/+ strain with manifest type 2 diabetes mellitus (the Svetlye Gory nursery) were used in some experiments. The animal were handled according to the European Convention for the Protection of Vertebrate Animals Used for Experimental and Other Scientific Purposes (Strasbourg, 1986) and the international regulations stated in the Good Laboratory Practice for Nonclinical Laboratory Studies of 4 Mar., 2002.

The rats used in the study with a body weight of 180-220 g were kept in standard plastic cages and fed on the standard vivarium ration with unlimited access to water at an air temperature of 18-25° C.

Activation of lymphoid cells for obtaining total RNA preparations was performed by bleeding, with a blood loss of 2% of the body weight. For obtaining spleen lymphoid cells with stimulatory and inhibitory activities, the rats were euthanized by ether anesthesia 17 or 96 h after the acute blood loss, respectively. These intervals of time after blood loss (or “donor intervals”) were selected exclusively for convenience of experiments, because the effective periods of the stimulatory and inhibitory activities were rather long (between about 15 min and 48 h after blood loss for the stimulatory activity and between about 48 and 96 h or more for the inhibitory one). They have been shown to depend on the species and age of the animal, the severity of surgical or damaging intervention, and the target organ or tissue subjected to the intervention [Babaeva A. G. Regeneration and the System of Immunogenesis. Moscow, 1985, 256 pp.]. These periods are almost the same for rats and mice.

Total RNA was isolated from spleen and thymic lymphoid cells, unfractionated bone marrow of young rats of both sexes weighing 80-130 g, and human peripheral blood lymphocytes (donor blood); in addition, preparations of total RNA were the same way obtained from human and rat cord blood, umbilical cord, and placenta.

Using the procedure of determining the amount of RNA and DNA in the preparations obtained by us [Trudolyubova M. G. Quantitative assay of RNA and DNA in subcellular fractions of animal cells by a modified Schmidt-Thannhauser method. In: Modern Methods in Biochemistry. Ed. by Orekhovich V. N. Moscow: Meditsina, 1977, pp. 313-316], it was shown that DNA content in them is zero. Likewise, they do not contain protein that has been shown by the micro biuret method of determining protein.

Total RNA was isolated by the standard method using the trizol reagent and thiocyanate-phenol-chloroform extraction [Chomczynski P. A reagent for the single-step simultaneous isolation of RNA, DNA and proteins from cell and tissue samples. BioTechniques, 1993, vol. 15, pp. 532-537]. A preservative solution (e.g., fixative IntactRNA, Evrogen, Cat. # BCO31, RNALater, or other preservative or combination thereof) can be used in order to increase the stability of RNA molecules by separating them from their natural microenvironment (e.g., separated from host cells). In some cases, cells isolated from different organs were not mixed, and their functional properties were determined separately.

Total RNA preparations were derived from different lymphoid organs of intact animals, as well as from lymphoid organs of anematized animals at different time points after bleeding. It should be noted that the total RNA preparations from the lymphoid cells at the stages when the cells exhibited the stimulatory and inhibitory effects also had, respectively, the stimulatory and inhibitory effects on the cells with the same or other histotypes. Thus, three basically different types of RNA preparations were obtained: RNA-1 (from intact animals, i.e. from non-activated lymphoid cells), RNA-2 possessing a stimulatory activity, and RNA-3 possessing an inhibitory activity. These activities were observed both in a culture model (in vitro) and in experiments on animals (in vivo). It should also be noted that the preparation of total RNA isolated from lymphoid cells at the step when helper or suppressor activity is manifested, is a preparation having correspondingly helper or suppressor cell activity towards the cells of the same and/or different histotype both in a recipient's body and respective cultured target cells in vitro.

Using electrophoresis in 1.5% agarose, it was found in particular that the regulatory preparations of total RNA isolated from lymphoid cells of one or another lymphoid organ and in this or that phase of the process of morphogenesis (regeneration) may comprise RNA molecules of about 50 to about 50,000 or more nucleotides, e.g., about 50 to about 400 or more nucleotides, about 50 to about 3,000 or more nucleotides, about 50 to about 10,000 or more nucleotides. Thus, the claimed regulatory properties may be associated with RNA molecules having a molecular weight of from about 15 kDa to about 18,000 kDa or higher, or the nucleotide numbering from about 50 to about 50,000 or more nucleotides, for example about 136,000 nucleotides (in particular, for example, 135,639 nucleotides), and even higher macromolecular RNA samples.

Preparations according to the invention for in vitro studies were used at a dose of 2 μg/ml culture medium or 4 μg/ml culture medium. In in vivo experiments, the preparations were administered parenterally at doses from 5 to 30 μg/100 g body weight. Note that these doses are tens to hundreds of times lower than the doses used by other authors [Shabanova M. E., Kazaniev V. V., Baurina M. M., Krasnoshtanova A. A., Krylov I. A. A method for enhancing proliferative activity of the bone marrow. In: Neuroimmunopathology (Abstr. Fourth Russian Conference). Patogenez, 2006, no. 1, p. 71; Patent RU 2,238,756 (2003)].

The erythropoietic activity of the preparations were determined in a culture of erythroblastic islets (EIs) of the rat bone marrow (BM) using an in vitro model of physiologically normal erythropoiesis [Tishevskaya N. V., Zakharov Yu. M., Tishevskoy I. A. Effect of erythropoietin at different concentrations on cultured erythroblastic islets. Ross. Fiziol. Zh. im. I. M. Sechenova, 1998, vol. 84, no. 12, pp. 1412-1419]. When developing the method for maintaining erythropoiesis in cultured BM EIs at the physiologically normal level, the authors of this model showed that the normal hematopoiesis rate in the culture was reached at an erythropoietin concentration of 0.5 IU/ml. This suggests that the qualitative compositions of EIs in intact animals in vivo and in cultured BM EIs in the presence of 0.5 IU/ml erythropoietin are the same.

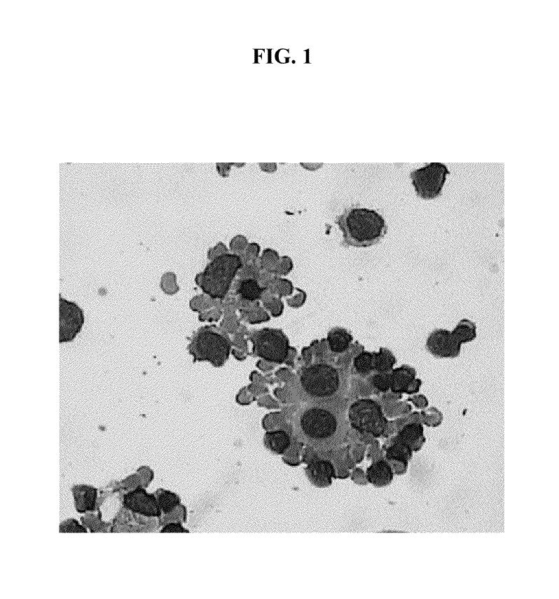

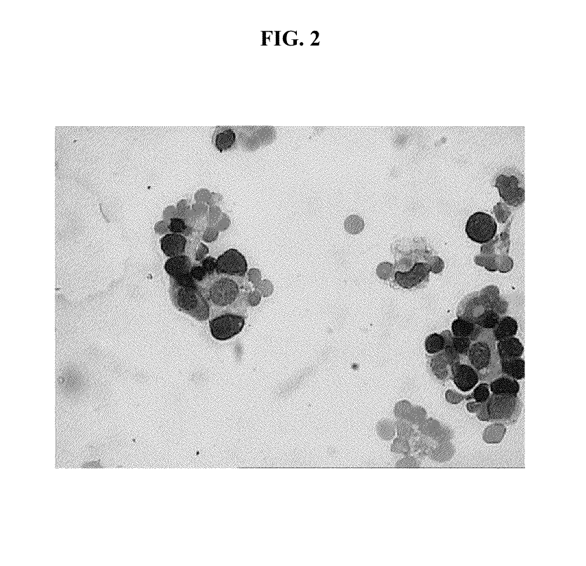

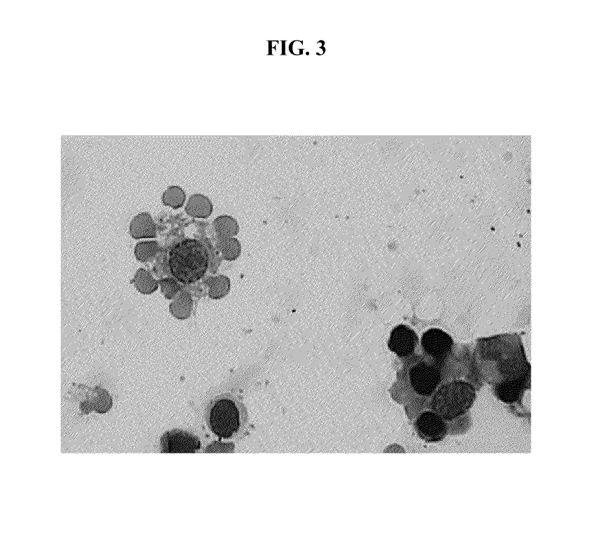

In the EI cultures, we determined the absolute number of EIs per 1 cm2 of the surface of the culture dish and their distribution among classes of maturation according to the classification suggested by Zakharov et al. [Zakharov Yu. M., Melnikov I. Yu., Rassokhin A. G. Classification of bone marrow erythroblastic islets taking into account their cell composition. Arkh. Anat. Gistol. Embriol., 1990, no. 5, pp. 38-42], taking into account the number and differentiation stages of erythroid cells in the “crown” surrounding the central macrophage. Zakharov et al. (ibid) distinguish the following EI maturation classes: (1) class 1 EIs whose “crown” is formed by proerythroblasts, erythroblasts, and basophilic normoblasts (2-8 cells); (2) class 2 EIs whose “crown” contains basophilic and early polychromatophilic normoblasts (9-16 cells); (3) class 3 EIs whose “crown” contains middle and late polychromatophilic normoblasts, oxyphilic normoblasts, and reticulocytes (17-32 cells); (4) involuting EIs (Inv. EIs) whose “crown” contains late polychromatophilic and oxyphilic normoblasts and reticulocytes (no fewer than 16 nucleated cells); and (5) reconstructing EIs (Rec. EIs), which differ from Inv. EIs in that juvenile cells capable of proliferation, including proerythroblasts, erythroblasts, and/or basophilic normoblasts, are attached to their “crown” (as in class 1 and class 2 EIs) (FIGS. 1-3).

The intensities of the formation of EIs and the development of their erythroid “crown” were estimated using the following calculated indices [Vorgova L. V., Zakharov Yu. M. Changes in bone marrow erythroblastic islets in animals upon combined thermal and muscular loads. Fiziol. Zh. SSSR im. I. M. Sechenova, 1990, vol. 76, no. 2, pp. 200-206]:

(1) The “index of CFU-e involvement in differentiation” (CFU-e is a colony-forming unit of the erythroid lineage) calculated as

EI1+EIrec,

wherein EI1 and EIrec are the numbers of class 1 EIs and Rec. EIs, respectively.

(2) The “index of erythroid cell maturation” in EIs calculated as the ratio of the total number of EIs whose “crown” contains mature cells to the total number of EIs of proliferative classes:

(EI3+EIinv)/(EI1+EI2+EIrec),

wherein EI3, EIinv, and EI2 are the numbers of class 3 EIs, Inv. EIs, and class 2 EIs, respectively.

(3) The “index of macrophage reinvolvement” in erythropoiesis calculated as

EIrec/EIinv.

A significant increase in the numbers of class 1, class 2, and Rec. EIs accompanied by a significant decrease in the number of Inv. EIs was regarded as an indicator of erythropoiesis stimulation.

Conversely, a decrease in the numbers of class 1, class 2, and Rec. EIs accompanied by an increase in the number of Inv. EIs was regarded as an indicator of erythropoiesis inhibition.

The preparations to be tested were added to the culture medium in Petri dishes containing equal numbers of EIs (1500 EIs in 3 ml of medium per dish).

Several series of experiments were carried out to study the effects of the RNA-1, RNA-2, and RNA-3 preparations on erythropoiesis.

Initially, we derived the RNA preparations from spleen lymphoid cells, because the morphogenetic function of lymphocytes was first found in experiments on adoptive transfer of spleen lymphocytes [Babaeva A. G. Immune Mechanisms Controlling Regeneration. Moscow, 1972, 150 pp.; Babaeva A. G. Regeneration and the System of Immunogenesis. Moscow, 1985, 256 pp.]. According to the objectives of the study, we initially obtained three RNA preparations: RNAs-1 from spleen lymphoid cells of intact rats; RNAs-2 from rat spleen lymphoid cells isolated 17 h after an acute blood loss (2% of the body weight), which had a stimulatory effect on erythropoiesis (the preparation obtained at this time point has been described in detail in our earlier studies); and RNAs-3 from rat spleen lymphoid cells isolated 96 h after the acute blood loss, which had an inhibitory effect on erythropoiesis. Then, we used the same method to obtain, at the same stages of regeneration, similar preparations of total RNA from the bone marrow (BM) (RNAbm-2 and RNAbm-3) and thymus (RNAt-2 and RNAt-3) of anematized rats, as well as from these organs of intact rats (RNAbm-1 and RNAt-1, respectively). The same procedure can be used to obtain total RNA preparations from lymph nodes of animals in the intact state and at different stages of regeneration, as well as from any cell population of the body, including stem cells. Our data show that the total RNA preparations introduced into the recipient's body “reproduce” there the function of the cells from which they have been derived (or facilitate the same function of the corresponding recipient's cells).

In the first series of experiments, we studied the effects of the preparations according to the invention on physiologically normal erythropoiesis in vitro, i.e., on the erythropoiesis rate in BM EIs obtained from intact rats.

Rat BM EIs were cultured in a multigas flow incubator (SANYO, Japan) with an auto-decontamination system and an automated control of CO₂ supply. The relative humidity of the atmosphere in the incubator was maintained at 95%. The set temperature was maintained to an accuracy of ±0.15° C. at 37° C., with a temperature gradient in the incubation chamber varying within ±0.3° C.

All manipulations used in preparing culture medium components, isolating and suspending EIs, and filling Petri dishes containing adhered EIs with the prepared culture medium were performed in an SShL-0.5/130 laminar flow hood (ZAO Asepticheskie Meditsinskie Sistemy, Miass, Russia) in a vertical descending low-turbulence air flow. The degree of purification of the supplied air from suspended particles larger than 0.5 μm was 99.95%.

The culture viability was monitored by means of phase contrast microscopic examination using a Biolam P-1 inverted microscope with a 10×0.22 lens and an AU-12 binocular adjustment with a magnification of 1.5. For estimating the cell composition of the cultures, they were fixed, stained, and examined under a TS-136 laboratory binocular microscope (Tenso, Germany).

Erythroblastic islets were cultured in separate sterile plastic Petri dishes 35 mm in diameter (Corning-Costar). RPMI-1640 was used as the basic constituent of the EI culture medium. It was supplemented with 146 mg/l glutamine and 7.5% sodium bicarbonate added to a final concentration of 26.7 mg/l [Goldberg E. D., Dyhai A. M., Shakhov V. P. Tissue Culture Methods in Hematology. Tomsk, 1992, 272 pp.]. 2-Mercaptoethanol was used as an antioxidant and a reducer of sulfhydryl groups. In order to provide the developing erythroid cells with growth factors, enzymes, other proteins, and lipids, the culture medium was supplemented with fetal calf serum tested for cytotoxicity and the absence of mycoplasma (the high-quality serum jointly produced by German and French companies that was demonstrated to ensure the best culture growth when tested in PanEco Company). Shortly before the experiment, a mixture of antibiotics toxic for Gram-positive and Gram-negative bacteria, namely potassium salt of benzylpenicillin (C16H17N2O4SK, MM=372.5 Da) and streptomycin (C21H39N7O12×1,5H2SO4, MM=728.7 Da), was diluted with RPMI-1640 and added to the culture mixture to a concentration of 0.05 mg/ml (synthesis of nucleic acids in cells is inhibited by concentrations higher than 1 mg/ml). For stimulating the functional activity of central macrophages of EIs and forming a “store” of cytokines around the developing cells, the culture medium was supplemented with heparin, which increases the adhesive capacity of cultured cells and activates the proliferation of erythroid, myeloid, and monocyte cells [Luikart S. D., Sackrison J. L., Manglia C. A. Bone marrow matrix modulation of HL-60 phenotype. Blood, 1987, vol. 70, pp. 1119-1126; Luikart S. D., Manglia L. T., Furch J. B. A heparan sulfate fraction of bone marrow induces maturation of HL60 cells in vitro. Cancer Res., 1990, vol. 50, pp. 3781-3791; Yushkov B. G., Popov G. K., Severin M. V., Yastrebov A. P. Glycosaminoglycans and Erythropoiesis. Yekaterinburg: Ural. Otd. Ross. Akad. Nauk, 1994, 127 pp.].

The complete composition of the culture medium used in the experiment was the following (per 100 ml):

RPMI-1640 medium, 62 ml

Fetal calf serum, 30 ml

Heparin, 1.3 ml (6500 U)

Benzylpenicillin, potassium salt, 1 ml (5000 U)

Streptomycin, 1 ml (5 mg)

2-Mercaptoethanol, 1 ml of stock solution

L-glutamine, 1 ml (14.6 mg)

Sodium bicarbonate, 2.7 ml of 7.5% solution

The components of the culture medium were mixed under sterile conditions shortly before the experiment, and the prepared medium was filtered through an acetate filter with 0.22-μm pores.

Recormon, a recombinant human erythropoietin (Boehringer Mannheim GmbH, Germany), was added to the culture medium at a dose of 0.5 IU/ml to stimulate the growth of cultured EIs.

Erythroblastic islets were isolated from the BM of the femoral bones using the technique suggested by Zakharov et al. [Zakharov Yu. M., Melnikov I. Yu., Rassokhin A. G. Study of erythropoiesis by a modified method of isolation of bone marrow erythroblastic islets. Gematol. Transfuziol., 1984, vol. 29, no. 4, pp. 52-54], which is a modification of the technique developed by Charpentier and Prenant in 1975 [Charpentier Y., Prenant M. Isolement de l′ilots erythroblastique. Etude en microscopie optique et electronique a balayage. Nouv. Rev. Franc. Hemat., 1975, vol. 15, pp. 119-140]. The bone marrow was obtained by washing the femoral bone cavity with 1.5 ml of preparation medium, which had the same composition as the culture medium except that it did not contain 2-mercaptoethanol and erythropoietin. The resultant suspension of EIs and single BM cells was placed onto the surface of Petri dishes by means of a dispenser pipette.

In order to separate EIs from the suspension of BM cells, the Petri dishes were placed into a gas-flow incubator for 30 min at a temperature of 37° C., relative humidity of 95%, and CO₂ content of 4.5%. After the incubation, nonadherent elements of BM were washed off from the EI monolayer with the RPMI-1640 medium by means of a hypodermic syringe. After that, the Petri dishes were filled with the culture medium, the tested preparations were added using a microdispenser pipette, and the dishes were placed into a gas-flow constant-temperature cabinet under the conditions indicated above. The cultivation was carried out for 24 h.

Each of the three preparations, RNAs-1, RNAs-2, and RNAs-3, was added to the Petri dishes completely prepared for cultivation at a dose of 2 or 4 μg/ml culture medium shortly before the dishes were put into the incubator. Each preparation was tested on 30 EI cultures. Control BM EIs obtained from intact rats were cultured without addition of the preparations simultaneously with the experimental cultures under the same conditions (10 control cultures for each preparation). The same number of cultures was used for estimating the background state shortly before cultivation. A total of 40 male outbred white rats aged 4-5 months with a body weight of 140-160 g were used.

In the second and third experimental series, the effects of the RNAs-1 and RNAs-2 preparations were tested on the cultures of BM EIs obtained from rats in which erythropoiesis was inhibited by experimental polycythemia (model (I) of post-transfusion polycythemia).

Model (I) of Post-Transfusion Polycythemia.

To obtain a model of inhibited erythropoiesis, we took blood from the superior vena cava of donor rats (weighing 250-300 g) and centrifuged it three times in 0.9% NaCl to obtain an 80% erythrocyte suspension. This suspension was injected once, intraperitoneally to recipient rats (weighing 90-100 g) at a dose of 7 ml/100 g body weight. BM EIs were isolated from the femoral bones of polycythemic rats on the fifth day after the transfusion of erythrocyte suspension, when the amount of reticulocytes in the blood of BM donors was decreased by half.

Rats with experimental polycythemia served both as donors of EIs for studying their response to the RNA preparations in vitro and as recipients of these preparations in in vivo experiments. Both variants of this model were used to evaluate the stimulatory effect of RNA from spleen lymphoid cells on the development of BM erythroid cells under the conditions of initially suppressed erythropoiesis; e.g., to stimulate erythropoiesis, the RNA preparations had to first overcome the suppression of erythropoiesis induced by polycythemia.

On the fifth day after the transfusion of the erythrocyte concentrate, we determined the hematocrit, erythrocyte count, hemoglobin concentration, and reticulocyte count in the peripheral blood of the rats. The animals in which the hematocrit was higher than 60%, the erythrocyte count was no lower than 9×10¹²/l, and the reticulocyte count was decreased at least by half compared to the initial level were used in the experiments. On the same day (day 5 after transfusion), five rats were intravenously injected with RNAs-1 and five rats, with RNAs-2 (from intact donors and anematized donors subjected to a blood loss of 2% of body weight, respectively) at a dose of 15 μg/100 g body weight (groups RNAs-1 and RNAs-2). The control group consisted of five rats with post-transfusion polycythemia who were euthanized on day 5 after the erythrocyte concentrate transfusion to determine the background level of polycythemia. Earlier, we and other researchers demonstrated that severe suppression of erythroid hematopoiesis in rats, which began immediately upon injection of an 80% homologous erythrocyte suspension, peaked by day 5 and was retained until day 10 after transfusion [Melnikov I. Yu. Study of bone marrow erythroblastic islets at different functional states of erythropoiesis. Cand. Sci. (Med.) Dissertation. Chelyabinsk, 1987, 200 pp.; Rassokhin A. G., Kruglov D. G., Zakharov Yu. M. The state of erythropoiesis and functional characteristics of bone marrow erythroblastic islets upon stimulation and inhibition of erythropoiesis. Vestn. Ross. Akad. Med. Nauk, 2000, no. 2, pp. 9-14; Tishevskaya N. V. The time course of glycosaminoglycan composition at different states of erythropoiesis in erythroblastic islets. Cand. Sci. (Med.) Dissertation. Chelyabinsk, 1995, 112 pp.; Moiseeva O. I. Physiological Mechanisms of Erythropoiesis Control. Leningrad: Nauka, 1985, 183 pp.; Filimonov V. I. Mechanisms of erythropoiesis inhibition. Patol. Fiziol. Eksp. Ter., 1972, vol. 16, no. 5, pp. 33-37; Gitelzon II., Sidko S. F. Cell population mechanisms of erythropoiesis suppression in polycythemia. Tsitologiya, 1977, vol. 19, no. 6, pp. 632-638; Rassokhin A. G. Effect of erythropoietin on erythropoiesis in bone marrow erythroblastic islets in polycythemia. In: Proc. II Congr. of Physiologists of the Urals, 1990, pp. 29-30]. In this period, the total number of EIs and the proportion of EIs of proliferating classes in the BM were drastically decreased, super-islets with more than 64 erythroid cells in the “crown” appeared, the indices of CFU-e involvement in differentiation and macrophage reinvolvement were decreased, and the number of resident macrophages was increased; simultaneously, the number of reticulocytes in the peripheral blood was two to three times decreased. After that, the peripheral blood parameters of the experimental and control animals was studied by the standard methods daily until day 10 of the experiment. On day 10, the experiment was terminated, and the BM was isolated in order to estimate erythropoiesis in BM EIs. The said classification suggested by Zakharov et al. [Zakharov Yu. M., Melnikov I. Yu., Rassokhin A. G. Classification of bone marrow erythroblastic islets taking into account their cell composition. Arkh. Anat. Gistol. Embriol., 1990, no. 5, pp. 38-42] was used for the qualitative estimation of erythropoiesis.

The effect of RNAs-3 on erythropoiesis in cultured BM EIs was studied in two models: Model (II) of physiologically normal erythropoiesis (without stimulation) and Model (III) of compensatory erythropoiesis stimulated with an acute blood loss (2% of the body weight). A total of 30 cultures (5 cultures in each group) were examined.

In order to clarify whether the preparation stimulation erythropoiesis was also effective in vivo under the conditions of pathology and estimate its effect, we used two experimental models: benzene-induced hypoplastic anemia and sub-lethal irradiation of rats.

Model (IV) of Benzene-Induced Chronic Hypoplastic Anemia.

Anemia was induced in rats weighing 130-250 g with normal parameters of peripheral blood by three subcutaneous injections of a mixture of equal volumes of benzene and vegetable oil, the dose of benzene being 0.05 ml/100 g body weight. The injections were made at seven-day intervals.

The state of the hematopoietic system as reflected by peripheral blood parameters in the rats with benzene-induced anemia was monitored by weekly determining the erythrocyte, reticulocyte, leukocyte, and platelet counts. Four weeks after the last benzene injection, the leukocyte and platelet counts were significantly decreased by a factor of 4.5, and the reticulocyte count was decreased by a factor of 7.

This was the hematopoietic background when, four weeks after the last benzene injection, we started the administration of the tested RNA-1 and RNA-2 preparations, which were injected three times at ten-day intervals. Control animals were injected with 0.9% NaCl on the same days.

We also performed an additional experiment, where, four weeks after the last benzene injection, we started the administration of the RNA preparation derived from the bone marrow of anematized rats at the stage of hematopoiesis stimulation (in our case, 17 h after a blood loss of 2% of the body weight) to animals with manifest benzene-induced anemia. The preparation was injected at a dose of 15 μg/100 g body weight three times at ten-day intervals. Control animals were injected with 0.9% NaCl on the same days.

Thus, each experimental animal received a total dose of an RNA preparation of 45 μg/100 g body weight. The peripheral blood parameters listed above were estimated every ten days.

Model (V) of Sub-Lethal Irradiation.

Fifteen female outbred rats weighing 140-170 g were subjected to single-exposure γ-irradiation at a sub-lethal dose of 6 Gy. Then, the animals were subdivided into three equal groups, one of which became a control group and the other two, experimental groups. Two hours after irradiation, the animals of one experimental group were intravenously injected with the RNA preparation from intact bone marrow (RNAbm-1) at a dose of 30 μg/100 g body weight, and the animals of the other experimental group, with the same dose of the RNA preparation from bone marrow stimulated with a blood loss of 2% of the body weight (RNAbm-2). As in all other experiments with stimulation, the bone marrow for obtaining RNA was isolated 17 h after the blood loss. Seven days after irradiation, the animals of each experimental group were injected with the same RNA preparation (RNAbm-1 or RNAbm-2) at a dose of 20 μg/100 g body weight. On day 14 after irradiation, the animals received the third injection of the RNA preparations at a dose of 30 μg/100 g body weight, but this time they were injected with preparations derived from lymphoid cells of the thymus, rather than bone marrow, of intact and anematized rats (RNAt-1 and RNAt-2, respectively) as described above. Control animals were intravenously injected with the same volumes of 0.9% NaCl on the same days.

On days 3, 5, 7, 10, 15, 20, 25, 31, and 40 of the experiment, the blood cell (reticulocyte, erythrocyte, leukocyte, and platelet) counts in the blood of the experimental and control animals were determined by the standard methods. On day 45, the animals were withdrawn from the experiment to estimate the state of their bone marrow hematopoiesis. The cell composition of the bone marrow was determined by myelography using the standard method [Filimonov V. I. Mechanisms of erythropoiesis inhibition. Patol. Fiziol. Eksp. Ter., 1972, vol. 16, no. 5, pp. 33-37]. To analyze the characteristics of erythropoiesis regeneration, we determined the total number of EIs (expressed in thousands of EIs per femoral bone) in a bone marrow suspension and, after the EI adhesion to Petri dishes, the EI distribution among maturation classes according to Zakharov et al. [Zakharov Yu. M., Melnikov I. Yu., Rassokhin A. G. Classification of bone marrow erythroblastic islets taking into account their cell composition. Arkh. Anat. Gistol. Embriol., 1990, no. 5, pp. 38-42].

The results were treated by the standard descriptive statistical methods; the mean values, errors of the mean, confidence intervals, and standard deviations were calculated. The groups were compared using the nonparametric Kolmogorov-Smirnov, Mann-Whitney, and Kruskal-Wallis tests. The differences were considered significant at a probability of type I error <0.05.

Lymphocytes were isolated from heparinized blood (10-15 units of heparin per 1 ml of blood) 1:1 diluted with physiological saline and applied onto 2 ml of ficoll-verographin density gradient with a specific density of 1.077 g/ml. Centrifugation was performed at 1500 rpm (400 g) for 25 min using an OPN-3 centrifuge. The lymphocyte “ring” that formed in the density gradient was collected into separate centrifugal test tubes. The cells were washed with 10 ml of physiological saline using the following centrifugation profile: one centrifugation at 400 g for 10 min and one centrifugation at 350 g for 6-7 min (to remove platelets).

Model (VI) of Alloxan-Induced Type 1 Diabetes Mellitus (DM).

We used the alloxan-induced diabetes mellitus model. Alloxan has a triketonic structure similar to that of glucose. It is selectively bound by glucose transporter 2 (GLUT-2) and is transported into β-endocrinocytes of Langerhans islets of the pancreas. Oxidation of alloxan in the β-endocrinocyte cytoplasm yields free-radical metabolites, which cause massive necrosis of Langerhans islets leading to absolute insulin deficiency.

Modeling Type 1 DM:

1. Intact rats weighing 180-220 g were subcutaneously injected with Freund's complete adjuvant at a single dose of 0.5 ml per animal (see RU 2,400,822).

2. Twenty-four hours after the injection (against a background of 24-h starvation with free access to water), the animals were subcutaneously injected with alloxan trihydrate (La Chema, Czech Republic) in the form of a 4% solution in 0.9% NaCl at a single dose of 200 mg/kg [Volchegorsky I. A., Dolgushin I. I., Kolesnikov O. L., Tseilikman V. E. Experimental Modeling and Laboratory Evaluation of Adaptive Responses of the Body. Chelyabinsk: Chelyabinsk. Gos. Ped. Univ., 2000, pp. 167; Volchegorsky I. A. Tishevskaya N. V., Dementieva E. V. Antianemic effect of reamberin in rats with acute alloxan-induced diabetes mellitus. Eksp. Klin. Farmakol., 2008, vol. 71, no. 6, pp. 23-27].

For preventing fatal ketoacidosis, all rats received background treatment with insulin beginning from the third day after the alloxan injection [Federiuk, I. F., Casey H. M., Quinn M. J., Wood M. D., Ward W. K. Induction of type-1 diabetes mellitus in laboratory rats by use of alloxan: Route of administration, pitfalls, and insulin treatment. Comp. Med., 2004, vol. 54, no. 3, pp. 252-257]. For this purpose, the animals were subcutaneously injected with biphasic insulin aspart (NovoMix® 30 Penfill®, Novo Nordisk, Denmark) once a day at a dose of 3 IU/kg. The treatment was stopped if the blood glucose concentration decreased below 10 mM/l.

Fourteen days after the alloxan injection, before the administration of the RNA preparations, the blood glucose concentration varied between 19 and 24 mM/l.

### Example 1

### Stimulation of Bone Marrow Erythropoiesis in Cultured EIs with the Total RNA Preparation Isolated from Spleen Lymphoid Cells (RNAs-2)

The study was performed on 36 outbred white rats (24 erythrocyte concentrate donors of both sexes weighing 280-315 g and 12 female recipients weighing 100-120 g). The model of suppressed erythropoiesis is described in detail in the “Materials and Methods” (see “Model (I) of post-transfusion polycythemia”).

Analysis of cultured BM EIs from intact rats (model (II) of physiologically normal erythropoiesis, see the “Materials and Methods”) showed that addition of the RNAs-1 or RNAs-2 preparation to a concentration of 2 μg/ml culture medium did not substantially affect the number of EIs on Petri dishes; their number did not differ significantly from the background or control level after 24 h of cultivation. However, the qualitative composition of EI cultures was changed upon addition of the RNAs-2 preparation derived from spleen lymphoid cells of anematized rats that had a stimulatory effect on erythropoiesis (in the given case, 17 h after the blood loss (donor interval)). RNAs-2, but not RNAs-1 (derived from spleen lymphoid cells of intact rats), significantly stimulated the formation of reconstructing EIs in the culture, which indicates de repeto erythropoiesis activation in vitro. According to the authors of this method, erythropoiesis reconstruction is the first response to the stimulation both in vitro and in vivo (Table 1).

TABLE 1
Effects of the total RNA preparations derived from spleen lymphoid cells
of intact (RNAs-1) and anematized (RNAs-2) animals on the qualitative composition
of 24-h cultures of EIs from the bone marrow of intact rats

| | Parameter | | | | |
|---|---|---|---|---|---|
| | Class 1 | Class 2 | Class 3 | Inv. | Rec. |
| Group | EIs | EIs | EIs | EIs | EIs |
| Background | 79.1 ± 6.6 | 116.8 ± 7.6 | 388.1 ± 13.8 | 765.3 ± 12.7 | 122.2 ± 12.3 |
| Control | 87.7 ± 9.2 | 107.4 ± 8.2 | 373.8 ± 10.6 | 781.2 ± 13.1 | 136.4 ± 7.4 |
| (without | (p = 0.2152) | (p = 0.1683) | (p = 0.4673) | (p = 0.5273) | (p = 0.0997) |
| preparation) | | | | | |
| RNAs-1 | 83.8 ± 12.3 | 98.6 ± 11.9 | 372.4 ± 14.1 | 775.0 ± 9.6 | 125.6 ± 15.1 |
| | (p = 0.3113) | (p = 0.4124) | (p = 0.2143) | (p = 0.5914) | (p = 0.8691) |
| RNAs-2 | 93.6 ± 8.2 | 98.2 ± 13.1 | 363.4 ± 7.7 | 718.0 ± 8.5* | 184.0 ± 7.9* |
| | (p = 0.0679) | (p = 0.1287) | (p = 0.1411) | (p = 0.0281)* | (p = 0.0119)* |

Notes:
*Significant difference from the control cultures (p < 0.05).

The increase in the number of reconstructing EIs in cultures treated with RNAs-2 (from lymphoid cells of rats with activated erythropoiesis) was accompanied by a decrease in the number of involuting EIs, because their central macrophages were reinvolved in erythropoiesis, and involuting EIs were transformed into reconstructing EIs. The EI maturation was substantially accelerated, as evidenced by the data on erythropoiesis rate (Table 2). The increase in the number of reconstructing EIs was 33%, and the decrease in the number of involuting ones was slightly less than 10%; so these data unambiguously demonstrate a stimulatory effect of the RNAs-2 preparation derived from spleen lymphoid cells of the rats subjected to bleeding at the stage when these cells had a stimulatory effect on hematopoiesis (i.e., in the first phase of the response to blood loss). Erythropoiesis was significantly enhanced only in the cultures treated with RNAs-2, whereas the RNAs-1 preparation derived from spleen lymphoid cells of intact rats had no effect.

TABLE 2
Effects of the total RNA preparations RNAs-1 and RNAs-2 on the
rate of development of EIs from the bone marrow of intact rats

| | Parameter | | |
|---|---|---|---|
| | Index of CFU-e | Index of | Index of macrophage |
| | involvement in | erythroid cell | reinvolvement in |
| Group | differentiation | maturation in EIs | erythropoiesis |
| Back- | 13.26 ± 1.18 | 4.21 ± 0.51 | 0.18 ± 0.06 |
| ground | | | |
| Control | 13.43 ± 1.13 | 4.19 ± 0.60 | 0.19 ± 0.04 |
| RNAs-1 | 13.85 ± 0.99 | 4.57 ± 0.58 | 0.18 ± 0.09 |
| RNAs-2 | 17.36 ± 1.10*^{▪} | 3.36 ± 0.44*^{▪} | 0.25 ± 0.05*^{▪} |

Notes:
*Significant difference from the control cultures;
^{▪}significant difference from the cultures treated with RNAs-1 (p < 0.05).

To test this result, we performed additional experiments with higher doses of the RNA preparations (4 μg/ml culture medium). In addition, we attempted to test the activity of the stimulatory preparation in cultured EIs from the bone marrow of rats in which the proliferation of hematopoietic cells was suppressed as a result of experimental polycythemia (see “Model (I) of post-transfusion polycythemia” in the “Materials and Methods”). Control cultures were treated with erythropoietin alone.

Table 3 shows the results of these experiments. As evident from Table 3, cultured EIs from polycythemic rats were highly sensitive to RNAs-1 and RNAs-2 at doses of 4 μg/ml culture medium: as early as after 24 h of cultivation, the qualitative composition of EIs was practically the same as that of cultured EIs from intact animals; i.e., these experimental cultures exhibited physiologically normal erythropoiesis. Moreover, the number of reconstructing EIs in cultures treated with RNAs-2 was significantly greater than their number in cultures of EIs from intact animals, which clearly indicates the true stimulation of erythropoiesis, since this activation was accompanied by an increase in contacts of central macrophages of EIs with CFU-e.

TABLE 3
Effects of the total RNA preparations derived from spleen lymphoid cells of intact (RNAs-1) and
anematized (RNAs-2) animals on erythropoiesis in 24-h cultures of EIs from polycythemic rats

| | Group | | | |
|---|---|---|---|---|
| | Background | Control | | |
| | (EIs from | (EIs from | | |
| Parameter | intact rats) | polycythemic rats) | RNAs-1 | RNAs-2 |
| EIs/cm², abs. number | 1426.4 ± 15.4 | 907.1 ± 10.4^{□} | 1165.5 ± 20.3* | 1209.8 ± 34.6* |
| Class 1 EIs, % | 6.4 ± 0.5 | 2.8 ± 0.3^{□} | 6.8 ± 0.9* | 8.7 ± 1.1* |
| Class 2 EIs, % | 7.1 ± 0.4 | 4.3 ± 0.6^{□} | 8.9 ± 1.6* | 11.3 ± 2.1* |
| Class 3 EIs, % | 25.2 ± 2.7 | 26.5 ± 2.1 | 22.5 ± 3.4 | 29.2 ± 6.9 |
| Inv. EIs, % | 50.8 ± 2.5 | 59.6 ± 3.4 | 47.0 ± 4.8* | 36.2 ± 5.7*^{▪} |
| Rec. EIs, % | 11.4 ± 1.3 | 5.6 ± 0.2^{□} | 10.7 ± 2.5* | 15.8 ± 1.6*^{▪} |
| EIs with lymphoid cells | 17.4 ± 0.9 | 14.3 ± 2.6 | 25.4 ± 1.9*^{□} | 26.3 ± 2.2*^{□} |
| in the “crown,” % | | | | |

Notes:
*Significant difference from the control cultures;
^{▪}significant difference from the cultures treated with RNAs-1;
^{□}significant difference from the cultures of EIs isolated from intact rats (background) (p < 0.05).

Thus, the RNAs-1 and RNAs-2 preparations have been demonstrated to stimulate erythropoiesis, the stimulation being especially distinct in the model (I) of post-transfusion polycythemia with strong suppression of erythropoiesis, where the preparations according to the invention were even capable of overcoming this suppression to stimulate erythropoiesis.

The results obtained in the experiments on EI cultures have been further confirmed and detailed in experiments in vivo.

### Example 2

### Effects of Spleen RNA Preparations on Erythropoiesis in Polycythemic Rats In Vivo

The animas with induced polycythemia had significantly higher erythrocyte count, hemoglobin content, and hematocrit, as well as a three times lower reticulocyte count, in the peripheral blood compared to the initial levels. This shows the effectiveness of the erythropoiesis suppression model used (Table 4) and agrees with data obtained earlier by us and other researchers [Melnikov I. Yu. Study of bone marrow erythroblastic islets at different functional states of erythropoiesis. Cand. Sci. (Med.) Dissertation. Chelyabinsk, 1987, 200 pp.; Rassokhin A. G., Kruglov D. G., Zakharov Yu. M. The state of erythropoiesis and functional characteristics of bone marrow erythroblastic islets upon stimulation and inhibition of erythropoiesis. Vestn. Ross. Akad. Med. Nauk, 2000, no. 2, pp. 9-14; Tishevskaya N. V. The time course of glycosaminoglycan composition at different states of erythropoiesis in erythroblastic islets. Cand. Sci. (Med.) Dissertation. Chelyabinsk, 1995, 112 pp.; Moiseeva O. I. Physiological Mechanisms of Erythropoiesis Control. Leningrad: Nauka, 1985, 183 pp.; Filimonov V. I. Mechanisms of erythropoiesis inhibition. Patol. Fiziol. Eksp. Ter., 1972, vol. 16, no. 5, pp. 33-37; Gitelzon I. I., Sidko S. F. Cell population mechanisms of erythropoiesis suppression in polycythemia. Tsitologiya, 1977, vol. 19, no. 6, pp. 632-638; Rassokhin A. G. Effect of erythropoietin on erythropoiesis in bone marrow erythroblastic islets in polycythemia. In: Proc. II Congr. of Physiologists of the Urals, 1990, pp. 29-30].

Intravenous (i.v.) injection of the RNAs-2 preparation to rats with post-transfusion polycythemia led to a significant decrease in the erythrocyte, hemoglobin, and hematocrit levels in peripheral blood compared to the control group: the hemoglobin concentration and hematocrit were decreased on day 3 after injection; on days 4-5, both these parameters and the erythrocyte count were decreased.

Data on the effect of the RNA preparations according to the invention on the peripheral blood reticulocyte count deserve special attention. A significant increase in the reticulocyte count was detected as early as day 2 after the RNAs-2 injection; at later time points, this parameter was significantly increased not only in the rats injected with RNAs-2, but also in those injected with RNAs-1.

It is noteworthy that the increase in the peripheral blood reticulocyte count was accompanied by a steady decrease in the hemoglobin content and hematocrit (the decrease eventually becoming significant) and a significant decrease in the erythrocyte count in the rats that received RNAs-2. This indicates that the stimulation of erythropoiesis resulting from the activation of EI macrophages reflects a general activation of the entire lymphoid-macrophage system of the body, which enhances the destruction and phagocytosis of the transfused allogeneic erythrocytes. Therefore, the erythrocyte count at this stage is likely to reflect mainly the endogenous erythrocyte production.

TABLE 4
Effects of the total RNA preparations derived from spleen
lymphoid cells of intact (RNAs-1) and anematized (RNAs-2)
animals on peripheral blood parameters of polycythemic rats

| | Parameter | | | |
|---|---|---|---|---|
| | Erythrocytes | Hemoglobin | Hematocrit | Reticulocytes |
| Group | (×10¹²/l) | (g/l) | (%) | (‰) |
| Intact rats (n = 15) | 8.2 ± 0.9 | 168.4 ± 17.3 | 46.4 ± 2.7 | 44.2 ± 7.9 |
| Background (day 5 | 9.8 ± 0.7 | 244.1 ± 9.7 | 68.4 ± 3.6 | 15.0 ± 4.5 |
| of polycythemia) | | | | |

Day 1 after RNA injection

| | | | | |
|---|---|---|---|---|
| Control | 9.7 ± 1.1 | 243.5 ± 14.6 | 68.5 ± 6.8 | 11.6 ± 3.1 |
| RNAs-2 | 9.3 ± 0.5 | 236.2 ± 10.1 | 69.3 ± 2.7 | 17.0 ± 4.4 |
| RNAs-1 | 9.7 ± 0.4 | 240.2 ± 12.5 | 68.6 ± 4.2 | 15.8 ± 5.6 |

Day 2 after RNA injection

| | | | | |
|---|---|---|---|---|
| Control | 9.5 ± 0.6 | 241.4 ± 11.9 | 66.6 ± 8.2 | 12.5 ± 2.7 |
| RNAs-2 | 8.2 ± 0.1 | 230.4 ± 9.9 | 58.8 ± 2.2 | 24.5 ± 3.8*^{▪} |
| RNAs-1 | 9.3 ± 0.5 | 239.1 ± 14.0 | 64.2 ± 5.1 | 17.7 ± 4.2 |

Day 3 after RNA injection

| | | | | |
|---|---|---|---|---|
| Control | 9.5 ± 0.8 | 239.6 ± 13.3 | 64.8 ± 4.7 | 13.6 ± 1.9 |
| RNAs-2 | 7.8 ± 0.4 | 196.3 ± 9.5* | 50.2 ± 3.6* | 31.3 ± 4.0*^{▪} |
| RNAs-1 | 8.9 ± 0.7 | 220.7 ± 10.5 | 60.6 ± 4.8 | 20.3 ± 3.7* |

Day 4 after RNA injection

| | | | | |
|---|---|---|---|---|
| Control | 9.4 ± 0.7 | 236.1 ± 10.5 | 62.2 ± 4.2 | 15.1 ± 2.8 |
| RNAs-2 | 7.1 ± 0.2*^{▪} | 174.7 ± 10.1* | 46.5 ± 3.7* | 33.8 ± 4.4* |
| RNAs-1 | 8.9 ± 0.4 | 216.8 ± 11.6 | 60.4 ± 3.9 | 26.5 ± 6.1* |

Day 5 after RNA injection

| | | | | |
|---|---|---|---|---|
| Control | 9.0 ± 0.5 | 232.7 ± 10.4 | 61.9 ± 3.5 | 15.8 ± 3.2 |
| RNAs-2 | 7.0 ± 0.3*^{▪} | 157.8 ± 8.6*^{▪} | 45.7 ± 3.4* | 35.6 ± 3.1* |
| RNAs-1 | 8.8 ± 0.3 | 200.9 ± 13.3 | 54.5 ± 4.7 | 32.4 ± 5.5* |

Notes:
*Significant difference from the control group;
^{▪}significant difference from the group treated with RNAs-1 (p < 0.05).

The distinct changes in the peripheral blood reflecting an activated erythroid hematopoiesis were accompanied by changes in the erythroid tissue of the bone marrow observed on the tenth day after erythrocyte concentrate transfusion (fifth day after the injection of the RNA preparations) (Table 5). Both RNA preparations stimulated erythropoiesis in EIs: in both RNAs-1 and RNAs-2 groups, the absolute number of EIs in the bone marrow was significantly increased five days after the RNAs injection, and class 1 EIs appeared as a result of the interaction between free bone-marrow macrophages and CFU-e. The RNA preparations also accelerated erythropoiesis reconstruction: the number of EIs involved in the repeated “wave” of erythropoiesis (reconstructing EIs) was significantly increased. In addition to stimulation of proliferative processes, the RNA preparations according to the invention induced an increase in the number of mature class 3 EIs in the bone marrow and a significant decrease in the number of involuting EIs, which also indicated a more intense development of the erythroid hematopoietic lineage.

The effects of the two RNA preparations on the formation and development of BM EIs had the same direction, but they differed in strength. The RNAs-2 preparation derived from the spleen of anematized animals stimulated the development of erythroid tissue in the bone marrow more strongly, with the result that erythropoiesis in the bone marrow was restored almost to the initial level.

TABLE 5
Effects of the total RNA preparations derived from spleen lymphoid
cells of intact (RNAs-1) and anematized (RNAs-2) animals on
erythropoiesis in the bone marrow of polycythemic rats

| | Group | | |
|---|---|---|---|
| Parameter | Control | RNAs-1 | RNAs-2 |
| EIs, abs. number | 115.5 ± 12.6 | 201.6 ± 11.2* | 223.2 ± 10.9* |
| (10³/femoral bone) | | | |
| Class 1 EIs, % | 0 | 2.4 ± 0.1* | 6.8 ± 0.3*^{▪} |
| Class 2 EIs, % | 2.1 ± 0.3 | 6.5 ± 0.9* | 10.5 ± 1.4*^{▪} |
| Class 3 EIs, % | 12.7 ± 1.8 | 27.2 ± 3.5* | 30.6 ± 5.1* |
| Inv. EIs, % | 78.8 ± 8.3 | 53.4 ± 5.6* | 32.9 ± 4.2*^{▪} |
| Rec. EIs, % | 6.4 ± 1.1 | 11.7 ± 1.3* | 21.5 ± 2.8*^{▪} |

Notes:
*Significant difference from the control cultures;
^{▪}significant difference from the cultures treated with RNAs-1 (p < 0.05).

Thus, the data presented above allow us to conclude that the tested RNA preparations derived from spleen lymphoid cells have a stimulatory effect not only on a physiologically normal erythropoiesis, but also on a suppressed one.

### Example 3

### Suppression of Erythropoiesis by RNA Preparations from Spleen Lymphoid Cells Isolated at a Certain Stage after Acute Blood Loss

To demonstrate an inhibitory activity of the total RNA preparation from spleen lymphoid cells isolated at a certain stage of recovery, namely in its second phase, is important not only for understanding how the morphogenetic function of lymphocytes and their capacity for transmitting regeneration information are controlled, but also for developing the strategy of RNA therapy of severe autoimmune diseases and so-called proliferative diseases or hyperproliferation conditions.

The inhibitory RNA preparation according to the invention (RNAs-3) was derived from spleen lymphocytes isolated four days after acute blood loss, which are known to suppress hematopoiesis. We estimated the strength of the inhibitory effect of RNAs-3 in experiments on cultured BM EIs of both intact rats (model (II) of physiologically normal erythropoiesis) and rats with hematopoiesis stimulated by acute blood loss (model (III) of compensatory erythropoiesis, see the “Materials and Methods”). To assess the activity of this preparation more comprehensively, we also estimated the RNAs-3 inhibitory activity in experiments on BM EIs that were not only isolated from rats with hematopoiesis stimulated by acute blood loss, but also cultured in the presence of an increased concentration of erythropoietin as an additional stimulator of erythropoiesis (both experimental models were variants of model (III) of compensatory erythropoiesis). Thus, in this last case, RNAs-3 could suppress erythropoiesis only if it were to overcome both the hematopoiesis stimulation by acute blood loss in vivo and the additional in vitro stimulation of the maturation of EIs belonging to proliferating classes (class 1, class 2, and Rec. EIs).

In addition, we estimated the inhibitory activity of RNAs-3 in vivo, in experiments on animals whose hematopoiesis was stimulated by acute blood loss. The preparation was injected intravenously 1 h after the blood loss at a single dose of 30 μg/100 g body weight.

The results of the experiments are shown in Tables 6, 7, and 8.

We used EI cultures containing the following doses of the RNAs-3 preparation and erythropoietin:
0.5 IU/ml of erythropoietin and 4 μg/ml of RNAs-3 (five cultures);
0.5 IU/ml of erythropoietin and 2 μg/ml of RNAs-3 (five cultures);
1.5 IU/ml of erythropoietin and 4 μg/ml of RNAs-3 (five cultures);
1.5 IU/ml of erythropoietin and 2 μg/ml of RNAs-3 (five cultures);
0.5 IU/ml of erythropoietin (control);
1.5 IU/ml of erythropoietin (control).

As evident from Table 6, the presence of the RNAs-3 preparation in the culture of EIs suppressed the development of erythroid cells. The inhibitory effect of 4 μg/ml RNAs-3 in model (II) of physiologically normal erythropoiesis was somewhat stronger than that of 2 μg/ml RNAs-3 (at the same concentration of erythropoietin of 0.5 IU/ml) (Table 6). Estimation of the proportions of EIs of different maturation classes showed that the inhibition of erythropoiesis was associated with the suppression of the CFU-e and proerythroblast attachment to the plasma membrane of the central macrophages, as evidenced by a decrease in the number of EIs of proliferating classes (class 1, class 2, and Rec. EIs). The suppression of differentiation and maturation of erythroid cells was also confirmed by an increase in the number of class 3 EIs, probably, because the maturation and denucleation of oxyphilic normoblasts and the release of reticulocytes from the EI “crown” were hampered.

The effect of an increased dose of the RNAs-3 preparation was somewhat stronger but did not differ substantially from the effect of the lower dose.

TABLE 6
Effect of the RNAs-3 preparation derived from spleen lymphoid
cells on the culture of EIs from the bone marrow of intact
rats (model (II) of physiologically normal erythropoiesis)

| | Group | | |
|---|---|---|---|
| | | Experiment 1: | Experiment 2: |
| | Control: | 0.5 IU/ml EP + | 0.5 IU/ml EP + |
| Parameter | 0.5 IU/ml EP | 2 μg/ml RNAs-3 | 4 μg/ml RNAs-3 |
| EIs/cm², abs. | 1443.6 ± 43.7 | 1427.2 ± 36.9 | 1401 ± 25.5 |
| number | | | |
| Class 1 EIs, % | 6.6 ± 0.3 | 4.0 ± 0.5* | 3.4 ± 0.2* |
| Class 2 EIs, % | 8.8 ± 0.9 | 6.1 ± 0.4 | 5.3 ± 0.4* |
| Class 3 EIs, % | 23.1 ± 4.5 | 28.6 ± 3.9 | 36.3 ± 5.7* |
| Inv. EIs, % | 51.5 ± 5.8 | 54.7 ± 4.8 | 50.5 ± 7.4 |
| Rec. EIs, % | 12.1 ± 1.2 | 7.3 ± 1.1* | 5.8 ± 2.2* |

Notes:
EP, erythropoietin;
*significant difference from the control cultures (p < 0.05).

In model (III) of compensatory erythropoiesis, only the higher dose of the RNAs-3 preparation suppressed the development of erythroid cells (Table 7). The presence of 4 μg/ml RNAs-3 in the culture medium reduced the capacity of the central macrophages for forming new EIs both de novo and de repeto, as evidenced by a decrease in the numbers of class 1 and Rec. EIs in the culture. The weaker inhibitory effect of RNAs-3 on compensatory erythropoiesis than on physiologically normal one was probably related to an imbalance between the factors stimulating and inhibiting the erythroid lineage, because the addition of a large amount of erythropoietin to the culture medium shifted the balance towards erythropoiesis stimulation.

TABLE 7
Effect of the RNAs-3 preparation derived from spleen lymphoid
cells on the culture of EIs from the bone marrow of anematized
rats (model (III) of compensatory erythropoiesis)

| | Group | | |
|---|---|---|---|
| | | Experiment 1: | Experiment 2: |
| | Control: | 1.5 IU/ml EP + | 1.5 IU/ml EP + |
| Parameter | 1.5 IU/ml EP | 2 μg/ml RNAs-3 | 4 μg/ml RNAs-3 |
| EIs/cm², abs. | 1501.3 ± 33.5 | 1489.2 ± 27.7 | 1422 ± 19.8 |
| number | | | |
| Class 1 EIs, % | 9.76 ± 1.2 | 7.4 ± 0.6 | 5.0 ± 0.3* |
| Class 2 EIs, % | 10.1 ± 1.6 | 8.5 ± 0.7 | 7.3 ± 1.1 |
| Class 3 EIs, % | 27.6 ± 3.4 | 26.3 ± 4.2 | 30.7 ± 4.2 |
| Inv. EIs, % | 47.8 ± 6.3 | 48.5 ± 5.8 | 49.6 ± 5.3 |
| Rec. EIs, % | 15.9 ± 2.1 | 11.4 ± 2.1 | 6.6 ± 1.2* |

Notes:
EP, erythropoietin;
*significant difference from the control cultures (p < 0.05).

Microscopic examination of EIs cultured in the presence of 0.5 IU/ml of erythropoietin (model (II) of physiologically normal erythropoiesis) and 4 μg/ml of the RNAs-3 preparation showed an unusually high frequency of contacts between class 3 EIs and lymphoid cells. Therefore, we analyzed the numbers of the contacts of lymphoid cells with the “crowns” of EIs belonging to different maturation classes. Earlier, we performed similar estimations when studying the effects of erythropoietin at different doses on the erythropoiesis rate in vitro and found that the EIs whose “crown” contains intensely proliferating cells (class 1 and class 2 EIs) form the greatest numbers of contacts with lymphoid cells. In this case, however, in the presence of the RNAs-3 preparation in the culture medium, class 3 EIs formed the greatest number of contacts, which undoubtedly deserved special attention.

TABLE 8
Effect of the RNAs-3 preparation on the percentage of EIs
with lymphoid cells in the “crown” under the conditions
of erythropoietin-stimulated erythropoiesis in vivo

Group

| Parameter | 1.5 IU/ml EP | 1.5 IU/ml EP + 4 μg/ml RNAs-3 |
|---|---|---|
| Class 1 EIs, % | 20.6 ± 2.6 | 15.8 ± 4.7 |
| Class 2 EIs, % | 11.5 ± 2.3 | 8.1 ± 2.2 |
| Class 3 EIs, % | 9.1 ± 1.8 | 26.5 ± 3.4* |
| Inv. EIs, % | 6.4 ± 1.5 | 8.6 ± 1.9 |
| Rec. EIs, % | 25.5 ± 5.1 | 19.6 ± 4.5 |

Notes:
EP, erythropoietin;
*significant difference from the control cultures (p < 0.05).

The results of in vivo experiments entirely agreed with the data on BM EI cultures. Intravenous injection of the RNAs-3 preparation, but not the injection of the same volume (0.1 ml) of physiological saline (control), to rats 1 h after blood loss (model (III) of compensatory erythropoiesis) led to a significant decrease in the reticulocyte count of the peripheral blood (Table 9), as well as a significant decrease in the numbers of class 1 and Rec. EIs and an increase in the number of Inv. EIs in the bone marrow (Table 10).

TABLE 9
Changes in the peripheral blood parameters in rats injected with
the RNAs-3 preparation against a background of acute post-hemorrhagic
anemia (model (III) of compensatory erythropoiesis)

Group

| Parameter | Experimental group | Control group |
|---|---|---|
| Erythrocytes (×10¹²/l) | 5.8 ± 0.9 | 6.6 ± 0.5 |
| Hemoglobin (g/l) | 148.4 ± 11.6 | 157.2 ± 10.3 |
| Hematocrit (%) | 41.6 ± 1.9 | 42.5 ± 1.4 |
| Reticulocytes (‰) | 38.1 ± 4.8* | 56.3 ± 6.6 |

Notes:
*Significant difference from the control cultures (p < 0.05).

TABLE 10
Changes in the EI composition in the bone marrow
of the rats injected with the RNAs-3 preparation
against a background of acute post-hemorrhagic anemia
(model (III) of compensatory erythropoiesis)

Group

| Parameter | Experimental group | Control group |
|---|---|---|
| EIs, abs. number | 324.4 ± 11.2 | 345.6 ± 16.7 |
| (10³/femoral bone) | | |
| Class 1 EIs, % | 9.7 ± 1.1* | 14.9 ± 1.7 |
| Class 2 EIs, % | 28.8 ± 4.2 | 33.3 ± 5.8 |
| Class 3 EIs, % | 115.9 ± 10.2 | 98.3 ± 9.6 |
| Inv. EIs, % | 188.5 ± 12.4* | 141.4 ± 10.2 |
| Rec. EIs, % | 41.3 ± 5.5* | 63.5 ± 4.1 |

Notes:
*Significant difference from the control cultures (p < 0.05).

Thus, all experiments described in Examples 1, 2, and 3 unambiguously demonstrate that the total RNA preparations derived from lymphoid cells preserve all the main regulatory activities of these cells and in full compliance with them perform their regulatory function in respect of the processes of proliferation and differentiation of hematopoietic cells.

Further studies were aimed at answering the following questions:
(1) Can the stimulatory preparation RNAs-2 cure severe chronic anemia and, hence, be used instead of periodic blood transfusions used for this purpose?
(2) Does the RNAs-2 preparation affect hematopoietic lineages other than the erythropoietic one?
(3) Do total RNA preparations isolated from other lymphoid organs (the thymus and bone marrow) and peripheral blood lymphocytes possess these regulatory properties?
(4) Do the RNA preparations isolated from the bone marrow of intact rats have therapeutic properties?

The effects of the total RNA-1 and RNA-2 preparations were estimated using two experimental models: model (IV) of benzene-induced chronic hypoplastic anemia and model (V) of sub-lethal irradiation (see “Materials and Methods”).

### Example 4

### Effects of the Total RNAs-1 and RNAs-2 Preparations on Erythropoiesis in Rats with Benzene-Induced Chronic Hypoplastic Anemia (Model IV)

Chronic hypoplastic anemia was induced by three subcutaneous injections of a mixture of benzene with sterile vegetable oil. Control rats were injected with the same amount of pure vegetable oil (for details, see “Materials and Methods”).

Eighteen rats were used in the experiment. Administration of the RNAs-1 and RNAs-2 preparations was started 28 days after the last injection of benzene; they were injected intravenously at a dose of 15 μg/100 g body weight three times at ten-day intervals (thus, the total dose was 45 μg/100 g body weight).

Table 11 shows the time course of the changes in the peripheral blood cell counts. These data demonstrate that benzene caused a significant decrease in the counts of cells originating from all hematopoietic lineages; the reticulocyte count was decreased by a factor of 7; the leukocyte and platelet counts, by a factor of 4.5.

TABLE 11
Peripheral blood cell counts in the course
of benzene-induced anemia in rats

| | Parameter | | | |
|---|---|---|---|---|
| | Erythrocytes | Reticulocytes | Leukocytes | Platelets |
| Group | (×10¹²/l) | (‰) | (×10⁹/l) | (×10⁹/l) |
| Back- | 9.8 ± 1.5 | 46.7 ± 3.2 | 11.5 ± 1.1 | 468.6 ± 23.2 |
| ground | | | | |
| (intact | | | | |
| rats) | | | | |

Day after the third benzene injection

| | | | | |
|---|---|---|---|---|
| Day 7 | 8.4 ± 1.3 | 10.5 ± 1.8^{□} | 3.6 ± 1.0^{□} | 188.3 ± 10.2^{□} |
| Day 14 | 8.0 ± 1.3 | 8.3 ± 0.8^{□} | 3.2 ± 0.6^{□} | 139.8 ± 12.5^{□} |
| Day 21 | 7.9 ± 0.8 | 6.1 ± 0.4^{□} | 3.5 ± 0.5^{□} | 126.2 ± 10.1^{□} |
| Day 28 | 6.7 ± 0.4^{□} | 5.9 ± 1.1^{□} | 2.6 ± 0.4^{□} | 93.1 ± 7.9^{□} |

Notes:
^{□}Significant difference from the background value (Kolmogorov-Smirnov test, p < 0.05).

The animals were divided into three equal groups:
The control group (rats with benzene-induced anemia not treated with an RNA preparation).
The RNAs-1 group (rats injected with the total RNA preparation derived from the spleen of intact rats).
The RNAs-2 group (rats injected with the total RNA preparation derived from the spleen of rats 17 h after blood loss).

The RNA preparations were injected intravenously three times at ten-day intervals. Control animals were injected with the same volume of 0.9% NaCl on the same days. The initial concentration of the total RNA preparations from lymphoid cells of intact and anematized animals was 3 μg/μl.

The effects of the RNA preparations from spleen lymphoid cells on the state of the peripheral blood in the rats with benzene-induced anemia were estimated every nine to ten days (Table 12). The first injection of RNAs-2 led to a significant twofold increase in the reticulocyte count on day 10. After the second injection of this preparation, the reticulocyte count was increased by a factor of 3, and the number of platelets was increased by a factor of 1.4. After the third injection of RNAs-2, the reticulocyte count continued to increase and became five times higher than the control value. By the 18th day after the injection of the RNAs-2 preparation, the stimulation of erythrocyte production by the bone marrow was reflected in the blood erythrocyte count, which became significantly higher than in the control group.

TABLE 12
Peripheral blood cell counts in rats with benzene-induced anemia
(model IV) after injection of the RNAs-1 and RNAs-2 preparations

| | Parameter | | | |
|---|---|---|---|---|
| | Erythrocytes | Reticulocytes | Leukocytes | Platelets |
| Group | (×10¹²/l) | (‰) | (×10⁹/l) | (×10⁹/l) |
| Intact rats | 9.8 ± 1.5 | 46.7 ± 3.2 | 11.5 ± 1.1 | 468.6 ± 23.2 |
| | (7-10) | (35-50) | (8-15) | (350-800) |
| Background | 6.7 ± 0.4 | 5.9 ± 1.1 | 2.6 ± 0.4 | 93.1 ± 7.9 |
| (benzene- | | | | |
| induced anemia) | | | | |

Day 10 after the first RNA injection

| | | | | |
|---|---|---|---|---|
| Control | 6.4 ± 0.4 | 6.1 ± 0.8 | 2.5 ± 0.5 | 94.2 ± 6.3 |
| RNAs-1 | 6.4 ± 0.7 | 9.9 ± 1.2 | 2.7 ± 0.4 | 100.3 ± 5.8 |
| RNAs-2 | 6.5 ± 0.5 | 12.4 ± 2.3*^{□} | 2.7 ± 0.5 | 118.7 ± 10.2 |

Day 10 after the second RNA injection

| | | | | |
|---|---|---|---|---|
| Control | 6.6 ± 0.5 | 6.3 ± 0.8 | 2.6 ± 0.6 | 89.3 ± 9.1 |
| RNAs-1 | 6.8 ± 0.7 | 8.8 ± 0.9^{□} | 8.9 ± 0.3*^{□} | 104.5 ± 8.9 |
| RNAs-2 | 7.0 ± 0.8 | 18.5 ± 1.2*^{▪□} | 3.0 ± 0.5^{▪} | 127.8 ± 9.2* |

Day 9 after the third RNA injection (day 29 of treatment)

| | | | | |
|---|---|---|---|---|
| Control | 5.9 ± 0.7 | 5.9 ± 0.6 | 2.8 ± 0.6 | 90.9 ± 7.5 |
| RNAs-1 | 5.8 ± 0.9 | 10.9 ± 0.7^{□} | 9.3 ± 0.7*^{□} | 109.9 ± 7.7 |
| RNAs-2 | 7.2 ± 0.5 | 26.1 ± 1.4*^{▪□} | 3.2 ± 0.1^{▪} | 140.3 ± 9.6*^{▪□} |

Day 18 after the third RNA injection (day 38 of treatment)

| | | | | |
|---|---|---|---|---|
| Control | 5.5 ± 0.4 | 4.1 ± 0.3 | 2.9 ± 0.5 | 106.1 ± 9.4 |
| RNAs-1 | 6.0 ± 0.3 | 11.2 ± 0.5*^{□} | 10.0 ± 0.4*^{□} | 122.4 ± 8.1 |
| RNAs-2 | 7.8 ± 0.5* | 29.2 ± 1.1*^{▪□} | 3.7 ± 0.3^{▪} | 177.5 ± 10.3*^{▪□} |

Day 30 after the third RNA injection (day 50 of treatment)

| | | | | |
|---|---|---|---|---|
| Control | 4.8 ± 0.6^{□} | 4.2 ± 0.4 | 3.1 ± 0.7 | 98.6 ± 8.8 |
| RNAs-1 | 6.4 ± 0.6 | 15.5 ± 0.6*^{□} | 10.2 ± 0.8*^{□} | 150.8 ± 10.1*^{□} |
| RNAs-2 | 7.8 ± 0.5* | 30.1 ± 1.5*^{▪□} | 4.4 ± 0.4^{▪} | 214.4 ± 11.2*^{▪□} |

Day 40 after the third RNA injection (day 60 of treatment)

| | | | | |
|---|---|---|---|---|
| Control | 4.7 ± 0.3^{□} | 4.2 ± 0.5 | 3.1 ± 0.5 | 102.8 ± 8.7 |
| RNAs-1 | 6.5 ± 0.7 | 19.4 ± 1.1*^{□} | 10.1 ± 0.6*^{□} | 168.9 ± 9.9*^{□} |
| RNAs-2 | 7.8 ± 0.4* | 32.8 ± 1.6*^{▪□} | 4.9 ± 0.5^{▪□} | 225.6 ± 12.0*^{▪□} |

Day 50 after the third RNA injection (day 70 of treatment)

| | | | | |
|---|---|---|---|---|
| Control | 5.3 ± 0.5 | 6.7 ± 1.1 | 3.8 ± 0.4 | 124.5 ± 11.3 |
| RNAs-1 | 6.9 ± 0.8 | 18.5 ± 2.3*^{□} | 11.3 ± 1.2*^{□} | 170.7 ± 10.1*^{□} |
| RNAs-2 | 7.9 ± 0.9* | 35.1 ± 2.2*^{▪□} | 6.3 ± 1.3^{▪□} | 219.9 ± 9.9*^{▪□} |

Day 62 after the third RNA injection (day 82 of treatment)

| | | | | |
|---|---|---|---|---|
| Control | 5.6 ± 0.6 | 7.2 ± 0.8 | 4.3 ± 0.5 | 139.7 ± 15.6^{□} |
| RNAs-1 | 6.5 ± 0.4 | 20.2 ± 3.1*^{□} | 10.2 ± 0.8* | 183.2 ± 12.8*^{□} |
| RNAs-2 | 7.7 ± 0.8* | 36.6 ± 2.7*^{▪□} | 8.8 ± 1.6* | 226.9 ± 13.3*^{□} |

Day 70 after the third RNA injection (day 90 of treatment)

| | | | | |
|---|---|---|---|---|
| Control | 6.9 ± 0.5 | 12.1 ± 1.3^{□} | 6.7 ± 0.8^{□} | 188.9 ± 12.2^{□} |
| RNAs-1 | 6.9 ± 0.6 | 24.4 ± 2.2*^{□} | 10.3 ± 1.8^{□} | 214.1 ± 18.2^{□} |
| RNAs-2 | 7.5 ± 0.9 | 39.3 ± 2.5*^{▪□} | 10.1 ± 1.7^{□} | 239.3 ± 11.9*^{□} |

Day 80 after the third RNA injection (day 100 of treatment)

| | | | | |
|---|---|---|---|---|
| Control | 6.7 ± 0.7 | 19.1 ± 1.5^{□} | 6.5 ± 0.7^{□} | 194.4 ± 13.6^{□} |
| RNAs-1 | 7.1 ± 0.5 | 26.6 ± 2.5^{□} | 9.9 ± 1.6^{□} | 229.5 ± 14.1^{□} |
| RNAs-2 | 7.4 ± 1.2 | 38.8 ± 4.7*^{▪□} | 10.5 ± 1.9^{□} | 248.4 ± 14.8^{□} |

Day 90 after the third RNA injection (day 110 of treatment)

| | | | | |
|---|---|---|---|---|
| Control | 7.0 ± 0.5 | 27.4 ± 2.8^{□} | 6.6 ± 0.9^{□} | 223.7 ± 15.5^{□} |
| RNAs-1 | 6.8 ± 0.8 | 33.3 ± 3.7^{□} | 9.6 ± 1.3^{□} | 245.4 ± 15.7^{□} |
| RNAs-2 | 7.1 ± 1.1 | 38.2 ± 3.9^{□} | 10.5 ± 1.9*^{□} | 260.4 ± 17.3^{□} |

Notes:
*Significant difference from the control group (Kolmogorov-Smirnov test);
^{▪}significant difference from the animals treated with RNAs-1;
^{□}significant difference from the animals with anemia (background) (p < 0.05).

Forty days after the third injection of the RNAs-2 preparation to the animals with benzene-induced anemia, their reticulocyte, platelet, and erythrocyte counts were, respectively, 8, more than 2, and 1.7 times increased. In the control animals, the reticulocyte and erythrocyte counts continued decreasing at this time points, while the platelet count barely approached the values that were reached in the experimental groups as early as the 10^{th} day after the first injection of the preparations according to the invention.

The injection of the RNAs-1 preparation to the animals with benzene-induced anemia primarily affected the peripheral blood leukocyte count. This parameter was significantly increased as early as day 10 after the second RNAs-1 injection and was three times higher than the control value by day 40 of the treatment. As early as day 30 after the third RNAs-1 injection, the state of all the three hematopoietic lineages in the peripheral blood was distinctly improved: the reticulocyte, leukocyte, and platelet counts were increased, respectively, by factors of almost 4, 3, and 1.5; this clearly indicates that the total RNA preparation from spleen lymphoid cells of intact animals possesses a stimulatory activity towards hematopoiesis as a whole.

Having obtained evidence that RNAs-1 injection caused partial restoration of the leukocyte count in the blood of rats with benzene-induced anemia, we attempted to determine which types of leukocytes account for the changes in the white cell component of the peripheral blood (Table 13). The results suggest that the RNA preparation from spleen lymphoid cells of intact rats primarily stimulated the lymphoid tissue of the body intoxicated with benzene, after which the animal's own lymphocytes “supported” with exogenous RNAs-1 promoted the recovery of hematopoiesis.

TABLE 13
Percentages of different types of leukocytes in the peripheral blood of rats with benzene-
induced anemia (model IV) after injection of the RNAs-1 and RNAs-2 preparations

| | Parameter | | | | |
|---|---|---|---|---|---|
| Group | Neutrophils | Basophils | Eosinophils | Lymphocytes | Monocytes |
| Intact rats | 18.4 ± 1.3 | 0.31 ± 0.03 | 3.6 ± 0.4 | 68.5 ± 10.3 | 4.1 ± 0.4 |
| Background | 3.3 ± 1.5 | 0.06 ± 0.01 | 1.4 ± 0.2 | 90.8 ± 9.5 | 2.3 ± 0.4 |
| (benzene- | | | | | |
| induced anemia) | | | | | |

Day 9 after the third RNA injection

| | | | | | |
|---|---|---|---|---|---|
| Control | 3.2 ± 0.6 | 0.03 ± 0.01 | 1.0 ± 0.4 | 92.2 ± 6.4 | 2.2 ± 0.5 |
| RNAs-1 | 10.5 ± 0.7*^{□} | 0.04 ± 0.01 | 1.8 ± 0.6 | 85.4 ± 5.7 | 2.5 ± 0.6 |
| RNAs-2 | 4.6 ± 0.4^{▪} | 0.04 ± 0.008 | 1.1 ± 0.5 | 91.9 ± 9.8 | 1.5 ± 0.8 |

Day 18 after the third RNA injection

| | | | | | |
|---|---|---|---|---|---|
| Control | 3.8 ± 0.5 | 0.06 ± 0.01 | 1.6 ± 0.7 | 91.7 ± 6.6 | 1.9 ± 0.7 |
| RNAs-1 | 14.2 ± 1.3*^{□} | 0.10 ± 0.02*^{□} | 2.1 ± 1.0 | 80.5 ± 7.8 | 2.1 ± 0.4 |
| RNAs-2 | 6.9 ± 1.0^{▪} | 0.02 ± 0.01*^{▪} | 1.4 ± 0.5 | 87.1 ± 6.3 | 2.8 ± 1.1 |

Day 30 after the third RNA injection

| | | | | | |
|---|---|---|---|---|---|
| Control | 3.7 ± 0.4 | 0.05 ± 0.02 | 1.9 ± 0.3 | 91.5 ± 5.2 | 3.0 ± 0.5 |
| RNAs-1 | 16.8 ± 1.1*^{□} | 0.13 ± 0.02*^{□} | 1.6 ± 0.4 | 75.7 ± 5.4*^{□} | 3.4 ± 0.9 |
| RNAs-2 | 7.1 ± 1.1*^{▪□} | 0.09 ± 0.01^{▪} | 2.2 ± 0.4 | 85.3 ± 8.1 | 2.5 ± 0.3 |

Day 40 after the third RNA injection

| | | | | | |
|---|---|---|---|---|---|
| Control | 3.5 ± 0.6 | 0.08 ± 0.01 | 2.1 ± 0.8 | 92.9 ± 3.8 | 2.7 ± 0.2 |
| RNAs-1 | 17.6 ± 0.8*^{□} | 0.15 ± 0.02*^{□} | 1.7 ± 0.6 | 74.3 ± 5.5*^{□} | 3.1 ± 0.6 |
| RNAs-2 | 9.8 ± 0.8*^{▪□} | 0.14 ± 0.02*^{□} | 1.9 ± 0.9 | 82.7 ± 7.2 | 2.9 ± 0.8 |

Notes:
*Significant difference from the control group (Kolmogorov-Smirnov test);
^{▪}significant difference from the animals treated with RNAs-1;
^{□}significant difference from the animals with anemia (background) (p < 0.05).

It has been demonstrated [Zakharov V. N., Karaulov A. V., Sokolov V. V. et al. Changes in the blood system when exposed to radiation and benzene (ed. Gitelzon II). Novosibirsk: Nauka. Siberian Branch, 1990, 241 pp.] that the lymphoid tissue ensures normal cell differentiation in the course of hematopoiesis; therefore, any disturbance in the interactions of the elements of the lymphoid lineage with one another and/or with other hematopoietic lineages may trigger the hematotropic effect of benzene. It also cannot be excluded that the regulatory effects of the RNA preparations from lymphoid cells on hematopoiesis in rats with benzene-induced anemia are mediated by bone marrow macrophages.

In summary, the results of experiments with model (IV) of benzene-induced chronic hypoplastic anemia lead to the following conclusions.

The RNA preparations tested (both RNAs-1 and RNAs-2) have distinct effects not only on erythropoiesis, but also on hematopoiesis in general. The RNAs-2 preparation has a higher activity, which is expressed in its quicker and stronger effects on the reticulocyte and platelet counts at all time points of the observation, including the last one. The effects of both preparations are prolonged: the parameters of peripheral blood continued improving 40 days after the last injection of the preparations.

The effects of the RNAs-1 and RNAs-2 preparations differ from each other not only in strength, but also in specificity. This is especially noticeable at the initial stage of hematopoiesis stimulation. The RNAs-2 preparation derived from lymphoid cells stimulated with blood loss mainly promotes the restoration of erythropoiesis; less strongly, the restoration of the platelet count; and even less strongly, that of the leukocyte count. In contrast, the RNAs-1 preparation mainly affects the homologous tissue; i.e., its strongest correcting effect is targeted at the white blood cell lineage. Therefore, we suppose that both RNAs-1 and RNAs-2 preparations should be administered for more complete recovery in the cases when it is necessary to restore all hematopoietic lineages.

Two months after the start of the administration of the RNA preparations, hematopoiesis was considerably restored, although we used very small doses of the preparations. It was obvious that the doses could be increased to enhance the treatment efficiency. We took this into consideration when planning some of the subsequent experiments, including those with irradiation.

### Example 5

### Effect of the Total RNA Preparation from the Bone Marrow of Anematized Rats on Erythropoiesis in Rats with Benzene-Induced Chronic Hypoplastic Anemia (Model IV)

First, further developing the preceding experiment, we attempted to increase the efficiency of treatment and completely restore the parameters of peripheral blood and bone marrow hematopoiesis by changing the source of the preparation rather its dose.

For this purpose, we isolated the total RNA preparation from the bone marrow of anematized rats at the stage of hematopoiesis stimulation (17 h after a blood loss of 2% of the body weight) (RNAbm-2) by the same method and studied its effect on hematopoiesis in rats with benzene-induced chronic hypoplastic anemia (model IV). To that end, 4 weeks after the last benzene injection, the RNAbm-2 preparation was administered to six experimental rats, three times at ten-day intervals, at a dose of 15 μg/100 g body weight. Control animals were injected with 0.9% NaCl on the same days.

A total of 11 rats were used in the experiment. The first injection of the RNAbm-2 preparation was intravenously injected 28 days after the last benzene injection; the preparation was injected at a dose of 15 μg/100 g body weight three times at ten-day intervals (the total dose was 45 μg/100 g body weight). Blood cells were counted every 7 days. 10 days after the last RNAbm-2 injection, the experimental and control animals were euthanized to assess the erythropoiesis in the bone marrow.

The reticulocyte count in the peripheral blood was increased by a factor of three (Table 14) as early as seven days after the first RNAbm-2 injection. Seven days after the second injection, the reticulocyte, leukocyte, and platelet counts were significantly increased.

TABLE 14
Effect of the RNAbm-2 preparation derived from the bone marrow of anematized
rats on erythropoiesis in rats with benzene-induced anemia (model IV)

| | Parameter | | | |
|---|---|---|---|---|
| | Erythrocytes | Reticulocytes | Leukocytes | Platelets |
| Group | (×10¹²/l) | (‰) | (×10⁹/l) | (×10⁹/l) |
| Intact rats | 7.8 ± 1.2 | 45.9 ± 3.6 | 9.9 ± 1.3 | 447.4 ± 21.5 |
| Background | 5.2 ± 0.7 | 5.3 ± 0.5 | 2.3 ± 0.7 | 87.3 ± 12.9 |
| (20 days after the last | | | | |
| injection of benzene) | | | | |

7 days after the first RNAbm injection

| | | | | |
|---|---|---|---|---|
| Experiment | 6.1 ± 1.4 | 19.1 ± 1.3*^{□} | 4.3 ± 0.8 | 128.5 ± 15.8 |
| Control | 5.4 ± 0.6 | 6.0 ± 1.2 | 2.4 ± 0.6 | 90.8 ± 15.3 |

7 days after the second RNAbm injection

| | | | | |
|---|---|---|---|---|
| Experiment | 6.3 ± 1.1 | 28.5 ± 3.2*^{□} | 5.1 ± 0.5*^{□} | 148.7 ± 12.4*^{□} |
| Control | 5.4 ± 0.9 | 5.8 ± 0.4 | 2.3 ± 0.6 | 94.6 ± 10.1 |

7 days after the third RNAbm injection

| | | | | |
|---|---|---|---|---|
| Experiment | 6.9 ± 1.3 | 36.7 ± 4.9*^{□} | 7.5 ± 1.4*^{□} | 199.6 ± 11.5*^{□} |
| Control | 5.2 ± 0.8 | 5.9 ± 1.1 | 2.5 ± 0.3 | 92.4 ± 12.8 |

10 days after the third RNAbm injection

| | | | | |
|---|---|---|---|---|
| Experiment | 7.3 ± 1.4*^{□} | 39.5 ± 5.2*^{□} | 7.9 ± 1.9*^{□} | 212.5 ± 15.4*^{□} |
| Control | 5.2 ± 1.1 | 8.3 ± 1.8 | 3.1 ± 0.2 | 96.3 ± 10.6 |

Notes:
*Significant difference from the control group;
^{□}significant difference from the animals with anemia (background) (p < 0.05).

The data on the quantitative and qualitative compositions of bone marrow EIs showed that the administration of the RNAbm-2 preparation isolated from the bone marrow of anematized rats to animals with benzene-induced anemia completely restored the physiologically normal rate and qualitative pattern of the development of erythroid cells in the EIs (Table 15).

TABLE 15
Changes in the composition of bone marrow erythroblastic
islets in rats with benzene-induced anemia (model IV) ten
days after the last injection of the RNAbm-2 preparation

| | Group | | |
|---|---|---|---|
| | | Control | |
| | Background | (benzene-induced | Experiment |
| Parameter | (intact rats) | anemia) | (RNAbm-2) |
| EIs, abs. number | 264.5 ± 18.8 | 106.4 ± 8.8^{□} | 228.4 ± 16.1* |
| (10³/femoral bone) | | | |
| Class 1 EIs, % | 4.7 ± 0.9 | 0^{□} | 4.2 ± 0.4* |
| Class 2 EIs, % | 6.3 ± 1.2 | 2.1 ± 0.3^{□} | 5.9 ± 1.1* |
| Class 3 EIs, % | 25.3 ± 3.6 | 15.7 ± 2.5^{□} | 24.1 ± 2.8* |
| Inv. EIs, % | 51.1 ± 8.3 | 75.6 ± 7.2^{□} | 53.5 ± 5.6* |
| Rec. EIs, % | 12.6 ± 1.5 | 6.2 ± 0.8^{□} | 11.7 ± 1.6* |

Notes:
*Significant difference from the control group;
^{□}significant difference from the intact animals (background) (p < 0.05).

As seen from Table 15, the EIs of the animals treated with the RNAbm-2 preparation according to the invention did not differ from those of the intact animals in either quantitative or qualitative characteristics ten days after the last injection of the preparation.

### Example 6

### Effects of the Total RNA Preparations from the Bone Marrow and Thymus on the Restoration of the Peripheral Blood Parameters and Bone Marrow Erythropoiesis in Rats Subjected to Sub-Lethal Irradiation (Model V)

In experiments with irradiation (see “Model (V) of sub-lethal irradiation” in the “Materials and Methods”), we used total RNA preparations derived from the bone marrow and lymphoid cells of the thymus, the preparation doses being higher than in preceding experiments (30 and 20 μg/100 g body weight for the first and second injections of bone marrow RNA, respectively, and 30 μg/100 g body weight for the injection of thymic cell RNA versus 15 μg/100 g body weight in Examples 1-5). The total RNA preparations were obtained by the same method in order to compare the activity pattern and efficiency of the RNA preparations according to the invention derived from regulatory cells of different lymphoid organs.

Acute irradiation at a sub-lethal dose caused considerable changes in the quantitative composition of peripheral blood cells. On the third day after irradiation, the peripheral blood of untreated animals (the control group) contained no reticulocytes and almost no leukocytes; the platelet count was eight times lower than in intact rats (Table 16). One rat from the control group died as early as the beginning of day 3 of the experiment.

Rats treated with the RNA preparations (experimental groups) had a different peripheral blood pattern. In the rats injected with the preparation from the bone marrow of intact animals (RNAbm-1), reticulocytes were found in peripheral blood, and the leukocyte and platelet counts were, respectively, three times and 18.5% higher than in the control animals as early as the third day of the experiment. The shifts towards recovery were even greater in the rats treated with the RNA preparation from the bone marrow of anematized rats (RNAbm-2); their reticulocyte and leukocyte counts were two times higher, and the platelet count was 12.5% higher, than in the group treated with RNAbm-1. Thus, we may conclude that the RNAbm preparations caused partial restoration of erythropoiesis as early as the third day after irradiation, because appearance of peripheral blood reticulocytes in bone marrow damage with γ-radiation is the earliest and most reliable sign of hematopoiesis recovery [Internal Diseases: Military Field Therapeutics. Ed. by Rakov A. L., Sosyukin A. E. St. Petersburg: Foliant, 2003, 253 pp.].

This high rate of restoration of the peripheral components of the erythroid, myeloid, and megakaryocytic hematopoietic lineages was retained at later time points (on days 5 and 7 of the experiment). The reticulocyte count in the blood of the animals treated with the RNA preparations was substantially higher than in the control animals, the increase in this parameter being greater in the rats treated with RNAbm-2 than in those treated with RNAbm-1. In the control animals, reticulocytes did not appear in the peripheral blood until day 7. The leukocyte count of the animals treated with RNAbm-2 was higher compared to those treated with RNAbm-1 by a factor of 3 on day 5 and by a factor of 2.4 on day 7. The platelet count in the experimental groups on those days was almost two times higher than the control level. Note that, in addition to the improved peripheral blood parameters, a considerably improved animals' general condition was observed in the experimental groups. While the control rats did not exhibit any motor activity, exploratory behavior, or grooming until the seventh day of the experiment, the behavior of the experimental rats did not differ from that of intact rats in any respect as early as the third day after irradiation.

On the seventh day after irradiation, the experimental animals were additionally injected with the same RNA preparation (RNAbm-1 or RNAbm-2, in different groups) at a dose of 20 μg/100 g body weight.

By the tenth day of the experiment, one more rat had died in the control group; so, the mortality in this group became 40%. In the experimental groups, the erythrocyte count of the peripheral blood became significantly increased compared to the control value at this time point, against the background of reticulocyte, leukocyte, and platelet counts that were already higher than in the control group.

Having obtained evidence that the bone marrow RNA preparations considerably facilitated the restoration of the peripheral blood cell counts in sub-lethally irradiated animals, we assumed that RNA preparations from thymic lymphoid cells could support and enhance the effect on hematopoiesis recovery. Earlier, other researchers showed that screening of the thymus against radiation during acute irradiation of animals or administration of thymosin on the first days after the irradiation stimulated the regeneration of lymphoid tissues and hematopoietic organs [Moskalev, Y. L Long-term effects of ionizing radiation. M.: Meditsina. 1991. 464 pp.]. We attempted to reveal the possible additional stimulatory effect of RNA preparations from thymic lymphoid cells on hematopoiesis. For this purpose, on day 14 after sub-lethal irradiation, we injected the RNA preparations derived from the thymus of intact (RNAt-1) and anematized (RNAt-1) rats (30 μg/100 g body weight) to the rats that had been treated with RNAbm-1 and RNAbm-2, respectively. Hereinafter, this sequential administration of two preparations is denoted by their abbreviations separated by a comma: RNAbm-1, RNAt-1 and RNAbm-2, RNAt-2, respectively.

On day 15 after the start of the experiment (24 h after the additional injection of the thymic RNA preparations), the blood reticulocyte counts in the animals injected with the RNA preparations from the lymphoid organs of intact and anematized rats were, respectively, 3.1 and 4.7 times higher than the control level. The latter value did not differ significantly from the background one. Thus, the peripheral blood reticulocyte count of the rats treated with the RNA preparations from anematized animals reached the normal level by day 15 after γ-irradiation. The leukocyte and platelet counts also increased in this experimental group; they became, respectively, 3.7 and 2.1 times higher than in the control rats.

After that, the rate of restoration of the peripheral blood cell counts in the rats treated with RNA preparations from bone marrow and thymic lymphoid cells of anematized rats was higher than in the animals treated with the RNA preparations from lymphoid organs of intact rats until day 31 of observation.

By day 40 of the experiment, the reticulocyte count reached the normal level in all irradiated rats (in both control and experimental groups); the erythrocyte count was also equal to the background level. The leukocyte count did not reach the initial level, but it was within the species-specific normal range. The platelet count in the control rats and rats treated with the RNA preparations from lymphoid organs of intact rats was somewhat lower than the initial value; in the rats treated with RNA from lymphoid organs of anematized animals, it significantly exceeded the initial value.

TABLE 16
Effects of total RNA preparations derived from bone marrow (RNAbm) and thymic
(RNAt) lymphoid cells of intact and anematized animals on the blood cell counts
of rats subjected to acute γ-irradiation at the sub-lethal dose of 6 Gy

| | Parameterr | | | |
|---|---|---|---|---|
| | Reticulocytes | Erythrocytes | Leukocytes | Platelets |
| Group | (‰) | (×10¹²/l) | (×10⁹/l) | (×10⁹/l) |
| Background | 35.1 ± 1.4 | 6.9 ± 0.2 | 8.1 ± 0.1 | 395.1 ± 6.2 |
| (before irradiation) | | | | |

Day 3 after the first RNAbm injection

| | | | | |
|---|---|---|---|---|
| Control (irradiation) | 0^{□} | 5.6 ± 0.1^{□} | 0.03 ± 0.03^{□} | 54.3 ± 2.3^{□} |
| RNAbm-1 | 3.6 ± 0.5*^{□} | 5.7 ± 0.1^{□} | 0.1 ± 0.03^{□} | 64.4 ± 1.9*^{□} |
| RNAbm-2 | 7.0 ± 0.9*^{▪□} | 5.7 ± 0.03^{□} | 0.2 ± 0.02*^{□} | 72.8 ± 2.7*^{▪□} |

Day 5 after the first RNAbm injection

| | | | | |
|---|---|---|---|---|
| Control | 0^{□} | 5.6 ± 0.1^{□} | 0.1 ± 0.05^{□} | 51.3 ± 5.2^{□} |
| RNAbm-1 | 10.8 ± 1.0*^{□▴} | 5.8 ± 0.1^{□} | 0.2 ± 0.04^{□} | 80.0 ± 1.5*^{□▴} |
| RNAbm-2 | 16.4 ± 0.9*^{▪□▴} | 5.8 ± 0.1^{□} | 0.6 ± 0.1*^{▪□▴} | 94.4 ± 2.1*^{▪□▴} |

Day 7 after the first RNAbm injection

| | | | | |
|---|---|---|---|---|
| Control | 1.8 ± 0.5^{□▴} | 5.8 ± 0.2^{□} | 0.1 ± 0.04^{□} | 83.3 ± 2.7^{□▴} |
| RNAbm-1 | 15.0 ± 1.2*^{□▴} | 6.0 ± 0.1^{□} | 0.5 ± 0.1*^{□▴} | 139.0 ± 3.9*^{□▴} |
| RNAbm-2 | 21.8 ± 1.1*^{▪□▴} | 5.9 ± 0.1^{□} | 1.2 ± 0.1*^{▪□▴} | 182.2 ± 4.9*^{▪□▴} |

Day 10 after the first RNAbm injection (day 3 after the second RNAbm injection)

| | | | | |
|---|---|---|---|---|
| Control | 5.0 ± 0.6^{□} | 5.4 ± 0.3^{□} | 0.2 ± 0.03^{□} | 85.3 ± 4.8^{□} |
| RNAbm-1 | 17.6 ± 0.8*^{□} | 6.0 ± 0.1*^{□} | 1.5 ± 0.1*^{□▴} | 150.6 ± 5.7*^{□} |
| RNAbm-2 | 25.4 ± 0.7*^{▪□} | 6.1 ± 0.1*^{□} | 2.0 ± 0.1*^{▪□▴} | 204.2 ± 3.4*^{▪□} |

Day 15 after the first RNAbm injection (day 8 after the second RNAbm injection;
day 1 after additional RNAt injection)

| | | | | |
|---|---|---|---|---|
| Control | 6.3 ± 0.3^{□} | 5.6 ± 0.1^{□} | 0.9 ± 0.2^{□} | 101.3 ± 5.8^{□} |
| RNAbm-1 | 19.8 ± 1.1*^{□} | 5.8 ± 0.1^{□} | 2.0 ± 0.1*^{□▴} | 175.6 ± 11.0*^{□} |
| RNAbm-2 | 29.8 ± 1.5*^{▪} | 6.0 ± 0.1*^{□} | 3.3 ± 0.1*^{▪□▴} | 213.4 ± 6.9*^{▪□} |

Day 20 after the first RNAbm injection (day 13 after the second RNAbm injection;
day 6 after additional RNAt injection)

| | | | | |
|---|---|---|---|---|
| Control | 8.3 ± 0.9^{□} | 5.4 ± 0.1^{□} | 1.2 ± 0.1^{□} | 94.3 ± 3.5^{□} |
| RNAbm-1, RNAt-1 | 29.6 ± 1.5*^{▴} | 6.0 ± 0.1*^{□} | 3.0 ± 0.1*^{□▴} | 159.6 ± 9.7*^{□} |
| RNAbm-2, RNAt-2 | 38.2 ± 1.3*^{▪▴} | 6.4 ± 0.1*^{▴} | 4.2 ± 0.1*^{▪□▴} | 345.2 ± 9.7*^{▪□▴} |

Day 25 after the first RNAbm injection (day 18 after the second RNAbm injection;
day 11 after additional RNAt injection)

| | | | | |
|---|---|---|---|---|
| Control | 13.3 ± 1.2^{□} | 5.7 ± 0.1^{□} | 2.2 ± 0.2^{□▴} | 114.3 ± 2.3^{□} |
| RNAbm-1, RNAt-1 | 31.2 ± 1.9* | 6.4 ± 0.2*^{▴} | 4.5 ± 0.2*^{□▴} | 163.2 ± 8.1*^{□} |
| RNAbm-2, RNAt-2 | 39.0 ± 1.5*^{▪} | 7.4 ± 0.2*^{▪▴} | 6.2 ± 0.2*^{▪□▴} | 416.4 ± 7.0*^{▪▴} |

Day 31 after the first RNAbm injection (day 24 after the second RNAbm injection;
day 17 after additional RNAt injection)

| | | | | |
|---|---|---|---|---|
| Control | 21.3 ± 2.6^{□} | 5.9 ± 0.2^{□} | 4.2 ± 0.2^{□▴} | 181.7 ± 6.9^{□▴} |
| RNAbm-1, RNAt-1 | 30.2 ± 1.8 | 7.0 ± 0.2*^{▴} | 6.0 ± 0.2*^{□▴} | 199.2 ± 6.2^{□▴} |
| RNAbm-2, RNAt-2 | 38.0 ± 3.0*^{▪} | 7.9 ± 0.2*^{▪□▴} | 6.9 ± 0.3*^{▪□} | 421.4 ± 5.5*^{▪} |

Day 40 after the first RNAbm injection (day 33 after the second RNAbm injection;
day 26 after additional RNAt injection)

| | | | | |
|---|---|---|---|---|
| Control | 32.3 ± 2.7 | 6.9 ± 0.2 | 7.1 ± 0.3^{□▴} | 364.7 ± 25.4^{▴} |
| RNAbm-1, RNAt-1 | 31.8 ± 2.2 | 7.0 ± 0.2 | 7.6 ± 0.2^{□▴} | 355.2 ± 11.0^{□▴} |
| RNAbm-2, RNAt-2 | 37.2 ± 2.5 | 7.6 ± 0.2*^{▪} | 7.3 ± 0.3^{□} | 430.4 ± 10.7^{▪□} |

Notes:
*Significant difference from the control group;
^{▪}significant difference from the animals treated with RNA-1 preparations;
^{□}significant difference from the background;
^{▴}significant difference from the preceding time point (p < 0.05).

When analyzing the bone marrow cell composition on day 45 after irradiation, we found that, notwithstanding the almost complete normalization of the peripheral blood cell counts, hematopoiesis in control rats differed from that in intact rats in that blasts and juvenile blood cells of both white cell and red cell lineages were entirely absent, and intermediate forms were considerably fewer (Table 17). Most bone marrow hematopoietic cells of the control rats were differentiated cells, namely, banded and segmented neutrophils, eosinophils, and oxyphilic erythroblasts. The hematopoietic cell composition of the bone marrow of the rats treated with RNA preparations from lymphoid organs of intact animals differed from the normal one only in a 1.4-fold smaller proportion of promyelocytes. The RNA preparations of the disclosure isolated from lymphoid organs of anematized rats, injected to sub-lethally irradiated rats, stimulated hematopoiesis, especially the erythroid lineage, more strongly.

TABLE 17
Percentages of hematopoietic cells in the bone marrow of sub-lethally
irradiated rats treated with the RNA-1 and RNA-2 preparations in
the period of restoration of peripheral blood cell counts

| | Group | | | |
|---|---|---|---|---|
| | | Control | | |
| Parameter | Intact rats | (without treatment) | RNA-1 | RNA-2 |
| Myeloblasts | 2.0 ± 0.3 | 0^{□} | 1.5 ± 0.5* | 1.8 ± 0.4* |
| Promyelocytes | 2.6 ± 0.3 | 0.7 ± 0.3^{□} | 1.8 ± 0.2*^{□} | 2.4 ± 0.2* |
| Myelocytes | 4.4 ± 0.2 | 2.3 ± 0.3^{□} | 3.7 ± 0.2* | 4.6 ± 0.2*^{▪} |
| Metamyelocytes | 13.2 ± 0.4 | 7.0 ± 0.6^{□} | 11.0 ± 0.4* | 13.0 ± 0.3*^{▪} |
| Banded neutrophils | 17.2 ± 0.4 | 27.7 ± 1.2^{□} | 16.6 ± 0.5* | 16.2 ± 0.4* |
| Segmented neutrophils | 16.2 ± 0.9 | 29.0 ± 0.6^{□} | 14.6 ± 0.5* | 14.8 ± 0.4* |
| Eosinophils | 4.6 ± 0.2 | 7.0 ± 1.0^{□} | 4.8 ± 0.4* | 4.4 ± 0.2* |
| Basophils | 0.4 ± 0.2 | 2.3 ± 0.3^{□} | 0.4 ± 0.2* | 0.7 ± 0.2* |
| Lymphoid cells | 10.2 ± 0.6 | 0.3 ± 0.3^{□} | 10.6 ± 0.7* | 12.2 ± 0.9* |
| Proerythroblasts | 0.4 ± 0.2 | 0^{□} | 0.6 ± 0.2* | 1.2 ± 0.2*^{▪□} |
| Basophilic | 6.2 ± 0.7 | 0.3 ± 0.3^{□} | 4.2 ± 0.6* | 7.2 ± 0.4*^{▪} |
| erythroblasts | | | | |
| Polychromatophilic | 12.8 ± 0.6 | 4.0 ± 0.6^{□} | 10.6 ± 0.5* | 14.0 ± 0.3*^{▪} |
| erythroblasts | | | | |
| Oxyphilic erythroblasts | 12.0 ± 1.1 | 17.7 ± 0.3^{□} | 12.2 ± 0.3* | 11.8 ± 0.7* |
| Megakaryocytes | 1.1 ± 0.1 | 0.2 ± 0.1^{□} | 0.6 ± 0.04* | 1.0 ± 0.1*^{▪} |

Notes:
*Significant difference from the control group;
^{▪}significant difference from the animals treated with RNA-1 preparations;
^{□}significant difference from the intact animals (p < 0.05).

In order to study the proliferation, differentiation, and maturation of erythroid cells under the conditions of our experiment in more detail, we analyzed the quantitative and qualitative compositions of bone marrow EIs of the control and experimental animals (Table 18). It was found that the proportion of proliferating EIs and the number of mature (class 3) EIs in the bone marrow were significantly increased 45 days after irradiation. It is noteworthy that the RNA preparations from lymphoid organs of anematized and intact rats differed from each other in the pattern of the effect on erythropoiesis in EIs. The RNA preparations from lymphoid organs of intact rats stimulated erythropoiesis only through the formation of new EIs, whereas those from lymphoid organs of anematized rats induced intense EI reconstruction as well. This stimulation of the interaction of CFU-e with both free macrophages and those that had previously been involved in erythropoiesis in the rats treated with the latter preparations led to a significant increase in the absolute number of EIs in the bone marrow of these animals.

TABLE 18
The quantitative and qualitative compositions of EIs of the bone marrow of sub-
lethally irradiated rats sequentially treated with either RNAbm-1, RNAt-1 or
RNAbm-2, RNAt-2 in the period of restoration of peripheral blood cell counts

| | Group | | | |
|---|---|---|---|---|
| Parameter | Intact rats | Control | RNAbm-1, RNAt-1 | RNAbm-2, RNAt-2 |
| EIs, abs. number | 254.6 ± 4.4 | 251.7 ± 8.6 | 257.2 ± 5.2 | 316.2 ± 3.9*^{▪□} |
| (10³/femoral bone) | | | | |
| Class 1 EIs, % | 5.4 ± 0.7 | 2.3 ± 0.3^{□} | 5.2 ± 0.5* | 8.0 ± 0.3*^{▪□} |
| Class 2 EIs, % | 8.2 ± 0.6 | 5.7 ± 0.3^{□} | 7.2 ± 0.4* | 9.2 ± 0.4*^{▪} |
| Class 3 EIs, % | 25.0 ± 0.8 | 19.0 ± 0.6^{□} | 23.6 ± 1.2* | 24.6 ± 0.9* |
| Inv. EIs, % | 50.2 ± 1.4 | 61.7 ± 0.7^{□} | 52.8 ± 1.1* | 40.8 ± 1.1*^{▪□} |
| Rec. EIs, % | 11.2 ± 0.7 | 11.3 ± 0.7 | 12.0 ± 0.5 | 17.4 ± 0.9*^{▪□} |

Notes:
*Significant difference from the control group;
^{▪}significant difference from the animals treated with RNAbm-1, RNAt-1;
^{□}significant difference from the intact animals (p < 0.05).

Changes in the parameters of EI development rate (Table 19) also demonstrated a strong erythropoiesis-stimulating effect of the RNA preparations derived from lymphoid organs of anematized rats. The calculated indices indicate that the preparations according to the invention significantly increased the intensity of CFU-e involvement in erythropoiesis and the rate of erythroid cell maturation in EIs, as well as stimulated the reinvolvement of macrophages in the EI formation, thereby promoting erythropoiesis reconstruction in the bone marrow.

TABLE 19
Calculated indices of erythropoiesis in bone marrow EIs of sub-lethally
irradiated rats sequentially treated with the RNAbm and RNAt preparations
in the period of restoration of peripheral blood cell counts

| | Parameter | | |
|---|---|---|---|
| | CFU-e | | Macrophage |
| | involvement in | Erythroid cell | reinvolvement |
| Group | differentiation | maturation in EIs | in erythropoiesis |
| Intact rats | 16.6 ± 0.9 | 0.33 ± 0.02 | 0.23 ± 0.02 |
| Control | 13.7 ± 0.3^{□} | 0.27 ± 0.04 | 0.16 ± 0.01^{□} |
| RNAbm-1, RNAt-1 | 17.6 ± 0.7 | 0.32 ± 0.01 | 0.23 ± 0.02* |
| RNAbm-2, RNAt-2 | 25.4 ± 1.2*^{▪□} | 0.53 ± 0.02*^{▪□} | 0.43 ± 0.03*^{▪□} |

Notes:
*Significant difference from the control group;
^{▪}significant difference from the animals treated with the RNA preparations from intact rats (RNA-1);
^{□}significant difference from the intact animals (p < 0.05).

We found that the more rapid and complete restoration of peripheral blood cell counts in the irradiated rats treated with the stimulatory RNA preparations from lymphoid cells was determined not only by higher doses of the preparations (compared to the dose of 15 μg/100 g body weight in Examples 1-5), but also by the specific characteristics of the cell population from which the total RNA preparation of the disclosure was derived. We noticed an especially strong effect of the RNA preparation from the bone marrow of anematized rats. This may have been explained by the presence of stem cells in the bone marrow; however, further studies were required to test this assumption. Their results are described in the following sections.

The data presented in this section clearly show that under the influence of the same stimulus activation of lymphoid cells occurs simultaneously and unidirectionally. It has been demonstrated that the effects of the RNA preparations of the disclosure isolated from them are mutually interchangeable and quite compatible.

Note that the total RNA preparations from lymphoid organs of intact animals also possess stimulatory activity, although this activity is weaker. It should be also emphasized that a complete restoration of hematopoiesis, i.e., restoration of all hematopoietic lineages in our experiments was reached within a little longer than a month.

These results also suggested that it could be reasonable to use combinations of total RNA preparations derived from different organs. One of the possible ways to test this suggestion was to study the restoration of hematopoietic tissue after chronic benzene intoxication (model IV) in rats treated with the total RNA preparation according to the invention isolated from peripheral blood lymphocytes of healthy donors (RNApbl-1). Indeed, the homeostasis control mechanisms must ensure that peripheral blood contains the optimal combination of lymphocytes originating from different lymphoid organs.

The results of this experiment are of special interest.

### Example 7

### Effects of the Total RNA Preparation from Peripheral Blood Lymphocytes of Healthy Donors on the Restoration of Peripheral Blood Cell Counts and Bone Marrow Hematopoiesis in Rats with Benzene-Induced Chronic Hypoplastic Anemia

The specificity of this experiment was that the total RNA preparation was isolated from a pure fraction of unstimulated lymphocytes of the peripheral blood of healthy human donors (hRNApbl-1). The results have allowed us to make two interesting conclusions.

First, the activity of the total RNA preparation from human peripheral blood lymphocytes has proved to be as high as that of the total RNA preparation derived from the total (unseparated) lymphoid cell population.

Second, the results of the experiment have unambiguously confirmed that the RNA preparation activity is not species-specific, so that xenogeneic total RNA preparations can be efficiently used.

The experiment was carried out on 10 female outbred white rats weighing 120-140 g. Hypoplastic anemia was induced in the animals by four benzene injections as described in the “Materials and Methods” (see “Model (IV) of benzene-induced chronic hypoplastic anemia”). Thirty-five days after the last benzene injection, the peripheral blood cell count in the animals was considerably decreased, which indicated a hypoplasia of all hematopoietic lineages in the bone marrow. After estimation of the peripheral blood parameters, we divided the animals into two groups. Experimental rats were each injected once with the hRNApbl-1 preparation derived from lymphocytes of healthy donors at a dose of 30 μg/100 g body weight. Control rats were each the same way injected with 0.1 ml of 0.9% NaCl.

The results showed that a single injection of the hRNApbl-1 preparation led to a complete normalization of hematopoiesis in the rats with benzene-induced anemia within 16-21 days after injection (Table 20).

The peripheral blood reticulocyte count in the experimental rats was significantly higher than in the control group five days after the injection of the hRNApbl-1 preparation (Table 20). By day 10 after the injection, the leukocyte count began increasing as well. Twenty-one day after the hRNApbl-1 injection, even an increase in the erythrocyte count was detected. Note that a particularly dramatic increase in the peripheral blood reticulocyte, leukocyte, and platelet counts was observed in the period between days 16 and 30: every five days, these counts proved to be significantly higher than at the preceding time point.

TABLE 20
Effect of the total RNA preparation derived from human peripheral
blood lymphocytes (hRNApbl-1) on the peripheral blood cell
counts in rats with benzene-induced anemia (model IV)

| | Parameter | | | |
|---|---|---|---|---|
| | Reticulocytes | Erythrocytes | Leukocytes | Platelets |
| Group | (‰) | (×10¹²/l) | (×10⁹/l) | (×10⁹/l) |
| Background | 6.4 ± 0.3 | 5.5 ± 0.2 | 2.5 ± 0.1 | 87.7 ± 2.8 |
| (benzene- | | | | |
| induced anemia) | | | | |

Day 5 after the hRNApbl-1 injection

| | | | | |
|---|---|---|---|---|
| Control | 6.6 ± 0.5 | 5.5 ± 0.2 | 2.5 ± 0.05 | 86.8 ± 2.2 |
| hRNApbl-1 | 10.8 ± 0.6* | 5.8 ± 0.1 | 2.8 ± 0.05 | 91.0 ± 1.7 |

Day 10 after the hRNApbl-1 injection

| | | | | |
|---|---|---|---|---|
| Control | 6.8 ± 0.4 | 5.7 ± 0.2 | 2.7 ± 0.07 | 90.0 ± 3.2 |
| hRNApbl-1 | 13.2 ± 0.6* | 5.9 ± 0.1 | 3.2 ± 0.07* | 98.8 ± 1.4 |

Day 16 after the hRNApbl-1 injection

| | | | | |
|---|---|---|---|---|
| Control | 7.6 ± 0.5 | 5.6 ± 0.2 | 2.7 ± 0.1 | 91.6 ± 4.0 |
| hRNApbl-1 | 12.2 ± 0.7* | 6.0 ± 0.1 | 3.2 ± 0.08* | 113.8 ± 2.6*^{▴} |

Day 21 after the hRNApbl-1 injection

| | | | | |
|---|---|---|---|---|
| Control | 11.0 ± 0.6 | 5.5 ± 0.1 | 2.8 ± 0.09 | 109.2 ± 4.6 |
| hRNApbl-1 | 17.0 ± 1.3*^{▴} | 6.2 ± 0.1* | 3.5 ± 0.1* | 125.2 ± 3.3*^{▴} |

Day 25 after the hRNApbl-1 injection

| | | | | |
|---|---|---|---|---|
| Control | 13.4 ± 1.1 | 5.6 ± 0.1 | 2.9 ± 0.08 | 109.0 ± 3.5 |
| hRNApbl-1 | 21.6 ± 1.4*^{▴} | 6.1 ± 0.1* | 4.2 ± 0.2* | 148.8 ± 4.1*^{▴} |

Day 30 after the hRNApbl-1 injection

| | | | | |
|---|---|---|---|---|
| Control | 14.2 ± 1.4 | 6.5 ± 0.2 | 2.8 ± 0.2 | 117.0 ± 5.4 |
| hRNApbl-1 | 29.8 ± 1.7*^{▴} | 7.6 ± 0.3* | 4.7 ± 0.3* | 201.2 ± 12.6*^{▴} |

Day 36 after the hRNApbl-1 injection

| | | | | |
|---|---|---|---|---|
| Control | 12.2 ± 1.2 | 6.0 ± 0.2 | 2.9 ± 0.2 | 107.8 ± 5.5 |
| hRNApbl-1 | 28.4 ± 2.3* | 8.1 ± 0.2* | 4.4 ± 0.3* | 223.8 ± 5.3* |

Day 45 after the hRNApbl-1 injection

| | | | | |
|---|---|---|---|---|
| Control | 15.8 ± 1.7 | 5.8 ± 0.2 | 3.3 ± 0.2 | 129.6 ± 5.1 |
| hRNApbl-1 | 29.0 ± 2.4* | 7.6 ± 0.1* | 5.0 ± 0.3* | 327.8 ± 14.2* |

Day 57 after the hRNApbl-1 injection

| | | | | |
|---|---|---|---|---|
| Control | 20.8 ± 1.7 | 6.4 ± 0.1 | 5.4 ± 0.2 | 240.4 ± 8.9 |
| hRNApbl-1 | 30.2 ± 1.6* | 7.7 ± 0.1* | 8.5 ± 0.3* | 394.0 ± 11.9* |

Day 70 after the hRNApbl-1 injection

| | | | | |
|---|---|---|---|---|
| Control | 21.8 ± 1.3 | 6.6 ± 0.1 | 6.8 ± 0.1 | 223.6 ± 5.4 |
| hRNApbl-1 | 30.8 ± 1.4* | 7.6 ± 0.1* | 8.2 ± 0.2* | 401.6 ± 7.4* |

Notes:
*Significant difference from the control group;
^{▴}significant difference from the preceding time point (p < 0.05).

The effectiveness of the RNA preparation derived from peripheral blood lymphocytes of healthy donors eliminates many potential problems with future production of a commercial erythropoiesis-stimulating preparation.

However, our experiments described in the preceding examples have demonstrated that the RNA preparations derived from stimulated lymphoid cells are more effective than those from unstimulated cells (isolated from intact animals). Therefore, we assume that, in the cases when more rapid and intense stimulation of hematopoiesis is necessary, it would be reasonable to use peripheral blood lymphocytes from donors living in highland regions, because their hematopoiesis is naturally stimulated. In addition, allogeneic variants of total RNA preparations with even higher stimulatory and inhibitory activities could be isolated from the T helper and T suppressor cell fractions separated by means of a cell sorter.

It is known that the activity of T suppressor cells is considerably less tissue-specific than that of T helper cells, inhibiting proliferation not only in their original tissue, but also in other ones. This further extends the possibility of using regulatory lymphoid cells and the total RNA preparations of the disclosure that are derived from them.

The above results indicating a real possibility of effective treatment for anemia raised a question of whether, and how much, the preparations of the disclosure would be effective when administered via other, less invasive or noninvasive routes, including the intranasal one. Our comparative estimation of different routes of administration is described in Example 8.

### Example 8

### Effectiveness of the Total RNA Preparations as Dependent on the Route of Administration

In order to compare different routes of administration of the total RNA preparations in terms of treatment effectiveness, we carried out a series of experiments using model (IV) of benzene-induced chronic hypoplastic anemia. All experimental groups of rats were treated with the stimulatory RNAs-2 preparation.

Thirty male rats weighing 190-210 g were used in the experiment.

The following protocol was used for subcutaneous (SC), intramuscular (IM), intraperitoneal (IP), and intravenous (IV) administrations: the first administration at a dose of 30 μg/100 g body weight and the second administration (seven days later) at a dose of 10 μg/100 g body weight.

For intranasal (IN) administration, we used a different protocol: three daily administrations in drops to both nostrils for three consecutive days at doses of 10 μg/100 g body weight and the fourth administration at the same dose seven days after the third one.

TABLE 21
Changes in the peripheral blood cell counts in rats with benzene-induced anemia
(model IV) treated with the RNAs-2 preparation administered via different routes

| | Parameter | | | |
|---|---|---|---|---|
| | Reticulocytes | Erythrocytes | Leukocytes | Platelets |
| Group | (‰) | (×10¹²/l) | (×10⁹/l) | (×10⁹/l) |
| Background | 32.3 ± 0.8 | 6.9 ± 0.1 | 8.6 ± 0.1 | 395.4 ± 5.6 |
| (intact rats) | | | | |

7 days after the first RNAs-2 injection

| | | | | |
|---|---|---|---|---|
| Control (benzene- | 6.6 ± 0.5 | 5.6 ± 0.1 | 2.4 ± 0.04 | 84.0 ± 1.8 |
| induced anemia) | | | | |
| SC administration | 6.0 ± 0.4 | 5.7 ± 0.1 | 2.4 ± 0.07 | 86.6 ± 2.3 |
| IM administration | 8.4 ± 0.5* | 5.7 ± 0.1 | 2.5 ± 0.07 | 86.8 ± 2.7 |
| IP administration | 13.0 ± 0.9* | 5.8 ± 0.1 | 2.9 ± 0.09* | 106.2 ± 2.2* |
| IV administration | 15.2 ± 0.9* | 5.5 ± 0.1 | 3.9 ± 0.1* | 138.6 ± 2.1* |
| IN administration | 11.0 ± 0.7* | 5.4 ± 0.2 | 3.4 ± 0.1* | 101.0 ± 2.2* |

14 days after the first RNAs-2 injection

| | | | | |
|---|---|---|---|---|
| Control (benzene- | 7.2 ± 0.4 | 5.6 ± 0.1 | 2.4 ± 0.07 | 90.6 ± 3.5 |
| induced anemia) | | | | |
| SC administration | 5.8 ± 0.7 | 5.9 ± 0.1 | 2.4 ± 0.05 | 93.8 ± 3.0 |
| IM administration | 8.6 ± 0.9 | 5.8 ± 0.1 | 2.5 ± 0.06 | 104.8 ± 2.7* |
| IP administration | 18.6 ± 1.1* | 5.9 ± 0.1 | 3.0 ± 0.09* | 126.6 ± 3.0* |
| IV administration | 19.0 ± 1.1* | 5.9 ± 0.2 | 3.6 ± 0.1* | 145.6 ± 2.6* |
| IN administration | 14.4 ± 0.4* | 5.8 ± 0.1 | 3.4 ± 0.1* | 130.8 ± 3.6* |

20 days after the first RNAs-2 injection

| | | | | |
|---|---|---|---|---|
| Control (benzene- | 7.2 ± 0.6 | 5.5 ± 0.03 | 2.8 ± 0.09 | 105.2 ± 3.2 |
| induced anemia) | | | | |
| SC administration | 8.0 ± 0.5 | 5.8 ± 0.1* | 2.6 ± 0.05 | 110.0 ± 2.2 |
| IM administration | 15.0 ± 1.4* | 6.0 ± 0.1* | 3.3 ± 0.07* | 127.4 ± 2.6* |
| IP administration | 27.0 ± 1.6* | 6.8 ± 0.2* | 3.6 ± 0.09* | 168.4 ± 4.1* |
| IV administration | 28.7 ± 0.9* | 7.0 ± 0.2* | 4.2 ± 0.08* | 182.4 ± 3.3* |
| IN administration | 21.8 ± 0.9* | 6.9 ± 0.1* | 3.8 ± 0.1* | 157.8 ± 4.0* |

Notes:
*Significant difference from the control group (Mann-Whitney and Kruskal-Wallis tests, p < 0.05).

In addition to the statistical treatment of the data described in the “Materials and Methods,” we used cluster analysis to determine the most effective routes of RNAs-2 administration. As a result, the groups of animals were divided into two clusters. The first cluster comprised the control group and the groups with subcutaneous and intramuscular injections of the preparation. The second cluster comprised the experimental groups with intravenous, intraperitoneal, and intranasal administrations of RNAs-2. This suggests that, despite the differences obtained in this experiment, the intraperitoneal and intranasal administrations of the RNAs-2 preparations are no less effective (and, hence, promising for further use) than its intravenous injection. Therefore, we also used the intranasal route for administering other preparations according to the invention. For example, in treating experimental diabetes mellitus, intranasal administration, along with intravenous and intraperitoneal ones, proved to be not only acceptable, but exceptionally effective.

In this connection, there are grounds to believe that rectal administration of the preparations according to the invention will also prove no less effective.

On the other hand, the results of our experiments with subcutaneous and intramuscular administrations only mean that these routes are unsuitable for obtaining a therapeutic effect at the whole-body level, or that higher doses of the preparations are required in this case. However, as will be evident from Example 13, even external (local) use of the preparations of the disclosure may be sufficiently effective in other cases.

According to the basic concept of our invention, the system of immunogenesis as a general regulatory system of the body should have modulatory effects on not only lymphoid tissues and not only hematopoietic cells, but also cells of other histotypes. In addition, according to other authors, RNA preparations isolated from cells of a given organ have favorable effects on cells of the same organ or tissue of other organisms.

Thus, our data and accumulated previous knowledge directly lead us to exploring the possibility of the treatment of diseases whose pathogenesis involves disturbances in the system of lymphoid cells. In this connection, of special interest are our data on the use of the total RNA preparation isolated from human peripheral blood lymphocytes (see Example 7). This preparation, which does not require matching of blood group or histocompatibility antigens, and which can be administered intranasally, can be used in medical practice in the near future.

### Example 9

### Effects of the Total RNA Preparations Derived from Spleen Lymphoid Cells on the Condition of C57BL/RsJYLepr^{db/+} Mice with Type 2 Diabetes Mellitus

In the first preliminary experiments (quantitative data not shown), we studied the effect of a single injection of the RNAs-1, RNAs-2, and RNAs-3 preparations derived from rat spleen lymphoid cells on the condition of C57BL/KsJYLepr^{db/+} mice, which have been demonstrated to be an adequate experimental model for studying type 2 diabetes mellitus [Stepanova O. I., Karkischenko V. N., Baranova O. V., Galahova T. V., Semenov X. X., Beskova T. B., Stepanova E. A., Zakir′yanov A. R., Onischenko N. A. Mutant C57BL/KsJYLepr^{db/+} mice as a genetic model of type 2 diabetes mellitus. Bull. Exp. Biol. Med., 2007, vol. 144, issue 6, pp. 813-816]. The results of our preliminary study suggested favorable but qualitatively different effects of the RNA preparations according to the invention on the general condition of the animals and their blood glucose levels.

Specifically, favorable changes in the blood glucose level was observed in about 40% of a total of 28 mice as early as day 6 after a single intraperitoneal injection of the RNAs-1 or RNAs-2 preparation. In two mice with an initial glucose level >33, this parameter decreased by 35.2 and 25.1%, respectively, in response to a single RNAs-2 injection, after which it steadily decreased for 49 days (until the animals were euthanized) in one of them and for 35 days in the other one.

It is also noteworthy that a single intraperitoneal injection of RNAs-1 caused slow but complete healing of skin macerations in all animals that initially had them (8 out of 28 mice). Note that, according to literature data [Stepanova O. I. Bone marrow cell transplantation for correcting pathogenetic disturbances in type 2 diabetes mellitus. Cand. Sci. (Biol.) Dissertation. Moscow, 2009], 20-25% of db/db mice with diabetes mellitus develop skin maceration at the shoulder top at an age of 120-158 days; within the next 5-14 days, the maceration became a large, nonhealing wound and remained there until the animals died. Another important result of our experiments was that the wound-healing effect of a single RNAs-1 injection was prolonged (being observed for more than a month). After that, spots of skin maceration appeared in some animals, which correlated with fading of the effect of the single preparation dose, because the blood glucose level rose at that stage. The treatment with RNAs-1 or RNAs-2 normalized the body weight and decreased diuresis in most animals; the mice began to consume less water and food.

A histological study was performed two months after the start of treatment with the RNA preparations according to the invention, and the results were compared with histological data on control db/db diabetic mice of the same strain and the same age (four to six months) [Stepanova O. I. Bone marrow cell transplantation for correcting pathogenetic disturbances in type 2 diabetes mellitus. Cand. Sci. (Biol.) Dissertation. Moscow, 2009]. The histological study of the pancreas of the untreated four- to six-month-old C57BL/KsJYLepr^{db/+} mice serving for modeling type 2 diabetes mellitus showed signs of manifest periductal and intralobular sclerosis, atrophy of the gland parenchyma, and intra- and perilobular lipomatosis. Very small atrophied pancreatic islets in the form of aggregations of small numbers of basophilic cells were observed between interlayers of connective and adipose tissues. The spleen of these mice underwent progressive hypoplasia. Signs of hypoplasia and atrophy were found in spleen lymphoid follicles. The area of lymphoid follicles in the spleen and the regional lymph node was more than two times smaller compared to control healthy mice.

Two months after the start of treatment with the RNA preparations of the disclosure, the number of pancreatic islets in the pancreas of the treated animals was practically normal; the islets were of medium size and regular oval or rounded shape, clearly outlined. All islets were cellular. The stromal vessels were filled with blood. In the spleen, signs of moderate lymphoid tissue hyperplasia and formation of sparse lymphoid follicles were observed, with a high blood filling of the red pulp. It is noteworthy that the treatment with the preparations of the disclosure also led to an increased blood filling of the liver and kidney tissues.

Thus, the results of preliminary studies on the experimental model of type 2 diabetes mellitus performed to date have clearly shown that the RNAs-1 preparation increases the regeneration capacity of not only the glandular epithelium of pancreas but also skin epithelium.

The authors of the patent RU 2400822 note that a drawback of genetic models of diabetes mellitus is that the disease develops in animals hereditarily predisposed to it; hence, the compensatory mechanisms and regeneration of pancreatic islet tissue in them are altered due to inherently abnormal responses of adaptive systems of the body. In contrast, the autoimmune model of diabetes mellitus obtained by combined administration of Freund's adjuvant and alloxan (patent RU 2400822) to healthy rats has made it possible to develop protocols for adequate treatment of this disease in animals without genetic defects of the immune system.

It should be added that, although the genetic model is the closest population model, it is not standardized and is characterized by large individual variations even in age-matched groups, which makes its use problematic in terms of experimental studies and statistical treatment of the results.

### Example 10

### Treatment of Rats with Experimental Alloxan-Induced Type 1 Diabetes Mellitus (Model (VI))

### Method 1.

When planning the experiments on treatment of alloxan-induced type 1 diabetes mellitus (DM), we took into consideration the finding of pathology of small blood vessels (microangiopathy) in the bone marrow of diabetic patients (medicalnewstoday.com, http:/www.medkurs.ru/news/39325.html). This, together with our own data indicating a high regulatory activity of the total RNA preparations from the bone marrow presented above, was an additional consideration in favor of using the RNAbm preparations in integrated treatment of alloxan-induced DM. In addition, we considered different types of RNA preparations from spleen lymphoid cells to be equally important components of the treatment. Finally, we derived a total RNA preparation from a homogenate of rat pancreas (RNAp) by the same method as the other preparations of the disclosure. This preparation was also used in the protocols of treatment of alloxan-induced DM. We proceeded from the assumption that normalization of all processes in the body affected by this pathology requires not only normalizing regulatory mechanisms with the use of RNA preparations from lymphoid cells of healthy animals, but also supporting the functional reserve of the recipient's pancreas. In addition, a number of publications related to prior art reported favorable effects of exogenous RNA isolated from different organs of the donor on the functions of the same organs of the recipient.

Experimental rats were divided into groups of five animals each. Control rats with experimental type 1 DM were intraperitoneally injected with the same volumes of 0.9% NaCl as the volumes of the preparations injected to experimental animals at the same time points.

A total of 60 rats were used in two series of experiments. In the first series, the effects of different preparations according to the invention (RNAbm-1, RNAbm-2, RNAs-1, RNAs-2, RNAt-3, and RNAp) and their combinations administered in different sequences were studied stage by stage in a total of 35 rats divided into six experimental groups and one control group of five animals each. In the experimental groups (a total of 30 rats), different modes and protocols of administration of the RNA preparations were tested, the preparations being administered at a dose of 15 μg/100 g body weight every seven days in each case.

In the second series of experiments, 25 rats were used, divided into four experimental groups and one control group of five animals each. In these experiments, we modified some aspects of the treatment protocols used in the first series by varying the intervals between the administrations of different preparations. In addition, we explored the possibility of simultaneous administration of the RNA preparations whose combinations, in the first series of experiments, proved to be optimal for completely normalizing the blood glucose level in the experimental animals. We also varied the doses and routes of administration of the optimal combinations of preparations of the disclosure.

Table 22 shows the data on three experimental groups where the treatment protocols proved to be optimal.

Data on the groups with the following treatment protocols are presented:

(1) Control (alloxan-induced DM).

(2) RNAbm-1, RNAs-1, and RNAp (separately; intraperitoneal administration).

(3) RNAbm-1+RNAs-1+RNAp (mixed; intraperitoneal administration).

(4) RNAbm-1+RNAs-1+RNAp (mixed; intranasal administration).

TABLE 22
Protocols of treatment of experimental type 1 diabetes mellitus
with RNA preparations in different combinations

| | Time | | |
|---|---|---|---|
| Group | Start of treatment | Day 7 | Day 14 |
| 1 | 0.9% NaCl | 0.9% NaCl | 0.9% NaCl |
| (control) | | | |
| 2 | RNAbm-1 | RNAs-1 | RNAp |
| (experiment) | (15 μg/100 g) | (15 μg/100 g) | (15 μg/100 g) |
| | intraperitoneally | intraperitoneally | intraperitoneally |
| 3 | RNAbm-1 + | RNAbm-1 + | RNAbm-1 + |
| (experiment) | RNAs-1 + RNAp | RNAs-1 + RNAp | RNAs-1 + RNAp |
| | (5 + 5 + 5) μg/ | (5 + 5 + 5) μg/ | (5 + 5 + 5) μg/ |
| | 100 g | 100 g | 100 g |
| | intraperitoneally | intraperitoneally | intraperitoneally |
| 4 | RNAbm-1 + | RNAbm-1 + | RNAbm-1 + |
| (experiment) | RNAs-1 + RNAp | RNAs-1 + RNAp | RNAs-1 + RNAp |
| | (5 + 5 + 5) μg/ | (5 + 5 + 5) μg/ | (5 + 5 + 5) μg/ |
| | 100 g | 100 g | 100 g |
| | intranasally | intranasally | intranasally |

Table 23 shows the results of DM treatment in the same three experimental groups where the optimal treatment protocols were used.

TABLE 23
Effects of different combinations of RNA preparations on the blood glucose level
(in millimoles per liter) in rats with experimental type 1 diabetes mellitus

| | Group | | | |
|---|---|---|---|---|
| | 1 | 2 | 3 | 4 |
| Time point | (control) | (experiment) | (experiment) | (experiment) |
| Background | 21.73 ± 0.46 | 21.76 ± 0.86 | 22.04 ± 0.44 | 21.64 ± 0.43 |
| Day 3 | 21.58 ± 0.64 | 11.98 ± 0.41* | 12.48 ± 0.52* | 13.46 ± 0.71* |
| Day 7 | 21.09 ± 0.31 | 11.40 ± 0.49* | 11.32 ± 0.65* | 11.34 ± 0.41* |
| Day 10 | 20.42 ± 0.41 | 8.90 ± 0.41* | 11.94 ± 0.57*^{▪} | 11.76 ± 0.69* |
| Day 14 | 18.87 ± 0.53 | 8.26 ± 0.24* | 9.28 ± 0.61* | 9.72 ± 0.47* |
| Day 17 | 18.72 ± 0.36 | 6.72 ± 0.21* | 9.46 ± 0.27*^{▪} | 9.71 ± 0.35* |
| Day 21 | 19.12 ± 0.41 | 5.86 ± 0.21* | 8.04 ± 0.40*^{▪} | 8.30 ± 0.36* |
| Day 24 | 18.53 ± 0.36 | 5.52 ± 0.18* | 6.78 ± 0.20*^{▪} | 7.18 ± 0.32* |
| Day 28 | 18.19 ± 0.46 | 5.56 ± 0.18* | 6.34 ± 0.22*^{▪} | 6.56 ± 0.19* |
| Day 42 | 18.68 ± 1.02 | 5.54 ± 0.15* | 5.16 ± 0.14* | 5.62 ± 0.12* |

Notes:
*Significant difference from the control group;
^{▪}significant differences between groups 2 and 3
The significance of differences was estimated using the Kruskal-Wallis, Mann-Whitney, and Wilcoxon tests. The differences were considered significant at a probability of type I error <0.05.

Another group of rats with alloxan diabetes was once i.p. administered simultaneously all three total RNA preparations of the disclosure, RNAbm-1+RNAs-1+RNAp (15+15+15) μg/100 g body weight, to check whether these three RNA preparations are enough effective to normalize the blood glucose level of sick animals when sharing a single administration. The results of the experiment presented in table 24 show that co-administration of all three components in a proper dose (15 μg/100 g body weight) results in even more rapid rate of recovery of pancreatic function than in the most effective experimental group 2 (see table 23).

TABLE 24
Treatment of experimental alloxan type 1 diabetes mellitus in rats with
the combined (RNAbm-1 + RNAs-1 + RNAp) preparation

| | Blood glucose level |
|---|---|
| Time point | (millimoles/l) |
| Background | 18.63 ± 0.18 |
| Day 3 | 10.53 ± 0.28 |
| Day 7 | 9.47 ± 0.32 |
| Day 10 | 7.63 ± 0.26 |
| Day 14 | 6.73 ± 0.18 |
| Day 21 | 5.40 ± 0.15 |
| Day 28 | 5.10 ± 0.17 |
| Day 42 | 5.23 ± 0.15 |

Thus, on the basis of the results of our undertaken series of experiments, it may be considered established that sequential weekly peritoneal injections of the RNAbm-1, RNAs-1, and RNAp preparations of the disclosure at doses of 15 μg/100 g body weight can result in complete functional recovery of the pancreatic islet system in rats with alloxan-induced type 1 diabetes mellitus within three weeks (21 days). Weekly injections of a mixture of equal amounts of these three RNA preparations at summary doses of 15 μg/100 g body weight (5 μg/100 g body weight of each preparation—see experiments 3 and 4 in tables 22 and 23) lead to complete recovery of the pancreatic islet system within 42-45 days. It should be emphasized that the intraperitoneal and intranasal administration routes have proved to be almost equally effective when this protocol of treatment is used. This offers wide possibilities of noninvasive treatment for type 1 diabetes mellitus with the use of the preparations based on natural and at the same time, non-immunogenic components of healthy donor cells proposed in the present application.

Another important result of our experiments on the given model was that the other treatment protocols studied only in terms of the time of recovery were less efficient than the best three ones. Moreover, when the effect of intermediate influence of any of the said six preparations of the disclosure proved not to be optimal, it could be easily corrected by subsequent treatment using one of the optimal protocols. The experimental variants where complete normalization of the blood glucose level took a long time were those where the treatment protocols were not optimal.

The restoration of the functioning of the pancreatic islet system in the given model of type 1 diabetes mellitus had some other important aspects. The restoration processes induced by each one of the three preparations (RNAbm-1, RNAs-1, or RNAp) took a specific period of time. After this period, the glucose content of blood ceased to decrease, but the level reached by that moment was maintained steadily by the regulatory systems of the body. Further restoration of the functioning of the pancreatic islet system induced by another of these RNA preparations also took a specific period of time and led to further decrease in the glucose blood content to a new specific level, below which it did not decrease within 14 days or longer. Only all the three preparations administered in any order ensured a complete restoration of the functioning of the pancreatic insulin-producing system.

Therefore, the three RNA preparations used are qualitatively different from one another and, what is important, are not mutually interchangeable. Apparently, each of them has its specific target.

Finally, it would be pertinent to present evidence that the regulatory preparations described in this application are nontoxic. When we carried out the first preliminary experiments on mice with the genetic model of type 2 diabetes mellitus (Example 9), the optimal doses of the preparations had not been determined yet, so we used single intraperitoneal injections of doses that a priori exceeded the optimal ones (45 to 100 μg/100 g body weight). Further experiments on rats showed that doses from 5 to 15 μg/100 g body weight were sufficient, and these doses were used in the experimental models described above. However, the six- to sevenfold higher doses injected to mice did not cause any noticeable harmful effects; therefore, it can be asserted that the preparations of the disclosure are nontoxic within this wide range of doses.

### Examples 11 and 12

### Treatment of Rats with Experimental Alloxan-Induced Type 1 Diabetes Mellitus (Model (VI))

### Method 2 and Method 3.

In search of further ways of treatment that are not associated with the introduction of xenogeneic for human preparations, which would allow as soon as possible to implement methods of the present invention in medical practice, there have been two additional attempts at treatment of alloxan diabetes on redundant control animals in which 70 days before that date type 1 diabetes was induced by alloxan. To this end, these animals were single i.p. injected of either total RNA preparation isolated from stromal cells of human umbilical cord (mesenchymal stem cells RNA, or RNAmsc) (at doses of 10.0 μg/100 g body weight) in conjunction with the regulatory total RNA preparation isolated from healthy human peripheral blood lymphocytes (hRNApbl) (at doses of 23.4 μg/100 g body weight), or only the RNAmsc preparation from stromal cells of human umbilical cord (at doses of 10.7 μg/100 g body weight).

The results obtained are presented in table 25.

TABLE 25
Treatment of alloxan-induced type 1 diabetes mellitus
with the preparations of total hRNApbl and/or RNAmsc
from stromal cells of human umbilical cord

| | 70 days after alloxan | 70 days after alloxan |
|---|---|---|
| | administration | administration |
| Initial level of | 17.9 (millimoles/l) | 18.3 (millimoles/l) |
| blood glucose | | |
| Administration of | RNAmsc (10.7 μg/100 g) | RNAmsc (10.0 g/100 g) + |
| the preparations | | rRNApbl (23.4 g/100 g) |
| Day 3 | 16.1 (millimoles/l) | 15.5 (millimoles/l) |
| Day 7 | 13.3 (millimoles/l) | 11.0 (millimoles/l) |
| Day 10 | 9.3 (millimoles/l) | 7.8 (millimoles/l) |
| Day 14 | 7.7 (millimoles/l) | 6.1 (millimoles/l) |
| Day 17 | 6.9 (millimoles/l) | 5.8 (millimoles/l) |
| Day 21 | 5.5 (millimoles/l) | 5.6 (millimoles/l) |
| Day 27 | 5.7 (millimoles/l) | 5.6 (millimoles/l) |
| Day 35 | 5.1 (millimoles/l) | 5.3 (millimoles/l) |

Thus, as can be seen from the table, both treatment options are effective, in one of which a preparation of total RNA from the stromal stem cells of human umbilical cord (RNAmsc) is used, and in the other—RNAmsc preparation plus total RNA preparation isolated from health human peripheral blood lymphocytes (hRNApbl).

The given example is, among other things, also an indication that the preparation of total RNA derived from stem cells may be an effective substitute themselves stem cells for treating diseases treatable with stem cells. To those currently refer to diseases selected from the group including, but not limited to amyotrophic lateral sclerosis (ALS), cerebral palsy (CP), epilepsy, spinal cord injury, brain injury and traumatic brain infection, stroke, disease Parkinson's, multiple system atrophy, multiple sclerosis, systemic lupus erythematosus, Devic disease, autoimmune diseases, macular degeneration, retinitis pigmentosa, glaucoma and other eye diseases and visual impairment, diabetes mellitus, diabetic foot, muscular dystrophy, autism and profound developmental delay, progressive supranuclear palsy, corticobasal degeneration, Alzheimer's disease, Huntington's disease, Batten disease, hereditary ataxia, spinocerebellar ataxia, Friedreich's ataxia, cardiomyopathy, congestive heart failure, myocardial infarction (complications), alopecia, arthritis, chronic renal failure, cirrhosis of the liver, lower limb ischemia, osteoporosis and femoral head necrosis, retinopathy of prematurity, neuro-sensory hearing loss, congenital amaurosis Leber.

Despite the high efficiency of total RNA preparation isolated from stem cells from healthy donors, we believe that the combined administration of RNA preparations isolated from stem cells and regulatory RNA preparations isolated, in particular, from peripheral blood lymphocytes of healthy persons, not only increase the effectiveness (see table 25), but also the reliability and safety of the treatment compared to treatment with stem cells as such.

### Example 13

### Effect of the RNAt-3 Preparation Derived from Thymic Lymphoid Cells on Hair Growth

Cosmetic procedures, including hair care, are among the potential applications of the preparations according to the invention. Therefore, we explored the possibility of using the RNAt-1 and RNAt-3 preparations derived from the thymic lymphoid cells of intact rats and rats that had undergone acute blood loss four days earlier, respectively, for regulating (stimulating or inhibiting) hair growth.

For this purpose, we removed hair from symmetric skin areas on both sides of the dorsal spine on the back of outbred black rats. The hairless area was, on average, 4 cm². Then, we daily applied 50 μl of a solution containing 10 μg of either RNAt-1 or RNAt-3 onto the left hairless area of each rat and the same volume of distilled water onto the right (control) one.

On day 17 of this treatment, the new hair growing in the area treated with the RNAt-1 preparation was 6 mm in length, whereas the hair length in the control area of the same rat was 3-4 mm.

On the same day 17 of treatment, the hair growing on the back of the other rat was 3 mm in length in the area treated with the RNAt-3 preparation and 6-8 mm in length in the control area.

Note that the rats used in this experiment considerably differed from each other in hair thickness and length. A rat with thick, long hair was selected for the experiment with hair growth inhibition, because we considered that it would be more difficult to inhibit hair growth in this case. Conversely, a rat with considerably thinner and shorter hair was used in the experiment with hair growth stimulation.

Thus, we demonstrated that the preparations of the invention could regulate growth (in the given case, hair growth) even under the conditions of local external application, which confirms our basic concept. This is an expression of the regulatory effect of RNA derived from cells of the general system regulating cell proliferation. We believe that, in each particular case, an organ-specific component should be added to enhance the effect on a specific target tissue.

Returning to the issue of the administration routes for the preparations of the invention (Example 8), it is now safe to assert that subcutaneous and intramuscular injections are less effective than intravenous, intraperitoneal, and intranasal administrations only as far as the effect at the whole-body level is concerned. The former two routes may ensure exceptionally strong local effects.

Following are some exemplary embodiments of the present invention:

1. A composition comprising a total RNA preparation extracted from an intact lymphoid cell or bone marrow tissue of a healthy donor, and/or from a healthy donor lymphoid cell or bone marrow tissue induced to activate a T-cell population.
2. The composition according to the embodiment 1, wherein the composition or the total RNA preparation modulates proliferation and/or differentiation of a homologous tissue or cell and/or a somatic cell of another histotype.
3. The composition according to the embodiment 1 or 2, wherein the composition or the total RNA preparation isolated from an intact lymphoid cell or bone marrow tissue of a healthy donor, and/or from a healthy donor lymphoid cell or bone marrow tissue induced to activate a T-cell population, is a regulatory.
4. The composition according to the embodiment 1, wherein the activation of a T-cell population occurs ex vivo, in vivo or in vitro.
5. The composition according to the embodiment 2, wherein the modulation of a homologous tissue or cell and/or a somatic cell of another histotype occurs ex vivo, in vivo or in vitro.
6. The composition according to the embodiment 2 or 5, wherein said modulation of proliferation and/or differentiation occurs in a host when the composition or the total RNA preparation is administered.
7. The composition according to the embodiment 1, 2, or 6, wherein the modulation of proliferation and/or differentiation is a stimulation of proliferation or differentiation of a homologous tissue or cell and/or a somatic cell of another histotype.
8. The composition according to the embodiment 7, wherein the total RNA preparation is extracted in a phase when donor cells manifested ability to stimulate proliferation or differentiation of a homologous tissue or cell and/or a somatic cell of another histotype.
9. The composition according to the embodiment 7, wherein said ability to stimulate proliferation or differentiation of a homologous tissue or cell and/or a somatic cell of another histotype occurs from about 15 minutes to about 48 hours after activation of the T-cell population.
10. The composition according to the embodiment 1, 3, or 6, wherein the modulation of proliferation and/or differentiation is an inhibition of proliferation and/or differentiation of a homologous tissue or cell and/or a somatic cell of another histotype.
11. The composition according to the embodiment 10, wherein the total RNA preparation is extracted in a phase when donor cells manifested ability to inhibit proliferation and/or differentiation of a homologous tissue or cell and/or a somatic cell of another histotype.
12. The composition according to the embodiment 10 or 11, wherein said ability to inhibit proliferation or differentiation of a homologous tissue or cell and/or a somatic cell of another histotype occurs from about 48 hours to about 96 hours after activation of the T-cell population.
13. The composition according to any one of the foregoing embodiments, further comprising a total RNA preparation extracted from a healthy donor somatic cell.
14. The composition according to the embodiment 13, wherein said somatic cell is a stem cell.
15. The composition according to any one of the preceding embodiments, wherein the lymphoid cell is a lymphoid cell isolated from a spleen, a thymus, a lymph node, or a population of peripheral blood lymphocytes.
16. The composition according to any one of the preceding embodiments, wherein the donor is a mammal.
17. The composition according to the embodiment 16, wherein the mammalian donor is an allogeneic donor.
18. The composition according to the embodiment 16, wherein the mammalian donor is a human.
19. The composition according to the embodiment 15, wherein the mammalian donor is a xenogeneic donor.
20. The composition according to the embodiment 1, wherein the donor is not a mammal.
21. The composition according to any one of the preceding embodiments, wherein the composition is administered to a mammalian recipient.
22. The composition according to the embodiment 21, wherein the mammalian recipient is a human.
23. The composition according to the embodiment 1-16, 18, 20, wherein the recipient is not a mammal.
24. The composition according to the embodiment 3, wherein the regulatory RNA preparation is extracted from an intact lymphoid cell or an intact bone marrow tissue.
25. The composition according to any one of the embodiments 1 to 24, wherein the healthy donor is young.
26. A composition according to the embodiments 1 to 12, 15 to 25, wherein a regulatory RNA fraction is obtained from the donor.
27. A composition according to the embodiment 26, wherein the regulatory RNA fraction has an average length from about 50 to about 50,000 nucleotides.
28. The composition according to the embodiment 1 comprising the total RNA preparation and a pharmaceutically acceptable carrier, diluent and/or excipient.
29. The composition according to the embodiment 28, presented in a liquid, a lyophilized, or a solid form.
30. The composition according to the embodiment 1 or 28, wherein the composition is formulated for systemic or local administration.
31. The composition according to the embodiment 1, 28 or 30, wherein the administration is intranasal, parenteral, intra-lesional, or topical administration.
32. A method of producing the total RNA preparation according to any one of the embodiments 1 to 27, wherein

from bone marrow tissue or from lymphoid cells of the donor, which are induced to activate a T-cell population
a) in a phase when donor cells manifested ability to stimulate proliferation or differentiation of a homologous tissue or cell and/or a somatic cell of another histotype, or
b) in a phase when donor cells manifested ability to inhibit proliferation and/or differentiation of a homologous tissue or cell and/or a somatic cell of another histotype,
   or

from intact bone marrow tissue or lymphoid cells of the donor,

a total RNA preparation is extracted.

33. The method according to the embodiment 32, wherein the activation occurs ex vivo, in vivo, or in vitro.
34. The method according to the embodiment 32, wherein the activation step occurs in vivo.
35. The method according to the embodiment 32, wherein the activation step occurs in vitro.
36. The method according to the embodiment 32, wherein the activation step occurs ex vivo.
37. The method according to the embodiment 32, wherein the ability to stimulate occurs from about 15 minutes to about 48 hours after activation of the T-cell population.
38. The method according to the embodiment 32, wherein the ability to inhibit occurs from about 48 hours to about 96 hours after activation of the T-cell population.
39. The method according to any one of the embodiments 32-38, wherein the lymphoid cell is a lymphoid cell of a spleen, a thymus, a lymph node, or a population of peripheral blood lymphocytes.
40. The method according to any one of the embodiments 32-38, wherein the donor is a mammal.
41. The method according to the embodiment 40, wherein the mammal is a young and healthy mammal.
42. A method for modulating proliferation and/or differentiation of a somatic target cell in a recipient, comprising administering to the recipient a therapeutically-effective amount of the composition of claim 1, 39, or 66 or the total RNA preparation of claim 1 or 66.
43. The method according to the embodiment 42, wherein the target cell has impaired proliferation and/or differentiation activity.
44. The method according to the embodiment 42 or 43, wherein the target cell is a somatic cell of any histotype.
45. The method according to any one of the embodiments 42 to 44, wherein the recipient is a mammal.
46. The method according to the embodiment 45, wherein the mammal is a human.
47. The method according to any one of the embodiments 43 to 44, wherein the recipient is not a mammal.
48. A method of treating a disease or disorder associated with impaired proliferation and/or differentiation of a somatic target cell(s) of a particular histotype(s), the method comprising administering to a patient a therapeutically-effective amount of the composition of claim 1, 28, or 66, or the total RNA preparation of claim 1 or 66.
49. The method according to the embodiment 48, wherein the disease or disorder associated with impaired proliferation and/or differentiation of a somatic target cell is a degenerative disease or disorder, a neurodegenerative disease or disorder; an autoimmune disease or disorder, hypoproliferative disease or disorder, a hyper-proliferative disease or disorder, a benign neoplastic disorder, a malignant neoplastic disorder; a hereditary defect, a congenital defect, a form of diabetes mellitus, or a disorder treatable with stem cell-based therapy.
50. The method according to the embodiment 49, wherein the neoplastic disease or disorder is prostate adenoma.
51. A method of treating and preventing hematological disease or disorder requiring a blood transfusion or transfusion of blood formed elements, comprising administrating to a patient a therapeutically-effective amount of the composition of claim 1, 28, or 66, or the total RNA preparation of claim 1 or 66 as a complete or partial replacement of blood transfusion.
52. The method according to the embodiment 51, wherein the hematological disease or disorder is anemia.
53. The method according to the embodiment 51, wherein the patient has been exposed to irradiation.
54. The method according to the embodiment 53, wherein the irradiation is a therapy for a tumor disorder.
55. The method according to the embodiment 53 or 54, wherein the hematological disease or disorder results from the irradiation.
56. The method according to the embodiment 51, wherein the patient has been exposed to chemotherapy.
57. The method according to the embodiment 56, wherein the hematological disease or disorder results from the chemotherapy.
58. The method according to any one of the embodiments 53 to 57, wherein the administration to a patient a therapeutically-effective amount of the composition of claim 1 or 28, 66, or the total RNA preparation of claim 1 or 66 is performed from about 15 minutes to about 3 hours before the irradiation or chemotherapy exposure.
59. The method according to the embodiment 49, wherein disorder treatable with stem cell-based therapy, is amyotrophic lateral sclerosis (ALS), cerebral palsy (CP), epilepsy, a spinal cord injury, a brain injury, a traumatic brain infection, a stroke, Parkinson's disease, a multiple system atrophy, multiple sclerosis, systemic lupus erythematosus, Devic disease, an autoimmune disease, macular degeneration, retinitis pigmentosa, glaucoma, eye disease, visual impairment, diabetes mellitus, muscular dystrophy, autism, developmental delay, progressive supranuclear palsy, corticobasal degeneration, Alzheimer's disease, Huntington's disease, Batten's disease, a hereditary ataxia, a spinocerebellar ataxia, a Friedreich's ataxia, cardiomyopathy, chronic heart failure, myocardial infarction, alopecia, arthritis, chronic renal failure, liver cirrhosis, an ischemia of a lower limb, osteoporosis, necrosis of the femoral head, retinopathy of prematurity, a neuro-sensory hearing loss, or congenital amaurosis of Leber.
60. The method according to the embodiment 59, wherein disorder treatable with stem cell-based therapy is a form of diabetes mellitus.
61. The method according to any one of the embodiments 42, 48, or 51, comprising a simultaneous or a sequential administration of a therapeutically-effective amount of a total hRNApbl-1 preparation and/or a total RNA preparation extracted from an umbilical blood and/or a cell or tissue of an umbilical cord.
62. The method according to the embodiment 61, wherein the umbilical cord is a human umbilical cord.
63. A method of treating and preventing a disease or disorder requiring a bone marrow transplantation, comprising administrating to a patient a therapeutically-effective amount of the composition of claim 1, 28, or 66, or the total RNA preparation of claim 1 or 66 as a complete or partial replacement of bone marrow transplantation.
64. The method according to the embodiment 63, wherein the total RNA preparation is extracted from a bone marrow tissue of a healthy donor.
65. A method for improving or reversing a sign(s) or a symptom(s) of aging comprising administering to a subject an effective amount of the composition of claim 1, 39, or 66, or the total RNA preparation of claim 1 or 66.
66. A composition comprising a total RNA preparation produced by the method of claim 32.

1. (canceled)

2. The method of claim 16, wherein the modulation of proliferation and/or differentiation is a stimulation of proliferation and/or differentiation.

3. The method of claim 2, wherein the regulatory total RNA preparation is extracted in a phase when donor cells manifested ability to stimulate proliferation or differentiation of a homologous tissue or cell and/or a somatic cell of another histotype, wherein said ability occurs from about 15 minutes to about 48 hours after activation of the T-cell population.

4. The method of claim 16, wherein the modulation of proliferation and/or differentiation is an inhibition of proliferation and/or differentiation.

5. The method of claim 16, wherein the regulatory total RNA preparation is extracted in a phase when donor cells manifested ability to inhibit proliferation and/or differentiation of a homologous tissue or cell and/or a somatic cell of another histotype, wherein said ability occurs from about 48 hours to about 96 hours after activation of the T cell population.

6. The method of claim 16, wherein the composition further comprises a total RNA preparation extracted from one or more other type(s) of somatic cells of a healthy mammalian donor.

7. The method of claim 6, wherein said somatic cell is a stem cell.

8. The method of claim 16, wherein the lymphoid cell is a lymphoid cell isolated from a spleen, a thymus, a lymph node, and/or a population of peripheral blood lymphocytes of an allogeneic donor and/or a xenogeneic donor.

9. The method of claim 16, wherein the regulatory RNA preparation is extracted from an intact lymphoid cell or an intact bone marrow tissue of a healthy young mammalian donor.

10. The method of claim 16, wherein the composition further comprises a pharmaceutically acceptable carrier, diluent, and/or excipient.

11. The method of claim 16, wherein the composition is a liquid form, a lyophilized form, or a solid form, and wherein the administration is intranasal administration, parenteral administration, intra-lesional administration, or topical administration.

12. The method of claim 16, wherein the total RNA preparation is produced by a method comprising:
extracting a total RNA preparation from intact bone marrow tissue, or lymphoid cells, or somatic cells of any histotype of the healthy donor; or,
extracting a total RNA preparation from intact bone marrow tissue or lymphoid cells of the healthy donor, which are induced to activate a T cell population
in a phase when donor cells manifested ability to stimulate proliferation or differentiation of a homologous tissue or cell and/or a somatic cell of another histotype, or,
in a phase when donor cells manifested ability to inhibit proliferation and/or differentiation of a homologous tissue or cell and/or a somatic cell of another histotype,
wherein the activation occurs ex vivo, in vivo, or in vitro.

13.-14. (canceled)

15. The method of claim 12, wherein the lymphoid cell is a lymphoid cell of a spleen, a thymus, a lymph node, or a population of peripheral blood lymphocytes.

16. A method for modulating proliferation and/or differentiation of a somatic target cell in a recipient, comprising administering to the recipient a therapeutically-effective amount of a composition comprising a purified regulatory total RNA preparation extracted from an intact lymphoid cell or bone marrow tissue of a healthy donor, and/or from a healthy donor lymphoid cell or bone marrow tissue induced to activate a T-cell population.

17. The method of claim 16, wherein the target cell has impaired proliferation and/or differentiation activity.

18. The method of claim 16, wherein the target cell is a somatic cell of any histotype.

19. The method of claim 16, wherein the recipient is a mammal.

20. The method of claim 19, wherein the recipient is a human.

21. A method of treating a disease or disorder associated with impaired proliferation and/or differentiation of a somatic target cell(s) of a particular histotype(s), the method comprising administering to a patient a therapeutically-effective amount of a composition comprising a purified regulatory total RNA preparation extracted from an intact lymphoid cell or bone marrow tissue of a healthy donor, and/or from a healthy donor lymphoid cell or bone marrow tissue induced to activate a T-cell population.

22. The method of claim 21, wherein the disease or disorder associated with impaired proliferation and/or differentiation of a somatic target cell is a degenerative disease or disorder, a neurodegenerative disease or disorder; an autoimmune disease or disorder, hypoproliferative disease or disorder, a hyper-proliferative disease or disorder, a benign neoplastic disorder, a malignant neoplastic disorder; a hereditary defect, a congenital defect, a form of diabetes mellitus, or a disorder treatable with stem cell-based therapy.

23. The method of claim 22, wherein the neoplastic disease or disorder is prostate adenoma.

24. A method of treating and/or preventing hematological disease or disorder requiring a blood transfusion or transfusion of blood formed elements, comprising administrating to a patient a therapeutically-effective amount of a composition comprising a purified regulatory total RNA preparation extracted from an intact lymphoid cell or bone marrow tissue of a healthy donor, and/or from a healthy donor lymphoid cell or bone marrow tissue induced to activate a T-cell population, as a complete or partial replacement of blood transfusion.

25. The method of claim 24, wherein the hematological disease or disorder is anemia.

26. The method of claim 25, wherein the patient has been exposed to irradiation.

27. The method of claim 22, wherein disorder treatable with stem cell-based therapy is amyotrophic lateral sclerosis (ALS), cerebral palsy (CP), epilepsy, a spinal cord injury, a brain injury, a traumatic brain infection, a stroke, Parkinson's disease, a multiple system atrophy, multiple sclerosis, systemic lupus erythematosus, Devic disease, an autoimmune disease, macular degeneration, retinitis pigmentosa, glaucoma, eye disease, visual impairment, diabetes mellitus, muscular dystrophy, autism, developmental delay, progressive supranuclear palsy, corticobasal degeneration, Alzheimer's disease, Huntington's disease, Batten's disease, a hereditary ataxia, a spinocerebellar ataxia, a Friedreich's ataxia, cardiomyopathy, chronic heart failure, myocardial infarction, alopecia, arthritis, chronic renal failure, liver cirrhosis, an ischemia of a lower limb, osteoporosis, necrosis of the femoral head, retinopathy of prematurity, a neuro-sensory hearing loss, or congenital amaurosis of Leber.

28. A method of treating and/or preventing a disease or disorder requiring a bone marrow transplantation, comprising administrating to a patient a therapeutically-effective amount of a composition comprising a purified regulatory total RNA preparation extracted from an intact lymphoid cell or bone marrow tissue of a healthy donor, and/or from a healthy donor lymphoid cell or bone marrow tissue induced to activate a T-cell population, as a complete or partial replacement of bone marrow transplantation.

29. The method of claim 28, wherein the total RNA preparation is extracted from a bone marrow tissue of a healthy donor.

30. (canceled)

31. The method of claim 21, wherein the composition further comprises a total RNA preparation extracted from one or more other type(s) of somatic cells of a healthy mammalian donor.

32. The method of claim 31, wherein said somatic cell is a stem cell.

33. The method of claim 24, wherein the composition further comprises a total RNA preparation extracted from one or more other type(s) of somatic cells of a healthy mammalian donor.

34. The method of claim 28, wherein the composition further comprises a total RNA preparation extracted from one or more other type(s) of somatic cells of a healthy mammalian donor.
